SEQUENCE LISTING (37 sequences)

SEQ ID NO: 1 - protein, 6 aa, artificial sequence
  synthetic peptide
HHHHHH

SEQ ID NO: 2 - protein, 40 aa, artificial sequence
  synthetic peptide
MSPIDPMGHHHHHHGRRRASVAAGILVPRGSPGLDGICSR

SEQ ID NO: 3 - DNA, 891 bp, artificial sequence
  synthetic polynucleotide
atgctcaggggtccgggacccgggcggctgctgctgctagcagtcctgtgcctggggacatcggtgcgctgcaccgaaaccgggaagagcaagaggcagcagagtgtgtgtcgtgcaaggcccatctggtggggaacacagcgccggggctcggagaccatggcgggcgctgcggtgaagtacttaagtcaggaggaggctcaggccgtggaccaagagctttttaacgagtatcagttcagcgtggatcaactcatggagctggccgggttgagctgtgccacggctattgccaaggcttatccccccacgtctatgtccaagagtcccccgactgtcttggtcatctgtggccccggaaataacggaggggatgggctggtctgtgcgcgacacctcaaactttttggttaccagccaactatctattaccccaaaagacctaacaagcccctcttcactgggctagtgactcagtgtcagaaaatggacattcctttccttggtgaaatgcccccagagcccatgatggtggacgagctgtatgagctggtggtggacgccatcttcggcttcagtttcaagggtgacgttcgggagccattccacagcatcctgagtgtcttgagtggactcactgtgcccattgctagcatcgacattccctcaggatgggatgtagagaagggaaaccctagcggaatccaaccagacttactcatctcactgacggcacccaagaagtctgcaactcactttactggccgatatcattaccttgggggtcgctttgtaccacctgctctagagaagaagtaccagctgaacctgccatcttaccctgacacagagtgtgtctaccgtctacagcatcatcatcatcatcattaa

SEQ ID NO: 4 - protein, 296 aa, artificial sequence
  synthetic polypeptide
MLRGPGPGRLLLLAVLCLGTSVRCTETGKSKRQQSVCRARPIWWGTQRRGSETMAGAAVKYLSQEEAQAVDQELFNEYQFSVDQLMELAGLSCATAIAKAYPPTSMSKSPPTVLVICGPGNNGGDGLVCARHLKLFGYQPTIYYPKRPNKPLFTGLVTQCQKMDIPFLGEMPPEPMMVDELYELVVDAIFGFSFKGDVREPFHSILSVLSGLTVPIASIDIPSGWDVEKGNPSGIQPDLLISLTAPKKSATHFTGRYHYLGGRFVPPALEKKYQLNLPSYPDTECVYRLQHHHHHH

SEQ ID NO: 5 - DNA, 1121 bp, homo sapiens
gggccgggccgggccgggggcgcgcgctctgcgagctggatgtccaggctgcgggcgctgctgggcctcgggctgctggttgcgggctcgcgcgtgccgcggatcaaaagccagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcagtgagggaaggtgggtgggtattcttcccaataaagacttagagcccctctcttccagaactgtggattcctgggagctcctctggcaataaaagtcagtgaatggtggaagtcagagaccaaccctggggattgggtgccatctctctaggggtaacacaaagggcaagaggttgctatggtatttggaaacaatgaaaatggactgttagatgccaa

SEQ ID NO: 6 - protein, 288 aa, homo sapiens
MSRLRALLGLGLLVAGSRVPRIKSQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 7 - protein, 40 aa, artificial sequence
  synthetic peptide
MSPIDPMGHHHHHHGRRRASVAAGILVPAASPGLDGICSR

SEQ ID NO: 8 - protein, 304 aa, artificial sequence
  synthetic polypeptide
MSPIDPMGHHHHHHGRRRASVAAGILVPAASPGLDGICSRQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 9 - protein, 11 aa, artificial sequence
  synthetic peptide
GSPGLDGICSR

SEQ ID NO: 10 - protein, 29 aa, artificial sequence
  synthetic peptide
MDYKDHKGKYKDHDIDYKDDDDKLAAANS

SEQ ID NO: 11 - protein, 11 aa, artificial sequence
  synthetic peptide
GSDGDDGDDDR

SEQ ID NO: 12 - DNA, 912 bp, artificial sequence
  synthetic oligonucleotide
atgtcccctatagatccgatgggacatcatcatcatcatcacggaaggagaagggccagtgttgcggcgggaattttggtccctgctgcaagcccaggactcgatggcatatgctcgaggcagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcag

SEQ ID NO: 13 - protein, 6 aa, artificial sequence
  synthetic peptide
LVPRGS

SEQ ID NO: 14 - protein, 29 aa, artificial sequence
  synthetic peptide
MDYKDHKGKYKDHDIDYKDDDDKLAAANS

SEQ ID NO: 15 - protein, 304 aa, artificial sequence
  synthetic polypeptide
MSPIDPMGHHHHHHGRRRASVAAGILVPRGSDGDDGICSRQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 16 - DNA, 912 bp, artificial sequence
  synthetic polynucleotide
atgtcccctatagatccgatgggacatcatcatcatcatcacggaaggagaagggccagtgttgcggcgggaattttggtccctcgtggaagcgatggagacgatggcatatgctcgaggcagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcag

SEQ ID NO: 17 - protein, 27 aa, artificial sequence
  synthetic peptide
MGRRRASVAAGILVPRGSPGLDGICSR

SEQ ID NO: 18 - protein, 19 aa, artificial sequence
  synthetic peptide
MAGILVPRGSPGLDGICSR

SEQ ID NO: 19 - protein, 40 aa, artificial sequence
  synthetic peptide
MSPIDPMGHHHHHHGRRRASVAAGILVPRGSDGDDGDDDR

SEQ ID NO: 20 - DNA, 792 bp, artificial sequence
  synthetic polynucleotide
cagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcag

SEQ ID NO: 21 - protein, 264 aa, artificial sequence
  synthetic polypeptide
QTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 22 - DNA, 888 bp, artificial sequence
  synthetic polynucleotide
atgctcaggggtccgggacccgggcggctgctgctgctagcagtcctgtgcctggggacatcggtgcgctgcaccgaaaccgggaagagcaagaggcagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcag

SEQ ID NO: 23 - protein, 296 aa, artificial sequence
  synthetic polypeptide
MLRGPGPGRLLLLAVLCLGTSVRCTETGKSKRQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 24 - protein, 32 aa, artificial sequence
  synthetic peptide
MLRGPGPGRLLLLAVLCLGTSVRCTETGKSKR

SEQ ID NO: 25 - protein, 272 aa, artificial sequence
  synthetic polypeptide
TETGKSKRQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 26 - protein, 8 aa, artificial sequence
  synthetic peptide
TETGKSKR

SEQ ID NO: 27 - DNA, 923 bp, artificial sequence
  synthetic polynucleotide
atgctcaggggtccgggacccgggcggctgctgctgctagcagtcctgtgcctggggacatcggtgcgctgcaccgaaaccgggaagagcaagaggcagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcagttggtccctcgtggaagccatcatcatcatcatca

SEQ ID NO: 28 - protein, 307 aa, artificial sequence
  synthetic polypeptide
MLRGPGPGRLLLLAVLCLGTSVRCTETGKSKRQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQLVPRGSHHHHH

SEQ ID NO: 29 - protein, 31 aa, artificial sequence
  synthetic peptide
LRGPGPGRLLLLAVLCLGTSVRCTETGKSKR

SEQ ID NO: 30 - protein, 283 aa, artificial sequence
  synthetic polypeptide
TETGKSKRQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQLVPRGSHHHHH

SEQ ID NO: 31 - DNA, 1198 bp, artificial sequence
  synthetic polynucleotide
atggactacaaagaccatgacggtgattataaagatcatgacatcgattacaaggatgacgatgacaagcttgcggccgcgaattcagggccgggggcgcgcgctctgcgagctggatgtccaggctgcgggcgctgctgggcctcgggctgctggttgcgggctcgcgcgtgccgcggatcaaaagccagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcagtgagggaaggtgggtgggtattcttcccaataaagacttagagcccctctcttccagaactgtggattcctgggagctcctctggcaataaaagtcagtgaatggtggaagtcagagaccaaccctggggattgggtgccatctctctaggggtaacacaaagggcaagaggttgctatggtatttggaaacaatgaaaatggactgttagatgccaa

SEQ ID NO: 32 - protein, 317 aa, artificial sequence
  synthetic polypeptide
MDYKDHDGDYKDHDIDYKDDDDKLAAANSMSRLRALLGLGLLVAGSRVPRIKSQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 33 - protein, 29 aa, artificial sequence
  synthetic peptide
MDYKDHDGDYKDHDIDYKDDDDKLAAANS

SEQ ID NO: 34 - DNA, 879 bp, artificial sequence
  synthetic polynucleotide
atggactacaaagaccatgacggtgattataaagatcatgacatcgattacaaggatgacgatgacaagcttgcggccgcgaattcacagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcag

SEQ ID NO: 35 - protein, 293 aa, artificial sequence
  synthetic polypeptide
MDYKDHDGDYKDHDIDYKDDDDKLAAANSQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 36 - protein, 304 aa, artificial sequence
  synthetic polypeptide
MSPIDPMGHHHHHHGRRRASVAAGILVPRGSDGDDGDDDRQTIACRSGPTWWGPQRLNSGGRWDSEVMASTVVKYLSQEEAQAVDQELFNEYQFSVDQLMELAGLSCATAIAKAYPPTSMSRSPPTVLVICGPGNNGGDGLVCARHLKLFGYEPTIYYPKRPNKPLFTALVTQCQKMDIPFLGEMPAEPMTIDELYELVVDAIFGFSFKGDVREPFHSILSVLKGLTVPIASIDIPSGWDVEKGNAGGIQPDLLISLTAPKKSATQFTGRYHYLGGRFVPPALEKKYQLNLPPYPDTECVYRLQ

SEQ ID NO: 37 - DNA, 912 bp, artificial sequence
  synthetic polynucleotide
atgtcccctatagatccgatgggacatcatcatcatcatcacggaaggagaagggccagtgttgcggcgggaattttggtccctcgtggaagcgatggagacgatggcgatgacgacaggcagaccatcgcctgtcgctcgggacccacctggtggggaccgcagcggctgaactcgggtggccgctgggactcagaggtcatggcgagcacggtggtgaagtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgtatgagctggtggtggatgccatctttggcttcagcttcaagggcgatgttcgggaaccgttccacagcatcctgagtgtcctgaagggactcactgtgcccattgccagcatcgacattccctcaggatgggacgtggagaagggaaatgctggagggatccagccagacttgctcatatccctcacagcccccaaaaaatctgcaacccagtttaccggtcgctaccattacctggggggtcgttttgtgccacctgctctggagaagaagtaccagctgaacctgccaccctaccctgacaccgagtgtgtctatcgtctgcag

(19) United States
(12) Patent Application Publication
(10) Pub. No.: US 2024/0209341 A1
(43) Pub. Date: Jun. 27, 2024

(54) COMPOSITIONS AND METHODS FOR TARGETING INFLAMMATORY OR ACTIVATED CELLS AND TREATING OR AMELIORATING INFLAMMATORY CONDITIONS AND PAIN

(71) Applicant: The Regents of the University of California, Oakland, CA (US)

(72) Inventors: Yury MILLER, San Diego, CA (US); Soo-Ho CHOI, San Diego, CA (US)

(21) Appl. No.: 18/282,749

(22) PCT Filed: Mar. 18, 2022
(86) PCT No.: PCT/US2022/020991

§ 371 Date: Sep. 18, 2023

Related U.S. Application Data
(60) Provisional application No. 63/162,714, filed on Mar. 18, 2021.

(51) Int. Cl. C12N 9/90; A61K 38/52; A61P 11/06; A61P 25/00; A61P 27/06

(52) U.S. Cl. CPC C12N 9/90; A61K 38/52; A61P 11/06; A61P 25/00; A61P 27/06; C12Y 501/99006; C07K 2319/02; C07K 2319/50

(57) ABSTRACT

Provided are methods for modification of amino acid sequence and increasing levels of expression of ApoA-I Binding Protein to treat: a neuropathic pain, a CNS inflammation, an allodynia, a post nerve injury pain, a post-surgical pain, a chemotherapeutic-induced peripheral neuropathy, a neurodegeneration, including for example, a neurodegenerative disease or condition such as Alzheimer's disease, a hyperalgesia, primary headaches such as migraines and cluster headaches, glaucoma or other inflammatory diseases of the eye, lung inflammation, asthma, HIV infection, vascular inflammation, atherosclerosis and cardiovascular disease. Provided are methods comprising administering pharmaceutical compositions comprising a recombinantly modified APOA1BP polypeptide to treat a neuropathic pain, an allodynia, a hyperalgesia, a neurodegenerative disease, a primary headache such as a migraine, glaucoma, lung inflammation and asthma, acute respiratory distress syndrome (ARDS), sepsis, viral infection, including influenza, coronavirus (for example, COVID-19) or HIV infection, or its comorbidities, and/or vascular inflammation, atherosclerosis and cardiovascular disease.

### RELATED APPLICATIONS

This Patent Convention Treaty (PCT) International Application claims the benefit of priority under 35 U.S.C. § 119(e) of U.S. Provisional Application Ser. No. (USSN) 63/162,714, filed Mar. 18, 2021. The aforementioned application is expressly incorporated herein by reference in its entirety and for all purposes. All publications, patents, patent applications cited herein are hereby expressly incorporated by reference for all purposes.

### GOVERNMENT RIGHTS

This invention was made with government support under grants NS102432, NS104769, HL135737, AI147879 and HL136275, awarded by the National Institutes of Health (NIH). The government has certain rights in the invention.

### TECHNICAL FIELD

This invention generally relates to medicine, inflammation, pain control and cell biology. In particular, in alternative embodiments, provided are methods for modification of structure and increasing levels of expression of ApoA-I Binding Protein (APOA1BP, AIBP, or AI-BP, also known as NAD(P)HX Epimerase or NAXE) to treat, ameliorate, prevent, reverse, decrease the severity and/or duration of: a neuropathic pain, a CNS inflammation, an allodynia, a post nerve injury pain, a post-surgical pain, a chemotherapeutic-induced peripheral neuropathy (CIPN) (for example, cisplatin-induced allodynia), a neurodegeneration, including for example, a neurodegenerative disease or condition such as Alzheimer's disease, a hyperalgesia, primary headaches such as migraines and cluster headaches, glaucoma, lung inflammation and asthma, HIV infection and its comorbidities, and/or vascular inflammation and cardiovascular disease. In alternative embodiments, provided are methods comprising structural modifications and administering formulations and pharmaceutical compositions comprising an recombinantly modified APOA1BP polypeptide or protein that is a human or a mammalian APOA1BP, or a peptidomimetic or a synthetic APOA1BP, or a bioisostere thereof, to treat, ameliorate prevent, reverse, decrease the severity of a neuropathic pain, an allodynia, a hyperalgesia, a neurodegenerative disease or condition such as Alzheimer's disease, a primary headache such as a migraine, glaucoma or other inflammatory diseases of the eye, lung inflammation and asthma, acute respiratory distress syndrome (ARDS), sepsis, viral infection, including influenza, coronavirus (for example, COVID-19) or HIV infection, or its comorbidities, and/or vascular inflammation, atherosclerosis and cardiovascular disease.

### BACKGROUND

Apolipoprotein A-I Binding Protein, or ApoA-I binding protein (AIBP), also called NAXE, NAD(P)HX epimerase, is a protein discovered in a screen of proteins that physically associate with apoA-I.

Regulation of cholesterol metabolism in the context of neurodegeneration and specifically Alzheimer's disease (AD) received ample attention due in part to strong association between APOE polymorphism and the risk of AD. However, the role of cholesterol regulation as a factor in the development of chronic pain states remains unknown. Chemotherapy-induced peripheral neuropathy (CIPN) is one of the debilitating adverse effects of antineoplastic drug usage during cancer treatment, affecting over 50% of patients undergoing chemotherapy (Seretny et al., 2014). Neuroinflammation mediated by glial cell activation and infiltrating immune cells in the spinal cord and dorsal root ganglia is an important component of CIPN and other neuropathies (Lees et al., 2017; Makker et al., 2017). Glial cells express toll-like receptor-4 (TLR4), which mediates secretion of inflammatory cytokines, chemokines, and bioactive lipids (Bruno et al., 2018; Gregus et al., 2018; Papageorgiou et al., 2016). In addition, CIPN-associated activation of TLR4 signaling has been reported in dorsal root ganglion nociceptors (Chen et al., 2017; Li et al., 2021). Systemic deficiency of TLR4 or its signaling adaptor molecules MyD88 and TRIF, alone or in combination, attenuates and prevents hyperalgesia and allodynia in mice treated with cisplatin (Hu et al., 2018; Pevida et al., 2013; Yan et al., 2019). However, the cell type in which TLR4 activation induces allodynia is unknown.

### SUMMARY

In alternative embodiments, provided are isolated or recombinant polypeptides, or chimeric polypeptide, wherein the polypeptide is comprised of (or comprises) a ApoA-I Binding Protein (AIBP) amino acid sequence and an amino acid sequence N-terminal to the AIBP amino acid sequence,
wherein the amino acid sequence N-terminal to the AIBP amino acid sequence is comprised of at least eight amino acids, or the amino acid sequence N-terminal to the AIBP amino acid sequence is 5, 6, 7, 8, 9, 10, 11, 12 13, 14, 15, or 16 or more amino acids in length,
wherein the amino acid sequence N-terminal to the AIBP amino acid sequence is capable of inducing unfolding, exposing or otherwise making accessible the cryptic domain in the AIBP amino acid sequence for binding of the polypeptide to TLR4 under relevant physiological conditions,
wherein optionally “relevant physiological conditions” refer to those conditions to be experienced by the polypeptide compound in vivo upon providing it to a subject in need thereof by administration,
with the proviso that the amino acid sequence N-terminal to the AIBP amino acid sequence is not comprised of a His-tag and a proteolytic cleavage site that when acted upon under said conditions results in loss of the His-tag.

In alternative embodiments, of isolated or recombinant polypeptides, or chimeric polypeptide, as provided herein:
the amino acid sequence N-terminal to the AIBP amino acid sequence is comprised of between about 8 and about 40 contiguous amino acid residues (or 5, 6, 7, 8, 9, 10, 11, 12, 13, 14, 15, 16, 17 18, 19 or 20 or more contiguous amino acid residues) of which between about 3 and about 12, or between about 8 and 20, or between about 10 and 40, amino acid residues are independently selected from the group consisting of arginine (R), histidine (H) and lysine (K);
the N-terminus of the amino acid sequence N-terminal to the AIBP amino acid sequence is or comprises a secretion signal amino acid sequence, and optionally the secretion signal amino acid sequence is (or comprises) a fibronectin secretion signal domain, an immunoglobulin heavy chain secretion signal domain, an immunoglobulin kappa light chain secretion signal domain, or an interleukin-2 signal peptide secretion signal domain, and optionally the fibronectin secretion signal domain is MLRGPGPGRLLLLAVLCLGTSVRCTETGKSKR (SEQ ID: NO:24):
the AIBP sequence is hAIBP (SEQ ID NO:6, or is encoded by SEQ ID NO:5) or d24hAIBP (SEQ ID NO:21, or encoded by SEQ ID NO:20);
the amino acid sequence N-terminal to the AIBP amino acid sequence is comprised of about 6, or between about 5 and 40, consecutive histidine amino acid residues (for example, HHHHHH (SEQ ID NO:1)), N-terminal to the TLR4 binding domain of the AIBP amino acid sequence;
the polypeptide has (or comprises) a thrombin cleavage domain intervening between the N-terminus of the TLR4 binding domain of the ApoA-I Binding Protein sequence, wherein the thrombin cleavage domain has one or more amino acid deletions and/or mutations within this domain so as to render it functionally inoperable;
the amino acid sequence N-terminal to the AIBP amino acid sequence is: MSPIDPMGHHHHHHGRRRASVAAGILVPRGSPGLDGICSR (SEQ ID NO:2) or MSPIDPMGHHHHHHGRRRASVAAGILVPRGSDGDDGDDDR (SEQ ID NO:19), each having an amino acid mutation of its thrombin cleavage domain so as to render it functionally inoperative;
the amino acid sequence N-terminal to the AIBP amino acid sequence is selected from the group consisting of: TETGKSKR (SEQ ID NO:26); MDYKDHDGDYKDHDIDYKDDDDKLAAANS (SEQ ID NO:33), or MSPIDPMGHHHHHHGRRRASVAAGILVPAASPGLDGICSR (SEQ ID NO:7);
the AIBP amino acid sequence is that of (or is derived from) a mammalian AIBP amino acid sequence, and optionally the mammalian AIBP amino acid sequence is that of (or is derived from) a human AIBP amino acid sequence; and/or
the human AIBP amino acid sequence is (or comprises the full-length amino acid sequence of 288 amino acid residues with NCBI Reference Sequence: NP_658985.2, or optionally the human AIBP amino acid sequence is the human AIBP amino acid sequence with NCBI Reference Sequence: NP_658985.2 having deletion of amino acids 1-24 from said AIBP amino acid sequence.

In alternative embodiments, provided are pharmaceutical compositions or formulations comprised of (or comprising) a polypeptide compound as provided herein and at least one excipient suitable for (of formulated for) parenteral administration. In alternative embodiments, the parenteral administration is by intrathecal injection or intrathecal implant, or by intravenous or intracular injection.

In alternative embodiments, provided are nucleic acids, wherein the nucleic acid compound is comprised of (or comprises) a nucleic acid sequence that encodes for the polypeptide as provided herein.

In alternative embodiments, provided are expression vectors comprised of (or comprising, or having contained therein) a nucleic acid sequence that encodes for a polypeptide as provided herein. The expression vector can be a recombinant virus such as a recombinant adenovirus or a recombinant lentivirus.

In alternative embodiments, provided are methods and uses for treating, ameliorating, preventing, reversing or decreasing the severity or duration of, or decreasing the severity of symptoms of:
neuropathic pain,
inflammation-induced neuropathic pain,
   wherein optionally the inflammation-induced neuropathic pain comprises a Toll-like receptor 4 (TLR4)-mediated inflammation-induced neuropathic pain,
nerve or CNS inflammation,
   wherein optionally the nerve or CNS inflammation comprises a TLR4-mediated nerve or CNS inflammation,
allodynia,
   wherein optionally the allodynia comprises a TLR4-mediated allodynia,
a post nerve or tissue injury pain or neuropathic pain,
   wherein optionally the post nerve or tissue injury pain or neuropathic pain is generated or caused by, or is a sequelae to, trauma, chemotherapy, arthritis, diabetes, or viral infection,
post-surgical pain or neuropathic pain,
chemotherapeutic-induced peripheral neuropathy (CIPN) (for example, a cisplatin-induced CIPN or allodynia),
a neurodegenerative disease or condition, optionally a chronic or progressive neurodegenerative disease or condition, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS),
a primary headache, optionally a migraine or a cluster headache,
hyperalgesia,
glaucoma or other inflammatory diseases of the eye,
lung inflammation and asthma,
acute respiratory distress syndrome (ARDS),
sepsis,
viral infection, and optionally the virus comprises an influenza or a coronavirus (optionally the coronavirus is COVID-19) or a human immunodeficiency virus (HIV) or a virus causing an HIV infection, (optionally an influenza A, B or C), or a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection, or its comorbidities, and/or
vascular inflammation, atherosclerosis and cardiovascular disease,
in a subject by adding or increasing levels of an ApoA-I Binding Protein (APOA1BP, AIBP, or AI-BP),
wherein the method comprises:
(a) providing a formulation or a pharmaceutical composition comprising:
(i) a recombinant or synthetic ApoA-I Binding Protein (APOA1BP, AIBP, or AI-BP) polypeptide compound or composition having a heterologous (or non-native, or non-AIBP, or non-wild type (wt), or any sequence not present in wild type (wt) AIBP) amino terminus amino acid sequence of at least about ten amino acids, or between about 5 to 20 amino acids, or between about 10 to 100 amino acids, or between about 20 to 80 amino acids, or between about 30 to 50 amino acids, or having on the AIBP amino terminus 5, 6, 7, 8, 9, 10, 11, 12, 13, 14, 15, 16, 17, 18, 19, 20, 21, 22, 23, 24, 25, 26, 27, 28, 29 or 30 or more amino acid residues that are not present in wt AIBP or are non-native (to AIBP) amino acid residues or peptides (also called AIBP variants as provided herein),
and optionally with the proviso that the amino acid sequence N-terminal to the AIBP amino acid sequence is not comprised of a His-tag and a proteolytic cleavage site that when acted upon under a physiological condition (for example, in a cellular milieu or equivalent, or intracellularly) results in loss of the His-tag,
and optionally the heterologous (or non-wild type, or non-native) amino terminus amino acid sequence (or amino acid residues) comprises a peptide tag, and optionally the peptide tag comprises a multi-histidine (multi-his) tag, and optionally the multi-his tag comprises six histidines (HHHHHH (SEQ ID NO:1)), or 3, 4, 5, 6, 7, 8, 9, 10, 11, 12, 13, 14, 15, 16, 17, 18, 19, or 20 or more histidine residues,
and optionally the heterologous (or non-wild type) amino terminus amino acid sequence comprises an enzyme cleavage site, and optionally the enzyme cleavage site comprises a thrombin cleavage site,
and optionally the heterologous (or non-wild type) amino terminus amino acid sequence comprises a secretion signal, and optionally the secretion signal comprises a fibronectin secretion signal (for example, SEQ ID NO:24), an immunoglobulin heavy chain secretion signal or an immunoglobulin kappa light chain secretory peptide, or an interleukin-2 signal peptide,
and optionally the heterologous (or non-wild type) amino terminus amino acid sequence comprises the amino acid sequence MSPIDPMGHHHHHHGRRRASVAAGILVPRGSPGLDGICSR (SEQ ID NO:2),
wherein all these AIBP variants as provided herein (or, all the AIBP amino acids that also comprise an amino terminal sequence not present in wt AIPB, or comprise a heterologous amino terminal peptide or amino acid residues) are capable of, or serve the purpose of, in physiologic conditions, cause unfolding or exposing or making accessible a cryptic domain in the AIBP molecule that comprises of amino acids 25-51, which mediates AIBP binding to a toll-like receptor-4 (TLR4) polypeptide (in other words, the AIBP variants as provided herein have a TLR4 binding domain exposed to the extracellular milieu such that the AIBP variants as provided herein can bind to TLR4 polypeptide under physiologic conditions);
(ii) a recombinant nucleic acid encoding the APOA1BP polypeptide of (i),
and optionally the nucleic acid that expresses or encodes a APOA1BP polypeptide or a polypeptide having a APOA1BP polypeptide activity is contained in an expression vehicle, vector, recombinant virus, or equivalent,
and optionally the vector or virus is or comprises an adenovirus vector or an adeno-associated virus (AAV) vector, a retrovirus, a lentiviral vector, a herpes simplex virus, a human immunodeficiency virus (HIV), or a synthetic vector,
and optionally the AAV vector comprises or is:
an adeno-associated virus (AAV), or an adenovirus vector,
an AAV serotype or variant AAV5, AAV6, AAV8 or AAV9, AAV-DJ or AAV-DJ/8™ (Cell Biolabs, Inc., San Diego, CA),
a rhesus-derived AAV, or the rhesus-derived AAV AAVrh. 10hCLN2,
an AAV capsid mutant or AAV hybrid serotype,
an organ-tropic AAV, or a cardiotropic AAV, or a cardiotropic AAVM41 mutant,
wherein optionally the AAV is engineered to increase efficiency in targeting a specific cell type that is non-permissive to a wild type (wt) AAV and/or to improve efficacy in infecting only a cell type of interest,
and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;
(iii) a formulation or pharmaceutical composition comprising a recombinant or synthetic ApoA-I Binding Protein (APOA1BP, AIBP, or AI-BP) polypeptide or protein of (i), or a recombinant nucleic acid of (ii), wherein optionally the recombinant or synthetic ApoA-I Binding Protein (APOA1BP, AIBP, or AI-BP) polypeptide or protein is or comprises all or part of a human or a mammalian APOA1BP, or a AIBP1 or a AIBP2 sequence;
(iv) a formulation or pharmaceutical composition of (iii), formulated for administration in vivo; or formulated for enteral or parenteral administration, or for oral, intravenous (IV) or intrathecal (IT) administration,
wherein optionally the formulation or pharmaceutical composition, or the recombinant, peptidomimetic or a synthetic APOA1BP, or bioisostere of APOA1BP, or nucleic acid encoding the APOA1BP, or vector having contained therein a nucleic acid encoding the APOA1BP, is carried in a nanoparticle, a particle, a micelle or a liposome or lipoplex, a polymersome, a polyplex or a dendrimer, which optionally can further comprise or express a cell or CNS penetrating moiety or peptide or a CNS targeting moiety or peptide; or
(v) a formulation or pharmaceutical composition of any of (iii) to (iv), formulated for as a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant (for example, an intrathecal implant); and
(b) administering: recombinant or synthetic ApoA-I Binding Protein (APOA1BP, AIBP, or AI-BP) polypeptide or protein of (a)(i), or a recombinant nucleic acid of (a)(ii), or the formulation or the pharmaceutical composition of (a)(iii) or (a)(iv) to a subject or individual in need thereof, wherein optionally the subject or individual is a mammal, a human or an animal,
thereby treating, ameliorating, preventing, reversing or decreasing the severity or duration of, or decreasing the severity of symptoms of, the:
neuropathic pain,
inflammation-induced neuropathic pain,
   wherein optionally the inflammation-induced neuropathic pain comprises a Toll-like receptor 4 (TLR4)-mediated inflammation-induced neuropathic pain,
nerve or CNS inflammation,
   wherein optionally the nerve or CNS inflammation comprises a TLR4-mediated nerve or CNS inflammation,
allodynia,
   wherein optionally the allodynia comprises a TLR4-mediated allodynia,
a post nerve or tissue injury pain or neuropathic pain,
   wherein optionally the post nerve or tissue injury pain or neuropathic pain is generated or caused by, or is a sequela to, trauma, chemotherapy, arthritis, diabetes, or viral infection,
post-surgical pain or neuropathic pain,
chemotherapeutic-induced peripheral neuropathy (CIPN) (for example, a cisplatin-induced CIPN or allodynia),
a neurodegenerative disease or condition, optionally a chronic or progressive neurodegenerative disease or condition, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS),
a primary headache, optionally a migraine or a cluster headache,
hyperalgesia,
glaucoma or other inflammatory diseases of the eye,
lung inflammation and asthma,
acute respiratory distress syndrome (ARDS),
sepsis,
viral infection, and optionally the virus comprises an influenza or a coronavirus (optionally the coronavirus is COVID-19) or a human immunodeficiency virus (HIV) or a virus causing an HIV infection, (optionally an influenza A, B or C), or a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection, and/or,
vascular inflammation, atherosclerosis and cardiovascular disease.

In alternative embodiments, provided are kits comprising: a recombinant or synthetic ApoA-I Binding Protein (APOA1BP, AIBP, or AI-BP) polypeptide or protein; a recombinant nucleic acid; and/or a formulation or a pharmaceutical composition as used in a method as provided herein, and optionally comprising instructions on practicing a method as provided herein.

In alternative embodiments, provided are uses of a formulation or a pharmaceutical composition as provided herein, in the manufacture of a medicament.

In alternative embodiments, provided are uses of a formulation or a pharmaceutical composition as provided herein, in the manufacture of a medicament for treating, ameliorating, preventing, reversing or decreasing the severity or duration of, or decreasing the severity of symptoms of:
neuropathic pain,
inflammation-induced neuropathic pain,
   wherein optionally the inflammation-induced neuropathic pain comprises a Toll-like receptor 4 (TLR4)-mediated inflammation-induced neuropathic pain,
nerve or CNS inflammation,
   wherein optionally the nerve or CNS inflammation comprises a TLR4-mediated nerve or CNS inflammation,
allodynia,
   wherein optionally the allodynia comprises a TLR4-mediated allodynia,
a post nerve or tissue injury pain or neuropathic pain, wherein optionally the post nerve or tissue injury pain or neuropathic pain is generated or caused by, or is a sequelae to, trauma, chemotherapy, arthritis, diabetes, or viral infection,
post-surgical pain or neuropathic pain,
chemotherapeutic-induced peripheral neuropathy (CIPN) (for example, a cisplatin-induced CIPN or allodynia),
a neurodegenerative disease or condition, optionally a chronic or progressive neurodegenerative disease or condition, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS),
a primary headache, optionally a migraine or a cluster headache,
hyperalgesia,
glaucoma or other inflammatory diseases of the eye,
lung inflammation and asthma,
acute respiratory distress syndrome (ARDS),
sepsis,
viral infection, and optionally the virus comprises an influenza or a coronavirus (optionally the coronavirus is COVID-19) or a human immunodeficiency virus (HIV) or a virus causing an HIV infection, (optionally an influenza A, B or C), or a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection, or its comorbidities, and/or
vascular inflammation, atherosclerosis and cardiovascular disease.

In alternative embodiments, provided are a formulation, a pharmaceutical composition or a therapeutic combination for use in a method for treating, ameliorating, preventing, reversing or decreasing the severity or duration of, or decreasing the severity of symptoms of:
neuropathic pain,
inflammation-induced neuropathic pain,
   wherein optionally the inflammation-induced neuropathic pain comprises a Toll-like receptor 4 (TLR4)-mediated inflammation-induced neuropathic pain,
nerve or CNS inflammation,
   wherein optionally the nerve or CNS inflammation comprises a TLR4-mediated nerve or CNS inflammation,
allodynia,
   wherein optionally the allodynia comprises a TLR4-mediated allodynia,
a post nerve or tissue injury pain or neuropathic pain,
   wherein optionally the post nerve or tissue injury pain or neuropathic pain is generated or caused by, or is a sequelae to, trauma, chemotherapy, arthritis, diabetes, or viral infection,
post-surgical pain or neuropathic pain,
chemotherapeutic-induced peripheral neuropathy (CIPN) (for example, a cisplatin-induced CIPN or allodynia),
a neurodegenerative disease or condition, optionally a chronic or progressive neurodegenerative disease or condition, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS),
a primary headache, optionally a migraine or a cluster headache,
hyperalgesia,
glaucoma or other inflammatory diseases of the eye,
lung inflammation and asthma,
acute respiratory distress syndrome (ARDS),
sepsis,
viral infection, and optionally the virus comprises an influenza or a coronavirus (optionally the coronavirus is COVID-19) or a human immunodeficiency virus (HIV) or a virus causing an HIV infection, (optionally an influenza A, B or C), or a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection, or its comorbidities, and/or.
vascular inflammation, atherosclerosis and cardiovascular disease,
wherein the formulation or the therapeutic combination comprises a formulation or a therapeutic combination as provided herein,
and wherein the formulation or a therapeutic combination is administered to an individual or patient in need thereof.

In alternative embodiments, provided are methods for exposing the cryptic (or hidden, unexposed, unaccessible) N-terminal TLR4-binding domain of an ApoA-I Binding Protein (APOA1BP, AIBP, or AI-BP) polypeptide, comprising adding to a native (or wild type) AIBP polypeptide a heterologous (or non-native, or non-wild type) amino terminus amino acid sequence of at least about ten amino acid, or between about 5 to 50 amino acids, or between about 10 to 100 amino acids, or between about 20 to 80 amino acids, or between about 30 to 50 amino acids, or adding to the AIBP amino terminus 5, 6, 7, 8, 9, 10, 11, 12, 13, 14, 15, 16, 17, 18, 19, 20, 21, 22, 23, 24, 25, 26, 27, 28, 29 or 30 or more amino acid residues that are not present in wt AIBP or that are non-native (non-AIBP) amino acid residues or peptides,
and optionally the heterologous amino terminus amino acid sequence comprises a peptide tag, and optionally the peptide tag comprises a multi-histidine (multi-his) tag, and optionally the multi-his tag comprises at least six histidines (HHHHHH (SEQ ID NO:1)), or 3, 4, 5, 6, 7, 8, 9, 10, 11, 12, 13, 14, 15, 16, 17, 18, 19, or 20 or more histidine residues,
and optionally the heterologous amino terminus amino acid sequence comprises an enzyme cleavage site, and optionally the enzyme cleavage site comprises a thrombin cleavage site,
and optionally the heterologous amino terminus amino acid sequence comprises a secretion signal, and optionally the secretion signal comprises a fibronectin secretion signal, an immunoglobulin heavy chain secretion signal or an immunoglobulin kappa light chain secretory peptide, or an interleukin-2 signal peptide,
and optionally the heterologous amino terminus amino acid sequence comprises the amino acid sequence
MSPIDPMGHHHHHHGRRRASVAAGILVPRGSPGLDGICSR (SEQ ID NO:2).

In alternative embodiments, provided are polypeptide compounds, wherein a polypeptide compound is comprised of a ApoA-I Binding Protein (AIBP) amino acid sequence and an amino acid sequence N-terminal to the AIBP amino acid sequence, wherein the amino acid sequence N-terminal to the AIBP amino acid sequence is comprised of at least eight amino acids, or between 4 and 12 amino acids, or between 5 and 10 amino acids, wherein the amino acid sequence N-terminal to the AIBP amino acid sequence is capable of inducing unfolding, exposing or otherwise making accessible the cryptic domain in the AIBP amino acid sequence for binding of the polypeptide to TLR4 under relevant physiological conditions, with the proviso that the amino acid sequence N-terminal to the AIBP amino acid sequence is not comprised of a His-tag and a proteolytic cleavage site that when acted upon under said physiological conditions results in loss of the His-tag.

In alternative embodiments, provided are methods for treating, ameliorating, preventing, reversing or decreasing the severity or duration of a TLR4-mediated disease or condition by providing to a subject in need thereof:
a pharmaceutically acceptable composition comprising a polypeptide compound or a nucleic acid compound as provided herein, wherein the nucleic acid sequence of the nucleic acid compound encodes the amino acid sequence of said polypeptide,
wherein the TLR4-mediated diseases or conditions include, but are not limited to, inflammation-induced pain, CNS inflammatory diseases and conditions, arthritis, neurodegenerative diseases and conditions, allodynia, hyperalgesia, lung inflammatory diseases or conditions, ocular inflammatory diseases and conditions, sepsis, vascular inflammatory diseases and conditions, diseases and conditions generated or caused by, or sequela to posttraumatic stress disorder, traumatic war neuroses, post-traumatic stress syndromes (PTSS), and viral infections.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings set forth herein are illustrative of embodiments provided herein and are not meant to limit the scope of the invention as encompassed by the claims.

FIG. 1A-F illustrate data showing that chemotherapy-induced peripheral neuropathy alters TLR4 dimerization and lipid rafts in spinal microglia, and reversal by AIBP:

FIG. 1A graphically illustrates data showing withdrawal thresholds in wild type (WT) mice in response to intraperitoneal (i.p.) cisplatin (2 injections of 2.3 mg/kg/day), followed by a single dose of intrathecal (i.t.) saline (5 μl) or AIBP (0.5 μg/5 μl); naïve mice received no injections;

FIG. 1B-C graphically illustrate data showing an analysis of CD11b⁺/TMEM119⁺ spinal microglia cells showing TLR4 dimerization (FIG. 1B) and lipid raft content measured by CTxB staining (FIG. 1C) 24 hours after i.t. saline or AIBP, i.e. at day 8 of the time course shown in FIG. 1A;

FIG. 1C illustrates images of BV-2 microglia cells (left panels) incubated for 30 min with AIBP (0.2 μg/mL) or vehicle in complete media, followed by a 5 min incubation with LPS (100 ng/mL), and graphically illustrates (right panel) data showing the Manders' coefficients (a colocalization analysis) with or without LPS and/or AIBP; and

FIG. 1E-F graphically illustrate data showing AIBP levels over time in CSF (FIG. 1E) and lumbar spinal cord (FIG. 1F), as discussed in detail in Example 1, below.

FIG. 2A-C illustrate data showing gene expression in spinal microglia of CIPN mice:

FIG. 2A-B illustrate data from studies where microglia (CD11b⁺TEMEM119⁺) were FACS-sorted from 3 groups shown in FIG. 1A,

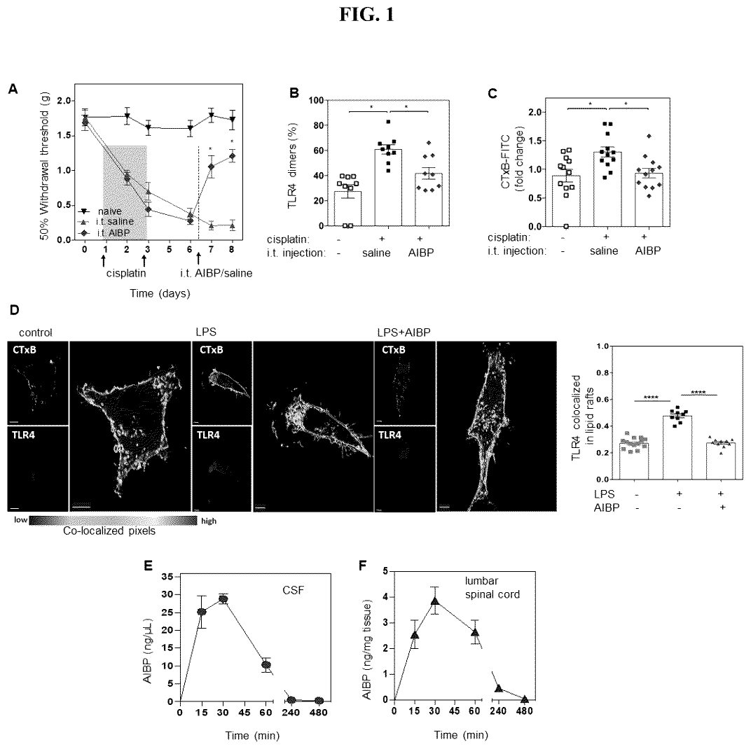

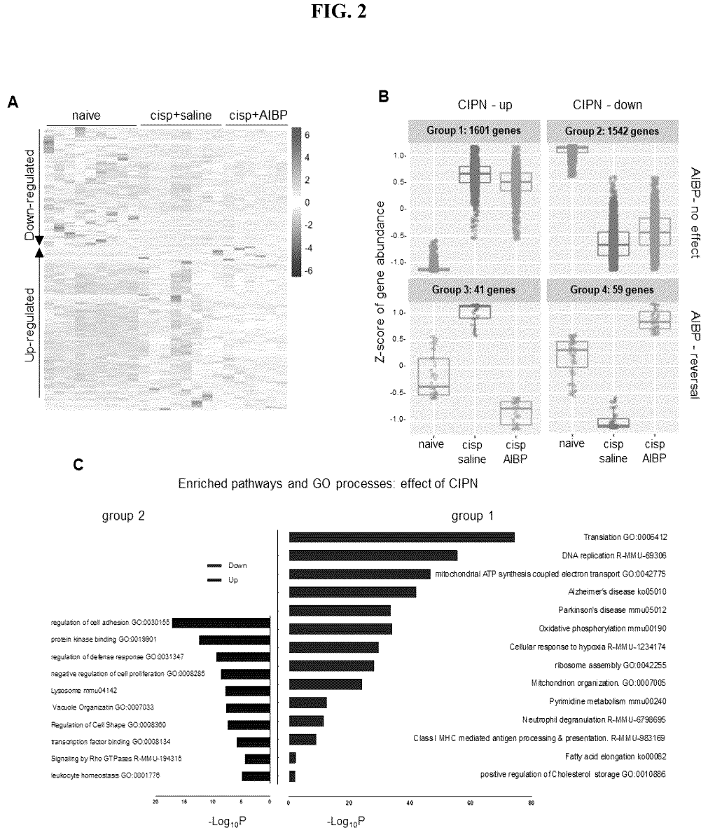

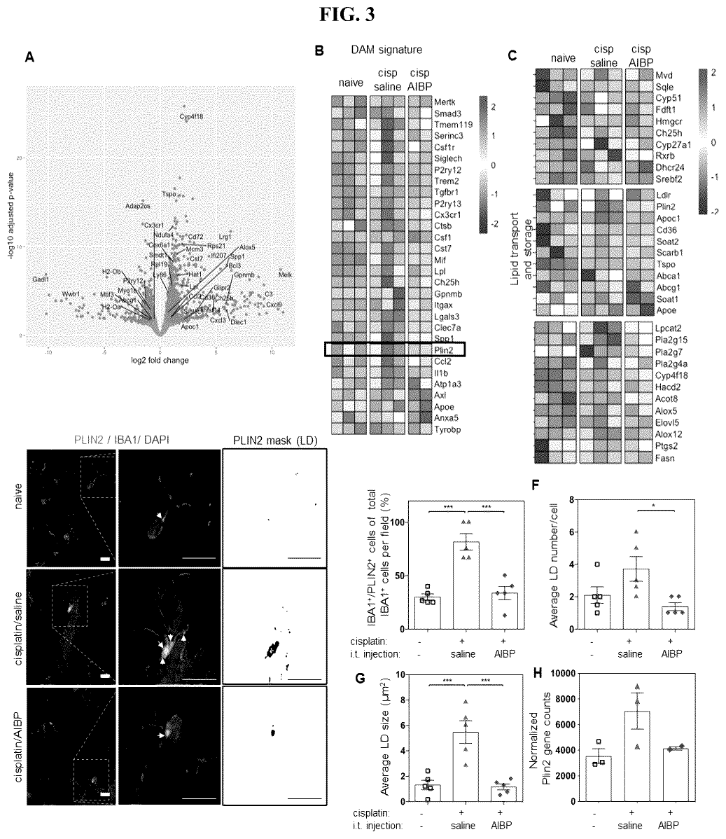

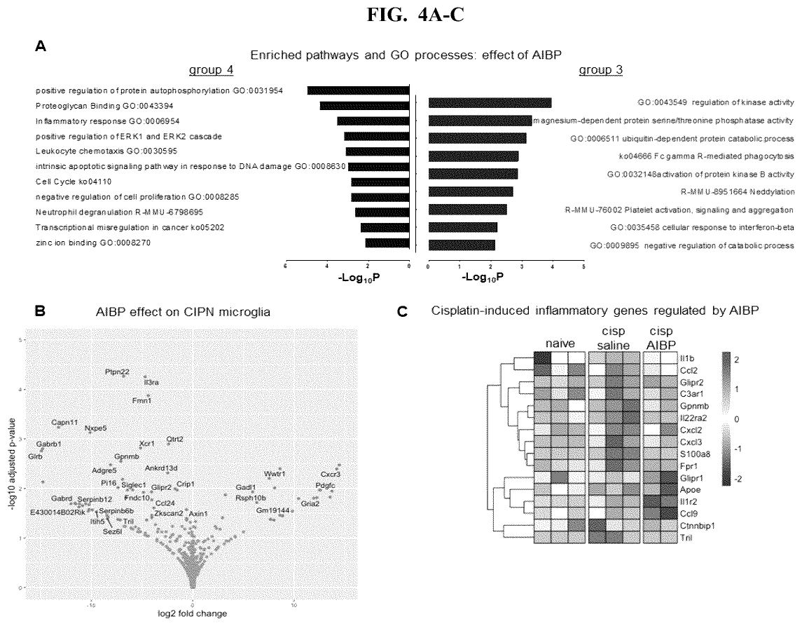

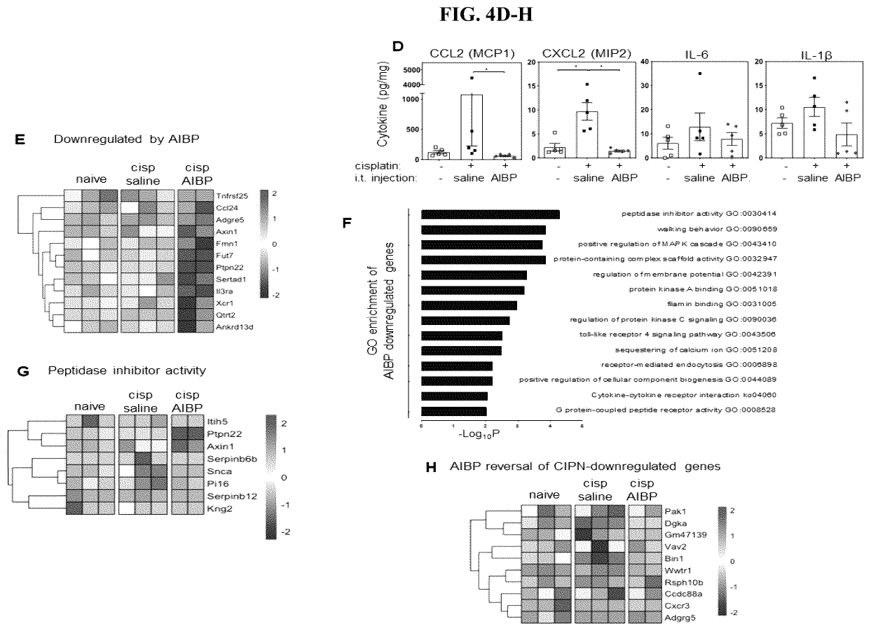

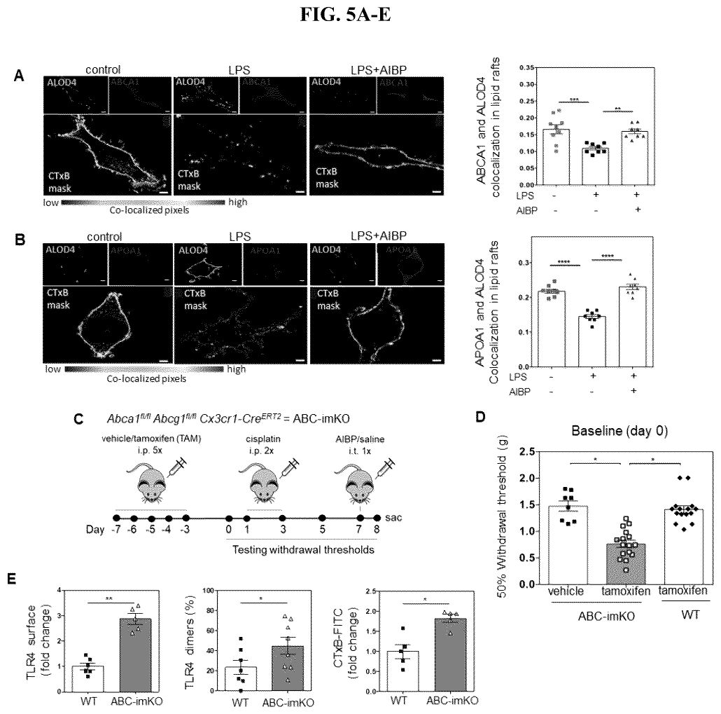

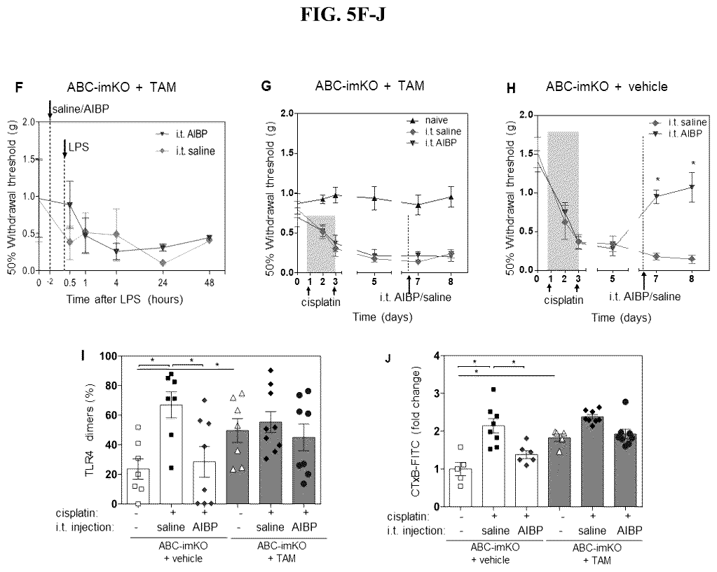

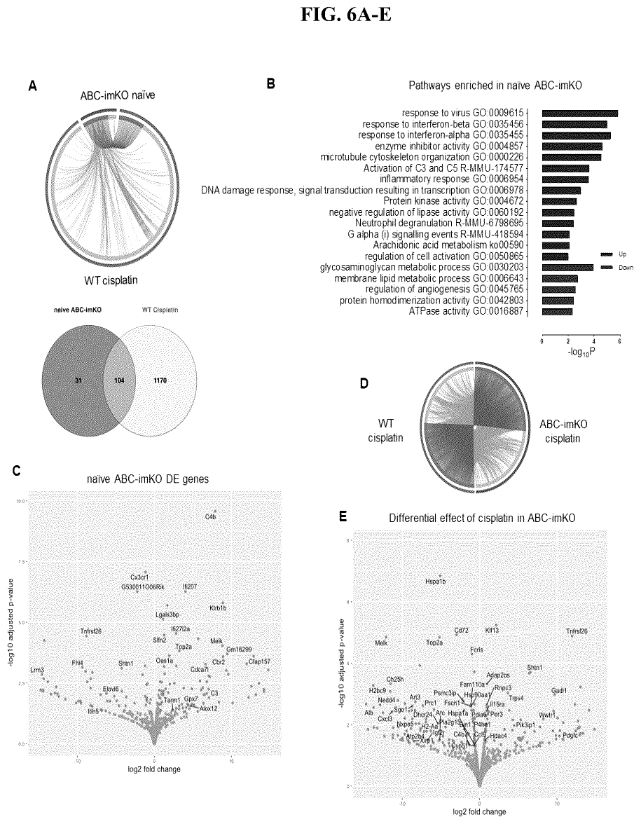

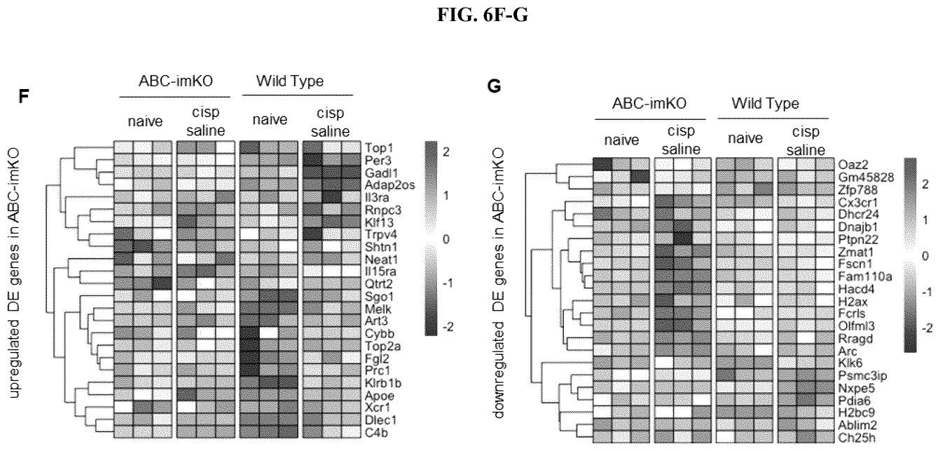

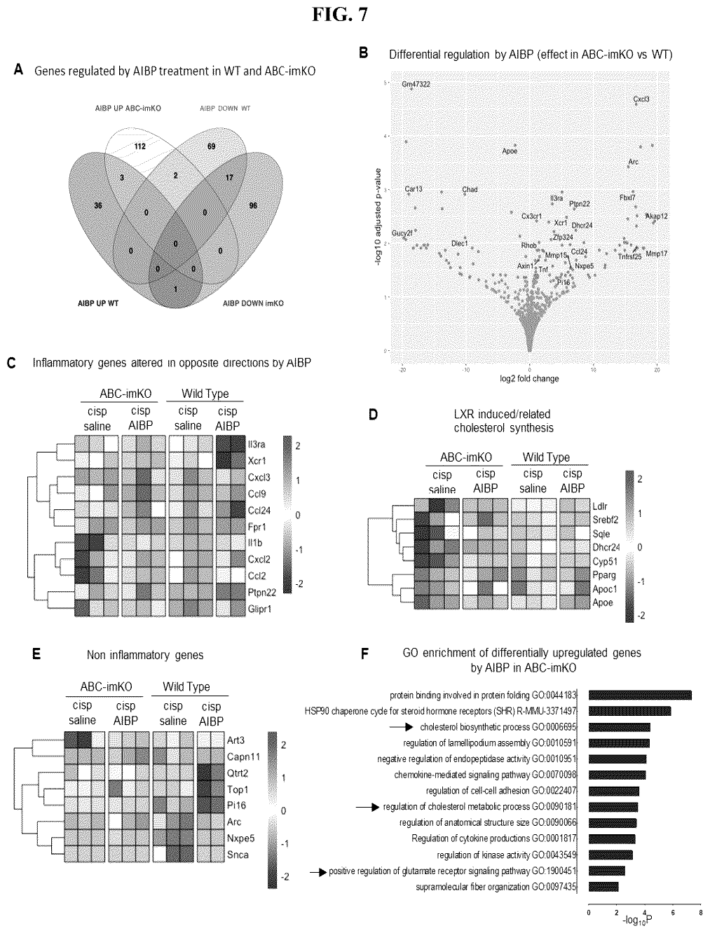

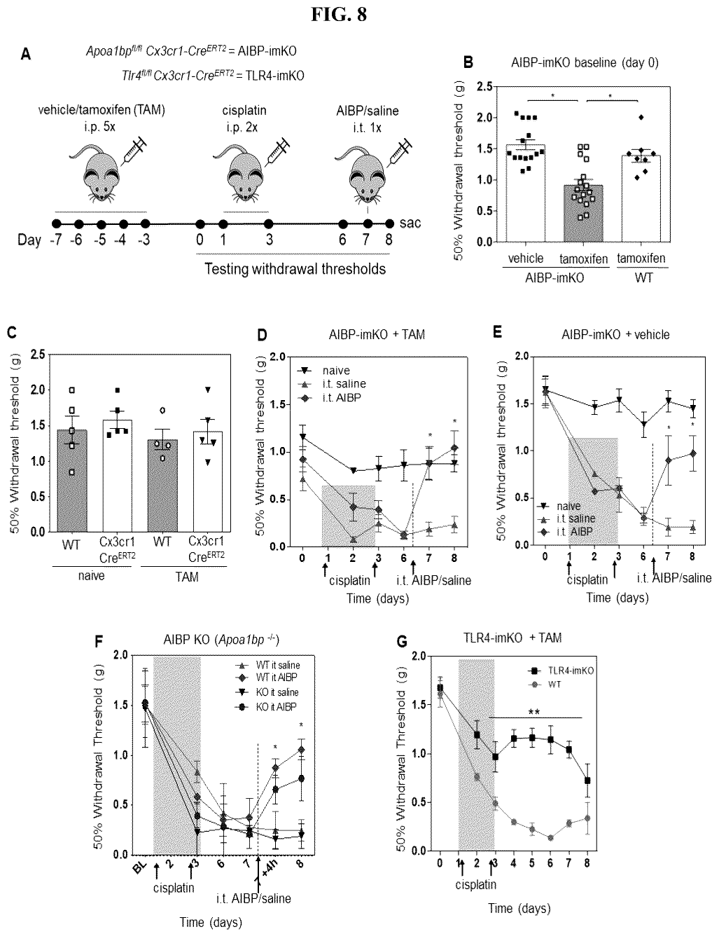

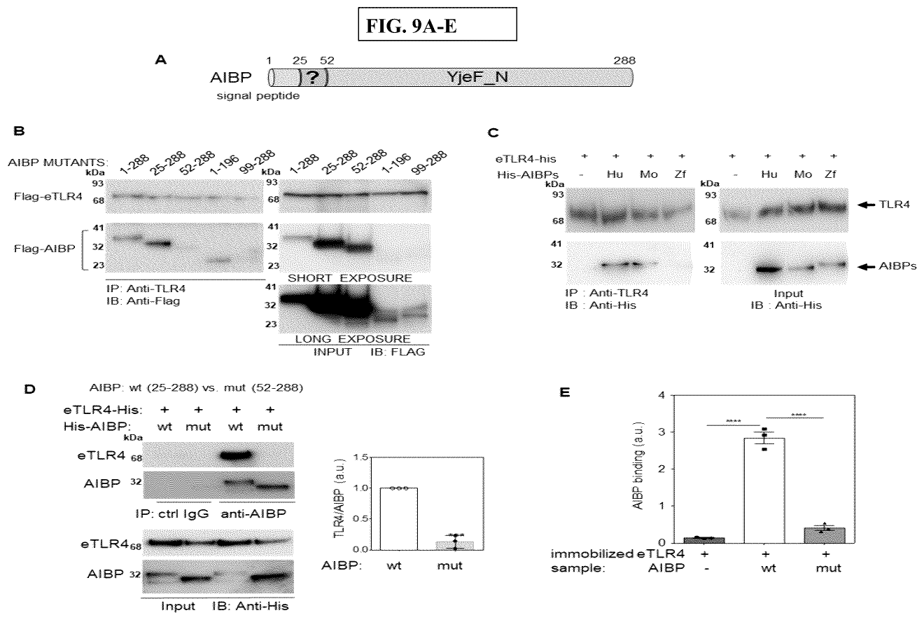

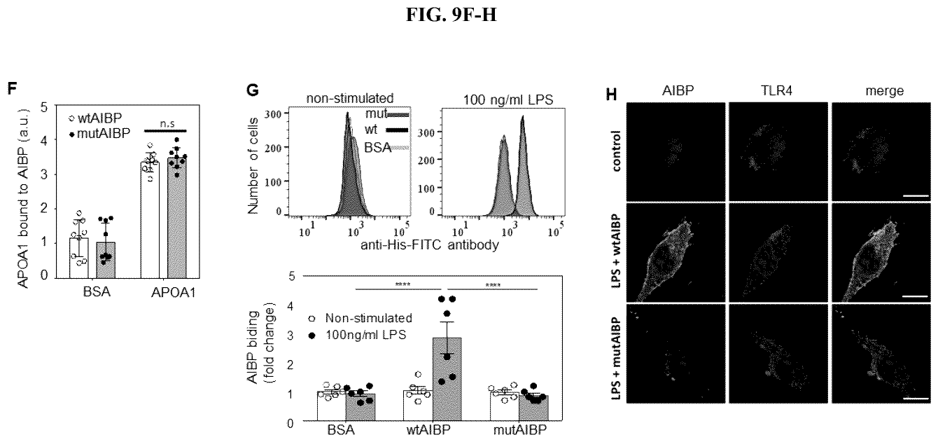

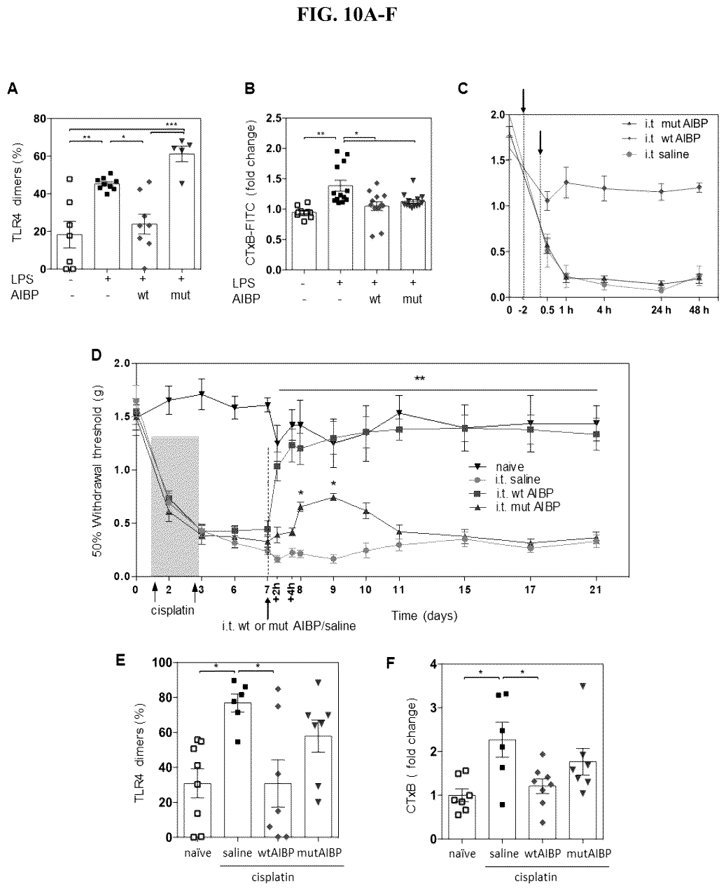

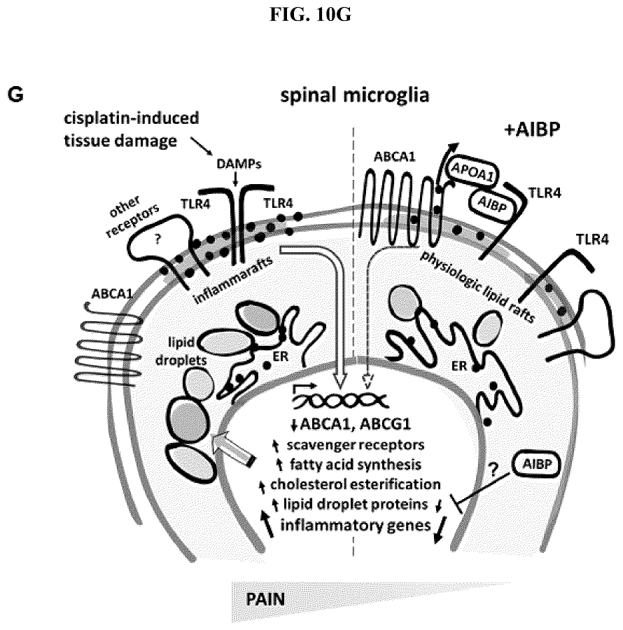

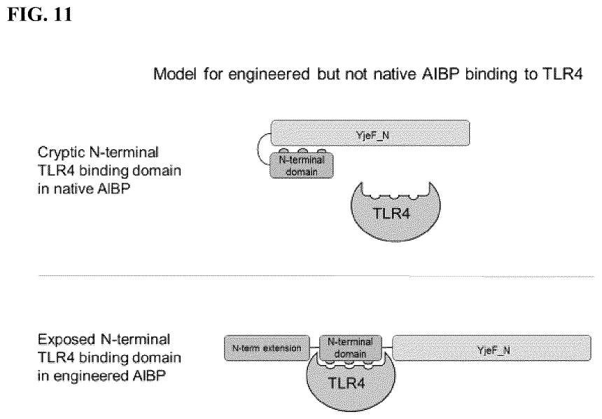

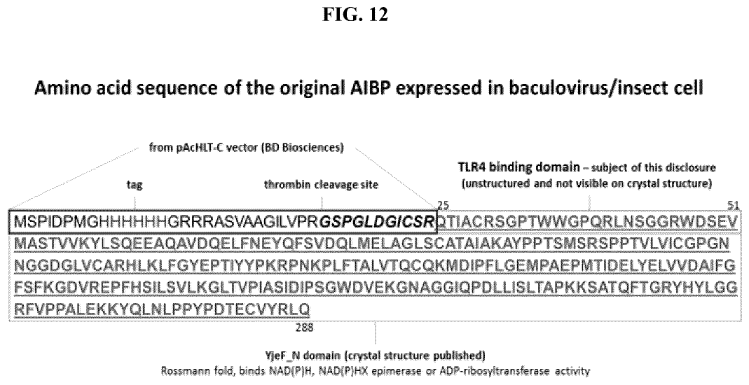

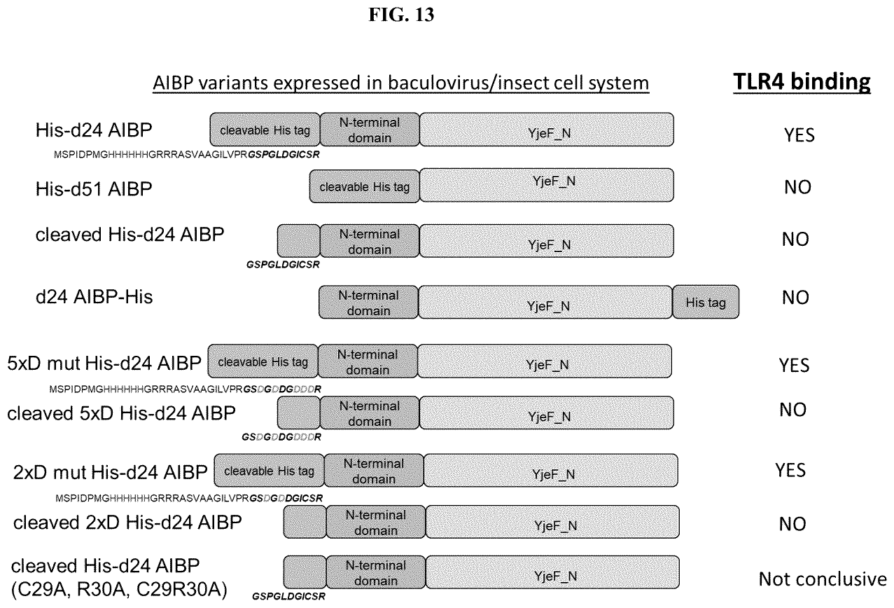

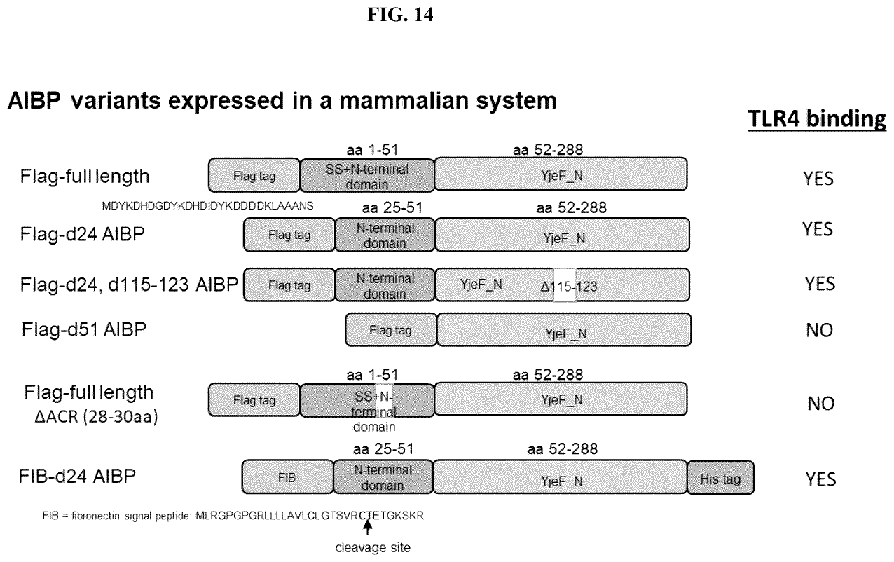

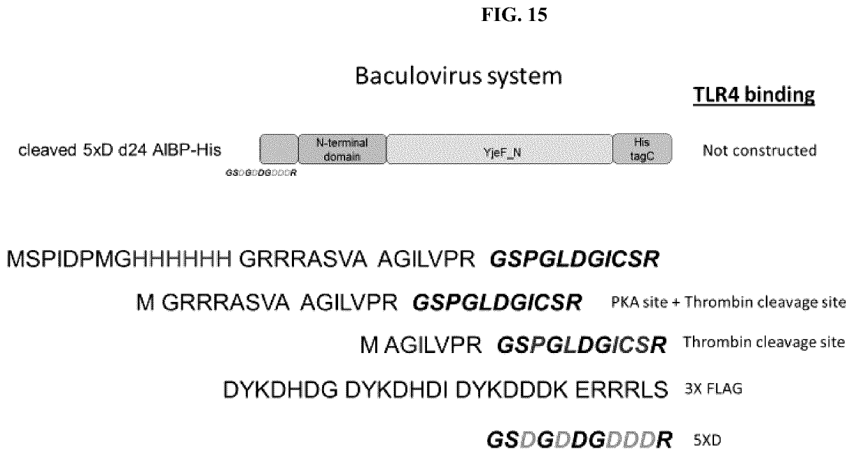

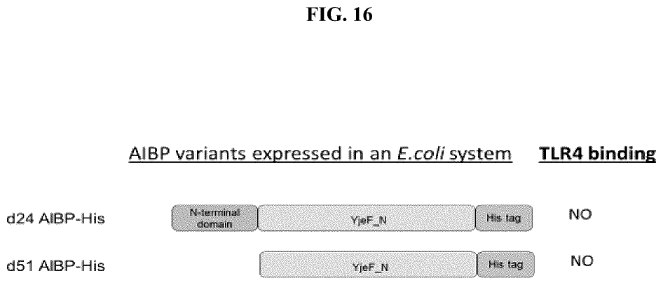

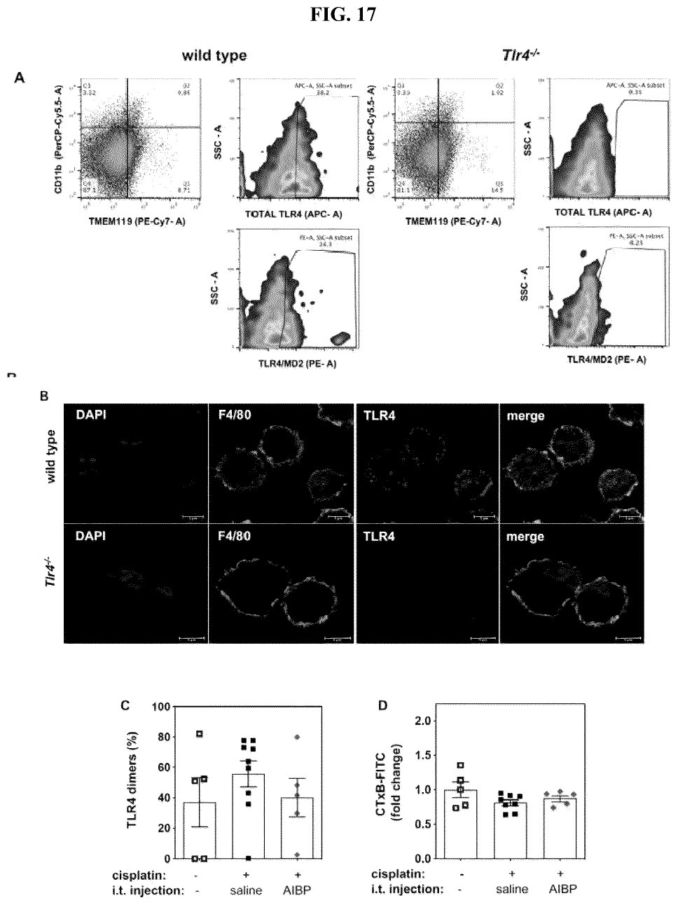

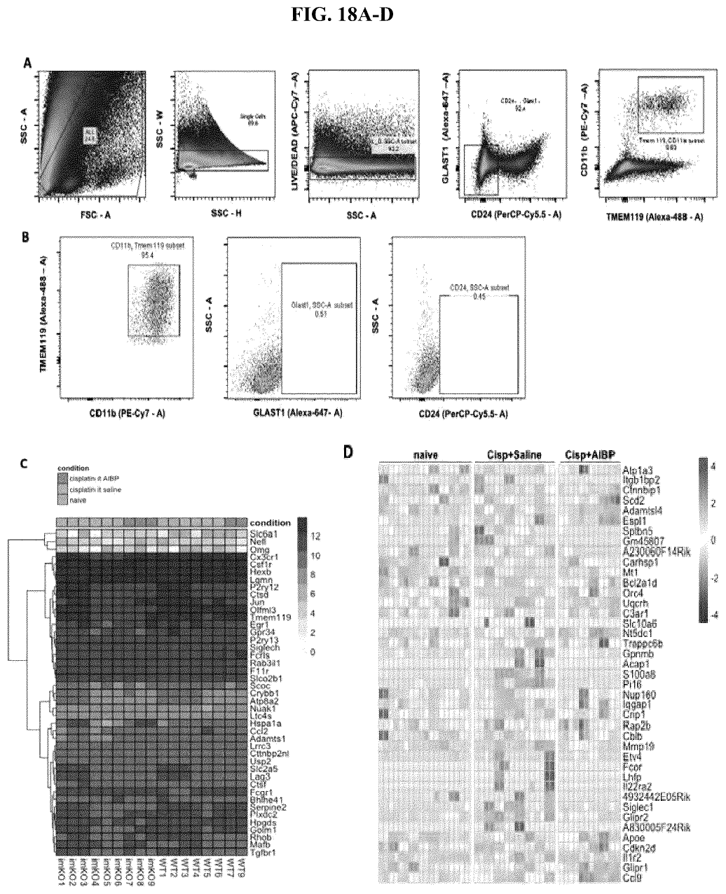

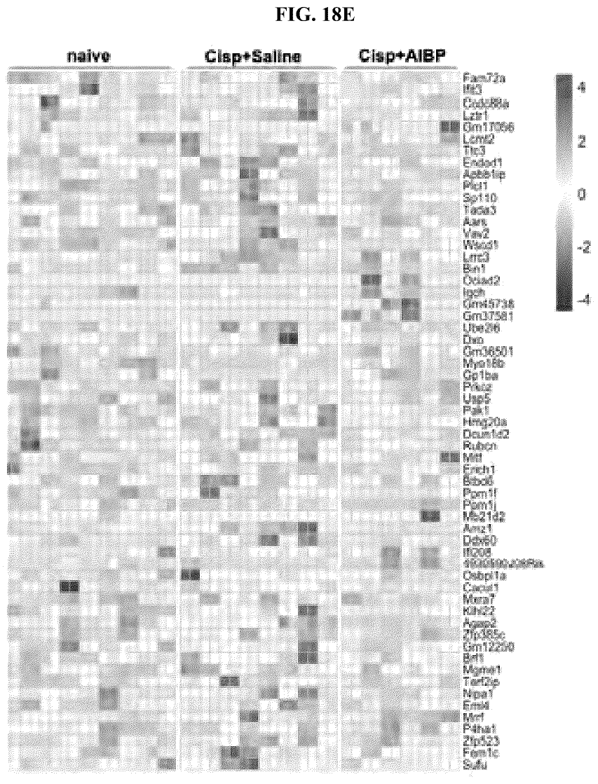

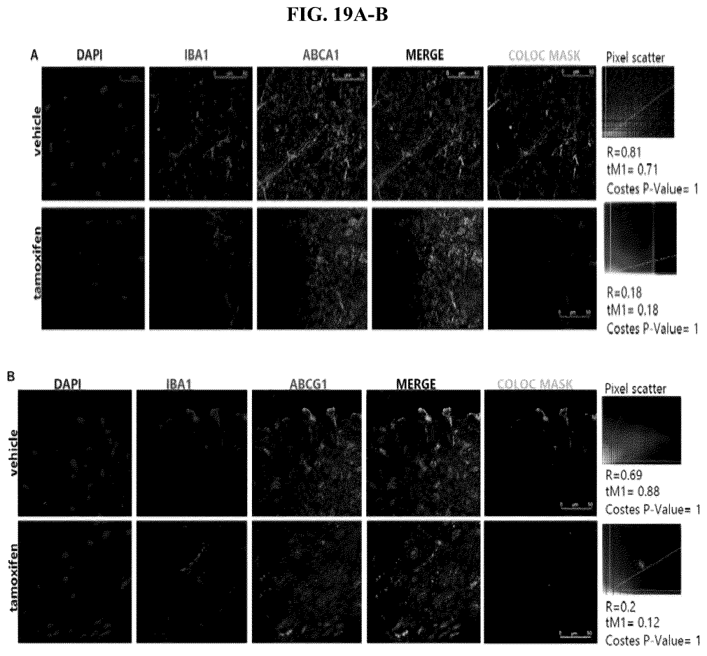

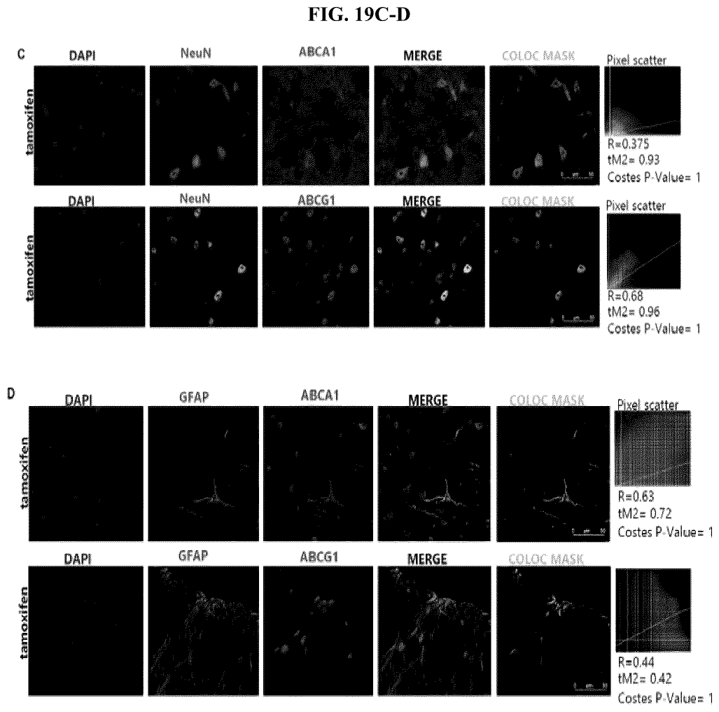

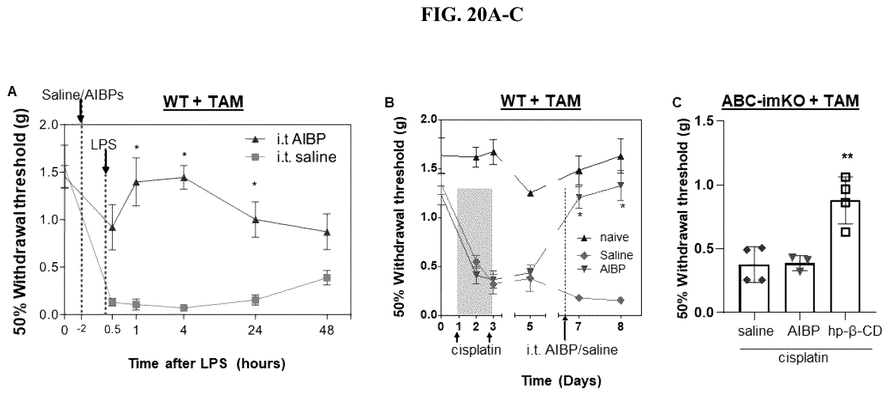

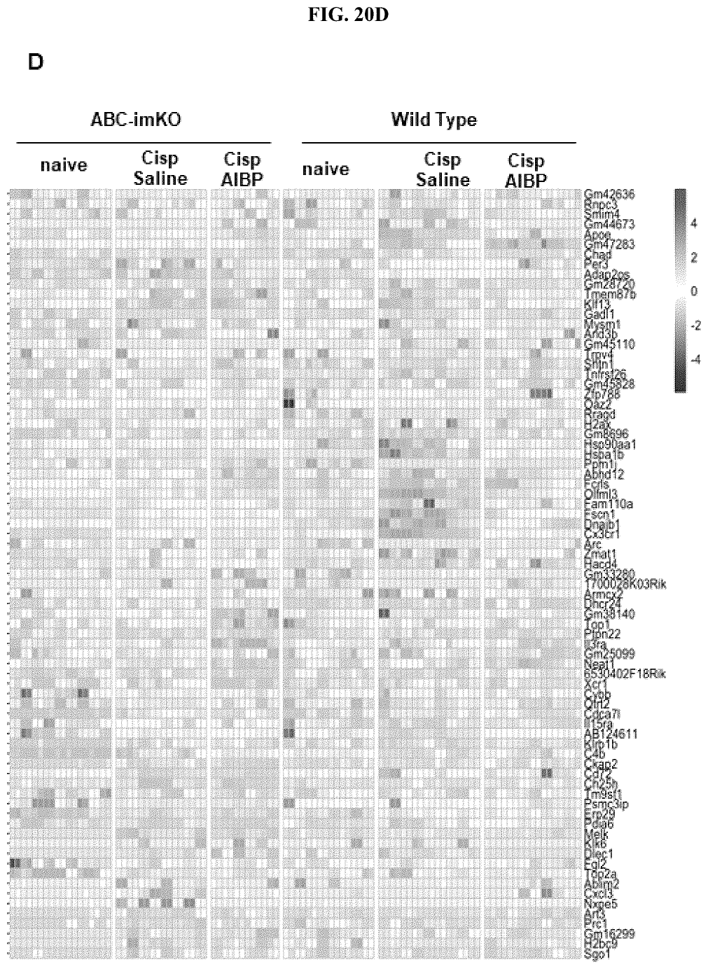

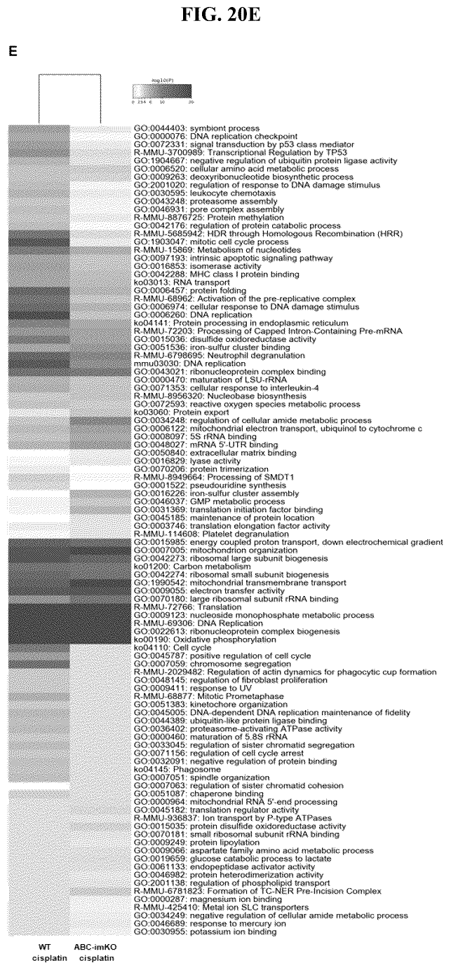

FIG. 1A illustrates images of a heatmap plot of DEGs across all samples;

FIG. 1B graphically illustrates data showing that Groups of significant DEGs were clustered based on expression profile patterns in different treatment conditions; and

FIG. 1C graphically illustrates data showing a pathway and GO enrichment analysis of upregulated (group 1 in right panel) and downregulated (group 2 in left panel) genes induced by cisplatin treatment, upregulated pathways are shown in in right panel “group 1” (red) and downregulated pathways in left panel “group 2” (in blue),

as discussed in detail in Example 1, below.

FIG. 3A-H illustrate data showing disease associated microglia (DAM) and lipid related gene expression and lipid droplets in spinal microglia of CIPN mice:

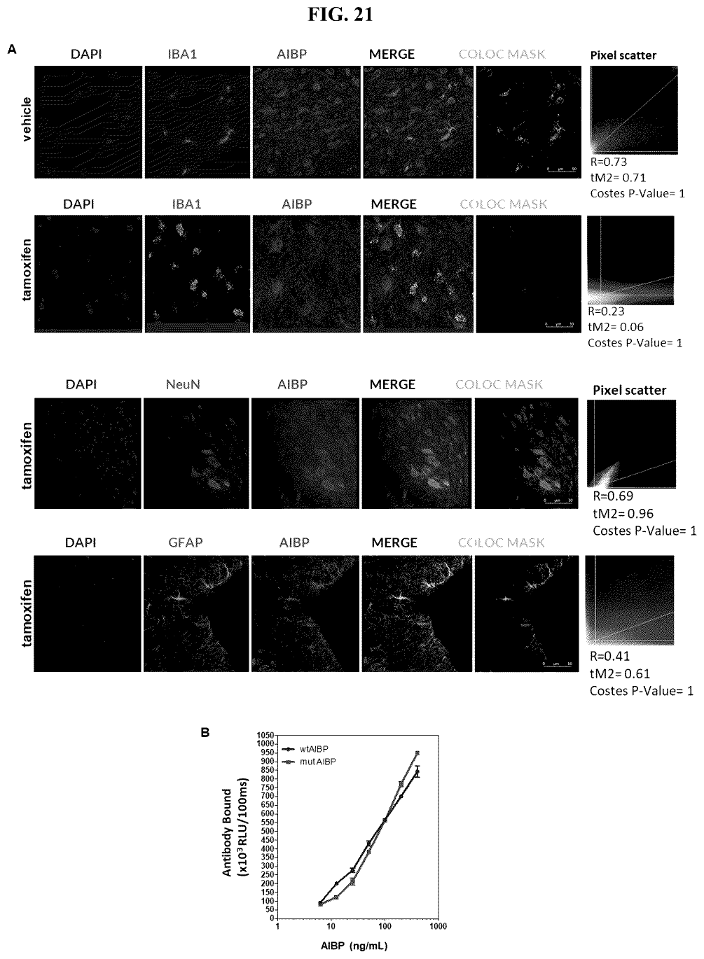

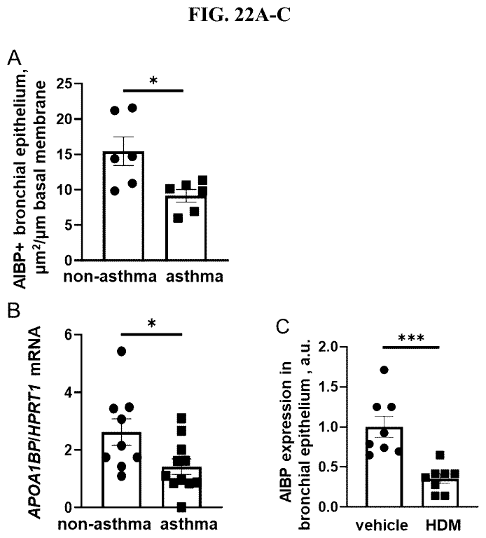

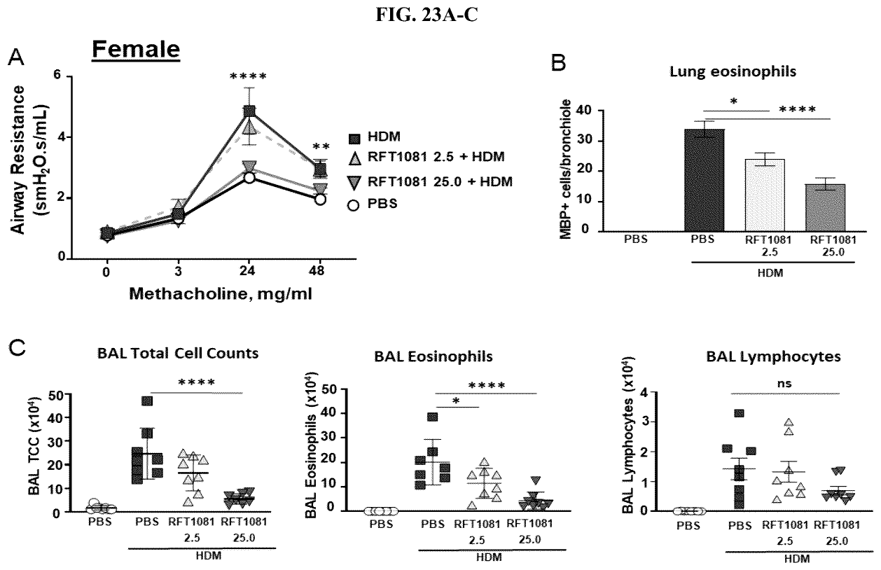

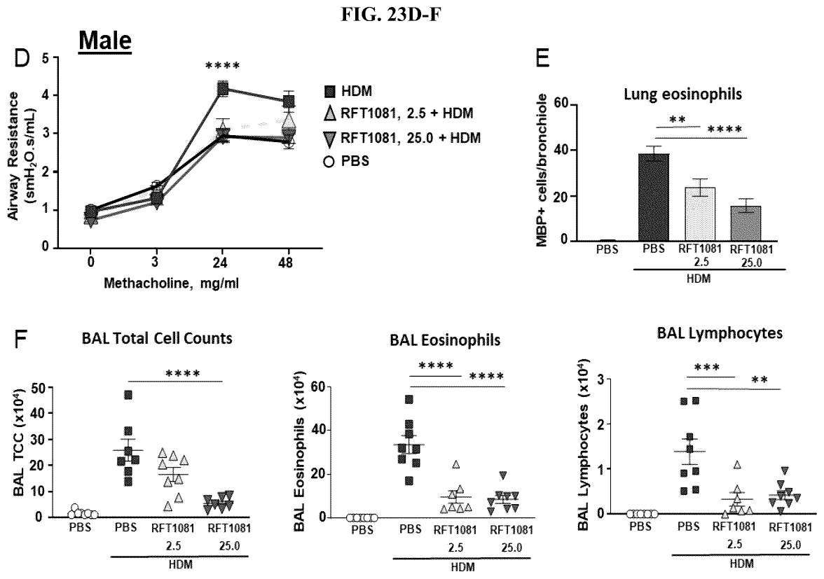

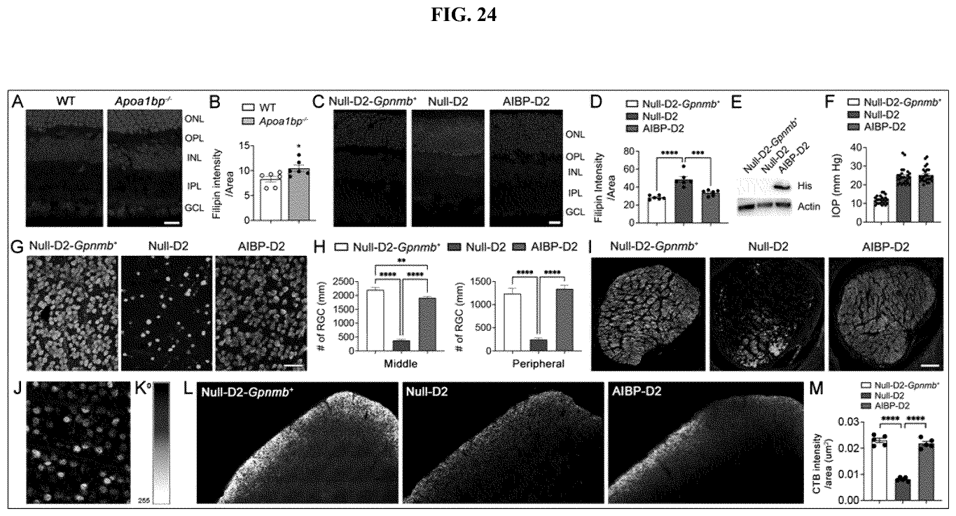

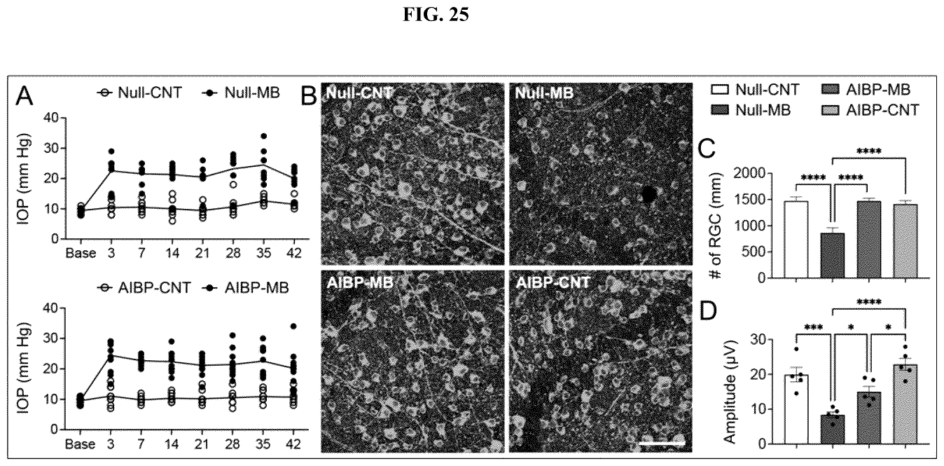

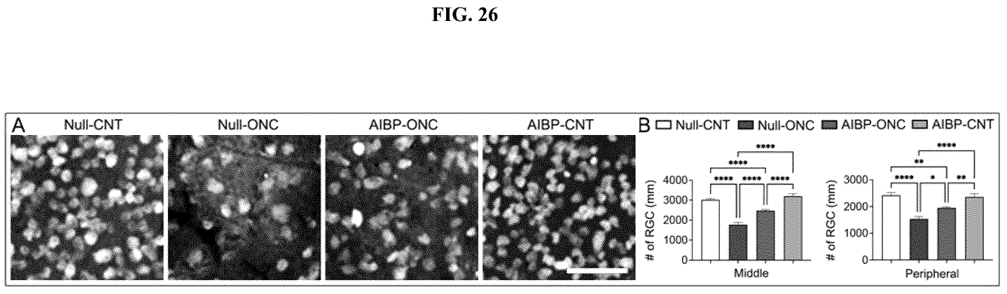

FIG. 3A-C illustrate the same groups as in FIG. 2: FIG. 3A illustrates an image of a volcano plot of upregulated and downregulated genes in spinal microglia of cisplatin-treated vs. naïve mice; FIG. 3B illustrates an image of a heatmap depicting disease associated microglia (DAM) signature genes; FIG. 3B illustrates an image of a heatmap of log 2 normalized gene counts scaled by row showing lipid related gene sets; and

FIG. 3D-H graphically illustrates data showing lipid droplet accumulation in spinal microglia measured by PLIN2 immunostaining in spinal cord sections co-stained with IBA1 and DAPI, with FIG. 3D showing; FIG. 3E graphically illustrating IBA1+/PLIN2+ cells of total IBA1+ cells per field, with or without cisplatin and/or AIBP; FIG. 3F graphically illustrating average LD numbers/cell with or without AIBP; FIG. 3G graphically illustrating average LD size with or without cisplatin and/or AIBP; FIG. 3H graphically illustrating normalized Plin2 gene counts with or without AIPB,

as discussed in detail in Example 1, below.

FIG. 4A-H illustrate data showing gene expression in spinal microglia of CIPN mice, and the effect of AIBP:

FIG. 4A illustrates a pathway and Gene Ontology (GO) enrichment analysis of CIPN-upregulated genes that were downregulated by AIBP (see group 3 in FIG. 2B)) and CIPN-downregulated genes that were upregulated by AIBP (group 4);

FIG. 4B illustrates differentially expressed genes (DEGs) in spinal microglia induced by i.t. AIBP, a volcano plot of up and down regulated genes in cisplatin/AIBP versus (vs.) cisplatin/saline treated mice;

FIG. 4C illustrates a heatmap of inflammatory genes in group 3 upregulated in CIPN and downregulated by AIBP;

FIG. 4D graphically illustrates data showing cytokine protein expression in spinal tissue from WT naïve, cisplatin/saline and cisplatin/AIBP groups;

FIG. 4E illustrates a heatmap of inflammatory genes not induced by cisplatin but downregulated by AIBP;

FIG. 4F graphically illustrates a pathway and GO enrichment analysis of all genes downregulated by AIBP;

FIG. 4G illustrates a heatmap of non-inflammatory genes downregulated by AIBP included in the most enriched pathway: peptidase inhibitor activity pathway; and

FIG. 4H illustrates a heatmap of genes whose downregulation in CIPN was reversed by AIBP,

as discussed in detail in Example 1, below.

FIG. 5A-J illustrate data showing that ABCA1 and ABCG1 expression in microglia controls nociception and is required for AIBP-mediated reversal of allodynia in a mouse model of CIPN:

FIG. 5A-B illustrates data showing data from BV-2 cells incubated for 30 min with AIBP (0.2 μg/mL) or vehicle in complete media, followed by a 5 min incubation with LPS (100 ng/mL), showing colocalization of accessible cholesterol with ABCA1 (FIG. 5A) and APOA1 (FIG. 5B) in lipid rafts;

FIG. 5C schematically illustrates an exemplary experimental design and timeline of tamoxifen, cisplatin, AIBP or saline injection in mice;

FIG. 5D graphically illustrates data showing baseline (day 0) withdrawal thresholds before the start of cisplatin intervention;

FIG. 5E graphically illustrates data showing TLR4 surface expression and dimerization and lipid rafts (CTxB) in CD11b⁺TMEM119⁺ spinal microglia of naïve WT and ABC-imKO mice at baseline (day 0) (n=5 for TLR4 surface expression and lipid raft content analysis for both groups;

FIG. 5F graphically illustrates data showing withdrawal thresholds after i.t. saline or AIBP (0.5 μg/5 μl), followed by i.t. LPS (0.1 μg/5 μl) in TAM-induced ABC-imKO mice;

FIG. 5G-H graphically illustrate data showing withdrawal thresholds following i.p. cisplatin and i.t. saline or AIBP (0.5 μg/5 μl) injections in TAM-induced ABC-imKO (FIG. 5G) and non-induced (vehicle) ABC-imKO (FIG. 5H) mice;

FIG. 5I-J graphically illustrate data showing TLR4 dimerization (FIG. 5I) and lipid rafts (FIG. 5J) in CD11b⁺TEMEM119⁺ spinal microglia at day 8 in the groups shown in panels FIG. 5G and FIG. 5H, as discussed in detail in Example 1, below.

FIG. 6A-G illustrate data showing expression in spinal microglia of ABC-imKO mice:

FIG. 6A top image schematically illustrates overlapping genes and pathways induced in naïve ABC-imKO microglia and shared with WT microglia in mice treated with cisplatin, showed in purple (darker, upper) lines connecting overlapping genes and in blue (lighter, lower) lines connecting the overlapping enriched pathways, FIG. 6A bottom image is a Venn diagram of upregulated genes in spinal microglia from WT cisplatin and ABC-imKO naïve mice;

FIG. 6B illustrates an enrichment pathway analysis of up and down regulated genes induced by ABCA1 and ABCG1 knockdown in microglia;

FIG. 6C illustrates DEGs in naïve spinal microglia of TAM-induced ABC-imKO mice;

FIG. 6D schematically illustrates overlapping genes and pathways induced by cisplatin treatment in ABC-imKO microglia and shared with WT microglia in mice treated with cisplatin;

FIG. 6E illustrates DEGs in spinal microglia of cisplatin-treated, TAM-induced ABC-imKO mice compared to cisplatin-treated WT mice; and

FIG. 6F-G illustrate a heatmap of DEGs upregulated genes (FIG. 6F) or downregulated genes (FIG. 6G) in ABC-imKO microglia either in naïve or cisplatin condition, as discussed in detail in Example 1, below.

FIG. 7A-F illustrate data showing that microglial reprogramming by AIBP is dependent on ABCA1/ABCG1 expression:

FIG. 7A schematically illustrates a Venn diagram comparing the effect of AIBP treatment on gene expression in WT and ABC-imKO mice in which CIPN was induced by cisplatin;

FIG. 7B schematically illustrates a Volcano plot representation of up and down regulated genes by AIBP treatment in CIPN comparing AIBP effect on ABC-imKO vs. WT mice;

FIG. 7C schematically illustrates a heatmap of log 2 normalized gene counts of inflammatory genes altered by AIBP in an ABC-dependent manner (downregulated by AIBP in WT microglia but upregulated by AIBP in ABC-imKO;

FIG. 7D schematically illustrates a heatmap of cholesterol synthesis and LXR related genes comparing cisplatin and AIBP effect in wild type and ABC-imKO;

FIG. 7E schematically illustrates a heatmap of non-inflammatory genes regulated by AIBP in an ABC-dependent manner;

FIG. 7F schematically illustrates an enrichment pathway analysis of upregulated genes by AIBP in ABC-imKO microglia, as discussed in detail in Example 1, below.

FIG. 8A-G illustrate that endogenous AIBP and TLR4 in microglia are important in nociception:

FIG. 8A schematically illustrates an exemplary experimental design and timeline: Tamoxifen; cisplatin; AIBP; and/or saline are injected;

FIG. 8B graphically illustrates baseline (day 0 in FIG. 8A) withdrawal thresholds before the start of cisplatin intervention;

FIG. 8C graphically illustrates WT and Cx3cr1-Cre^{ERT2} (no floxed genes) mice were tested for withdrawal threshold before (naïve, day −7 in panel A timeline) and after (TAM, day 0) tamoxifen injection regimen;

FIG. 8D-F graphically illustrate withdrawal thresholds following i.p. cisplatin and i.t. saline or AIBP injections in: (FIG. 8D) TAM-induced AIBP-imKO mice; non-induced (vehicle) AIBP-imKO mice (FIG. 8E); and bred in-house whole body AIBP knockout mice (FIG. 8F); and

FIG. 8G graphically illustrates withdrawal thresholds in WT and tamoxifen-induced TLR4-imKO mice following cisplatin injections,

as discussed in detail in Example 1, below.

FIG. 9A-H illustrates data showing the identification of the domain in the AIBP molecule responsible for TLR4 binding:

FIG. 9A schematically illustrates human AIBP with signal peptide, amino acids (aa) 1-24, previously uncharacterized N-terminal domain (aa 25-51), and YjeF_N domain (aa 52-288);

FIG. 9B illustrates an image of a PAGE separation of flag-tagged deletion mutants of human AIBP, which were co-expressed in HEK293 cells with the Flag-tagged TLR4 ectodomain (eTLR4); cell lysates were immunoprecipitated (IP) with an anti-TLR4 antibody and immunoblotted (IB) with an anti-Flag antibody;

FIG. 9C illustrates an image of a PAGE separation of his-tagged human (hu), mouse (mo) and zebrafish (zf) AIBP, all lacking the signal peptide, expressed in a baculovirus/insect cell system, and were combined in a test-tube with eTLR4-His, followed by IP with an anti-TLR4 antibody and IB with an anti-His antibody;

FIG. 9D-H illustrate data showing binding of His-tagged wild type (wt, 25-288 aa) and the deletion mutant (mut, 52-288 aa) human AIBP to eTLR4, APOA1 and microglia, and immunoprecipitation (IP) of eTLR4 and wtAIBP or mutAIBP in a test tube with an anti-AIBP antibody, blot and quantification from 3 independent experiments:

FIG. 9D illustrates (left image) a PAGE separation, where ELISA were done with plates coated with eTLR4 and incubated with wtAIBP or mutAIBP, the right image graphically shows the amounts of TLR4/AIBP for wt and mu AIPB;

FIG. 9E graphically illustrates AIBP binding on immobilized eTLR4 with wt or mut AIBP (or no AIBP), where ELISA werre done with plates coated with BSA, wtAIBP or mutAIBP and incubated with APOA1;

FIG. 9F graphically illustrates APOA1 bound to AIBP;

FIG. 9G graphically illustrates number of cells with APOA1 bound to AIBP using flow cytometry (upper graphs), and (lower graph) AIBP binding (fold change) to wt and mut AIBP in non-stimulated LPS stimulated cells;

FIG. 9H illustrates APOA1 bound to AIBP using confocal imaging, showing binding of wtAIBP and mutAIBP to BV-2 microglia cells, unstimulated or treated for 15 min with LPS,

as discussed in detail in Example 1, below.

FIG. 10A-G illustrate data showing that intrathecal delivery of AIBP lacking the TLR4 binding domain cannot alleviate CIPN allodynia:

FIG. 10A-B graphically illustrate TLR4 dimerization (FIG. 10A) and lipid rafts (FIG. 10B) in BV-2 cells pre-treated with wt AIBP or mut AIBP and stimulated with LPS;

FIG. 10C graphically illustrates withdrawal thresholds in WT mice that received i.t. AIBP (0.5 μg/5 μL) or saline (5 μL), followed by i.t. LPS;

FIG. 10D graphically illustrates withdrawal thresholds in WT mice in response to i.p. cisplatin, followed by i.t. wtAIBP, mutAIBP or saline;

FIG. 10E-F graphically illustrate TLR4 dimerization (FIG. 10E) and lipid rafts (FIG. 10F) in CD11b⁺/TMEM119⁺ microglia from lumbar spinal cord of mice in experimental groups shown in panel FIG. 10D, at day 21;

FIG. 10G schematically illustrates a diagram illustrating the effect of chemotherapeutic-induced peripheral neuropathy (CIPN) using cisplatin-induced tissue damage (damage-associated molecular patterns (DAMPs)) and AIBP treatment on microglia gene expression and lipid droplet accumulation, black dots in the plasma membrane and the ER depict cholesterol,

as discussed in detail in Example 1, below.

FIG. 11 schematically illustrates a model of unfolding or exposing a cryptic N-terminal domain in the AIBP molecule; the diagram summarizes and illustrates results of experiments shown in FIGS. 12-14, which demonstrate that in native AIBP the N-terminal domain (green) is hidden or cryptic or not sufficiently exposed to mediate AIBP binding to TLR4 (top panel), extending the N-terminus with additional amino acids (orange) changes the AIBP conformation and makes the N-terminal domain of AIBP (green) accessible for TLR4 binding (bottom panel).

FIG. 12 illustrates an exemplary amino acid sequence of an engineered AIBP, as provided herein (SEQ ID NO:35): the amino acid sequence of an extended AIBP molecule depicted in the bottom panel of FIG. 11, blue letters, amino acids from the native AIBP sequence; green box, the TLR4-binding sequence (amino acids 25-51 of the human AIBP sequence); black letters and (red) box, added amino acids.

FIG. 13 schematically illustrates TLR4 binding of various exemplary engineered forms of AIBP: all proteins were expressed and purified from a baculovirus/insect cell system:

His-d24AIBP: corresponds to the amino acid sequence shown in FIG. 12, the amino acid sequence shows the sequence of the orange box “cleavable His tag”,

all other drawings show different modifications and corresponding changes in the amino acid sequence introduced to the AIBP molecule, the green “N-terminal domain” box depicts the amino acid 25-51 sequence of native AIBP, the column on the right shows the results of co-immunoprecipitation experiments of the AIBP variants with a recombinant ectodomain of TLR4,

for His-24 AIBP: MSPIDPMGHHHHHHGRRRASVAAGILVPRGSPGLDGICSR (SEQ ID NO:2)

for “cleaved His-d24 AIBP”, GSPGLDGICSR (SEQ ID NO:9),

for “5×D mut His-d24 AIPB”: MSPIDPMGHHHHHHGRRRASVAAGILVPRGSDGDDGDDDR (SEQ ID NO:19),

for “cleaved 5×D His-d24 AIPB” GSDGDDGDDDR (SEQ ID NO:10),

for “2×D mut His-d24 AIBP MSPIDPMGHHHHHHGRRRASVAAGILVPRGSDGDDGICSR (SEQ ID NO:11), and

for “cleaved His-d24 AIBP” GSPGLDGICSR (SEQ ID NO:9).

FIG. 14 schematically illustrates TLR4 binding of various engineered forms of AIBP: all proteins were co-expressed with the full-length TLR4 in a mammalian system: SS, secretion signal, corresponding to the amino acids 1-24 in the human AIBP sequence; the column on the right shows the results of co-immunoprecipitation from cell lysates of the AIBP variants with TLR4,

MDYKDHKGKYKDHDIDYKDDDDKLAAANS for “Flag-full length” (SEQ ID NO:14), and

for the fibronectin signal peptide MLRGPGPGRLLLLAVLCLGTSVRCTETGKSKR (SEQ ID: NO:24).

FIG. 15 schematically illustrates various AIBP constructs to optimize the structure for TLR4 affinity: baculovirus/insect cell expression system:

(SEQ ID NO: 11)
GSDGDDGDDDR,
(SEQ ID NO: 2)
MSPIDPMGHHHHHHGRRRASVAAGILVPRGSPGLDGICSR
for “PKA site + Thrombin cleavage site”:
(SEQ ID NO: 17)
MGRRRASVAAGILVPRGSPGLDGICSR
for “thrombin cleavage site”
(SEQ ID NO: 18)
MAGILVPRGSPGLDGICSR
for “3x FLAG”
(SEQ ID NO: 11)
GSDGDDGDDDR,
for “5XD”.

FIG. 16 schematically illustrates TLR4 binding of various engineered forms of AIBP: all proteins were expressed and purified from an E. coli, the column on the right shows the results of co-immunoprecipitation experiments of the AIBP variants with a recombinant ectodomain of TLR4.

FIG. 17A-D provides validation of the specificity of TLR4 antibodies used for flow cytometry and microscopy, and also shows TLR4 dimerization and lipid rafts measured in dorsal root ganglia macrophages:

FIG. 17A graphically illustrates flow cytometry of single cell suspensions from spinal cords of WT (left images) and Tlr4−/− mice (right images) showing TLR4-APC and TLR4/MD2-PE antibodies staining of CD11b+(PercP-Cy5.5)/TMEM199+(Pe-Cy7) microglia;

FIG. 17B illustrates confocal images of peritoneal elicited macrophages from WT and Tlr4−/− mice co-stained with F4/80-FITC and TLR4-647 antibodies; Scale bar, 5 μm; and

FIG. 17C-D graphically illustrate flow cytometry analysis of CD11b+ DRG macrophages cells showing TLR4 dimerization (FIG. 17C) and lipid raft content measured by CTxB staining (FIG. 17D) 24 hours after i.t. saline or AIBP,

as discussed in Example 1, below.

FIG. 18A-E (or, FIG. S2, or supplementary FIG. 2) shows FACS sorting strategy for spinal microglia, quality controls and phenotypic controls for RNA-seq:

FIG. 18A illustrates sorting strategy for lumbar CD11b+TMEM119+ spinal microglia, including: SSC-A and FSC-A, SSC-W and SSC-H, UVE/DEAD (APC-Cy7-A) and SSC-A, GLAST1 and CD24, and, CD11b and TMEM119;

FIG. 18B illustrates flow cytometry analysis of sorted microglia measuring purity of sorted cells and absence of GLAST1+ astrocytes or CD24+ neurons, including TMEM119 and CD11b, SSC-A and GLAST1, and SSC-1 and CD24;

FIG. 18C illustrates microglial linage analysis with a heatmap of microglia specific genes; and

FIG. 18D-E illustrate heatmaps of CIPN-repressed genes that were up-regulated by AIBP (group 4) (FIG. 18D) and CIPN-induced genes that were downregulated by AIBP (group 3) in wildtype mice (FIG. 18E);

as discussed in Example 1, below.

FIG. 19A-D provides immunohistochemical validation of conditional knockout of ABCA1 and ABCG1 in spinal microglia of tamoxifen-induced ABC-imKO mice:

FIG. 19A illustrates DAPI, IBA1, ABCA1, MERGE, and COLOC MASK, with and without tamixifen

FIG. 19B illustrates DAPI, IBA1, ABCG1, MERGE, and COLOC MASK, with and without tamixifen,

FIG. 19C illustrates DAPI, NeuN, ABCA1, MERGE, and COLOC MASK, with and without tamixifen, and

FIG. 19D illustrates DAPI, GFAP, ABCA1, MERGE, and COLOC MASK, with and without tamixifen, as discussed in further detail in Example 1, below.

FIG. 20A-E show tactile allodynia data for tamoxifen-treated WT mice in i.t. LPS and CIPN experiments, and provide additional RNA-seq data for ABC-imKO dependent genes and the cisplatin effect on ABC-imKO vs. WT mice:

FIG. 20A-B graphically illustrate data where, as a control for ABC-imKO mice, inhouse bred WT littermate mice were subjected to the tamoxifen regimen (TAM, 200 μL/day, 10 mg/mL, 5 consecutive days), followed by (FIG. 20A) i.t. injection of AIBP (0.5 μg/5 μL) or saline (5 μL) and i.t. LPS (0.1 μg/5 μL) 2 hours later; and (FIG. 20B) i.p. injections of cisplatin (2.3 mg/Kg) on day 1 and day 3 followed by i.t. injection of AIBP (0.5 μg/5 μL) or saline (5 μL) on day 7;

FIG. 20C graphically illustrate data where ABC-imKO mice were injected with TAM and then cisplatin as above, followed by i.t. saline (5 μL), AIBP (0.5 μg/5 μL) or hp-β-CD (0.25 mg/5 μL) on day 7;

FIG. 20D illustrates a heatmap of differentially regulated genes across all conditions (naïve, induced by cisplatin/saline or cisplatin/AIBP) regulated in an ABC-imKO manner;

FIG. 20D illustrates all significant genes from likelihood ratio test using a reduced model without interaction term (condition: genotype);

FIG. 20E illustrates a heatmap of pathway enrichment of cisplatin upregulated genes in WT and ABC-imKO microglia using cutoff P<0.05, enrichment>1.5 and a minimum overlap of 3 genes in the pathway,

as discussed in further detail in Example 1, below.

FIG. 21A-B provides immunohistochemical validation of AIBP knockout in spinal microglia of tamoxifen-induced AIBP-imKO mice, and demonstrates that the BE-1 monoclonal antibody has similar affinity to wtAIBP and mutAIBP:

FIG. 21A illustrates images of IHC of spinal cord frozen sections from vehicle and tamoxifen induced AIBP-imKO mice, showing colocalization of AIBP staining with IBA1 (microglia), NeuN (neurons) and GFAP (astrocytes);

FIG. 21B graphically illustrates data of a sandwich ELISA using BE-1 as a capture antibody in a microtiter plate, dose response curves to wtAIBP and mutAIBP were detected using a rabbit polyclonal anti-AIBP antibody,

as discussed in further detail in Example 1, below.

FIG. 22A-C graphically illustrate reduced AIBP expression in bronchial epithelium:

FIG. 22A graphically illustrates AIBP+ bronchial epithelium in non-asthma and asthma samples;

FIG. 22B graphically illustrates APOA1BP/HPRT1 mRNA in non-asthma and asthma samples;

FIG. 22C graphically illustrates AIBP expression in bronchial epithelium,

as discussed in further detail in Example 3, below.

FIG. 23A-F graphically illustrate that Compound 7 reduces airway hyper-responsiveness and eosinophilic pulmonary inflammation in an HDM model of asthma in female and male mice, as discussed in further detail in Example 3, below.

FIG. 24A-M illustrate that AIPB reduces retinal neurodegeneration in D2 glaucomatous mice, as discussed in further detail in Example 4, below.

FIG. 25A-D illustrates the AIPB reduces retinal neurodegeneration and improves visual function in a microbead-induced hypertension mouse model, as discussed in further detail in Example 4, below.

FIG. 26A-B illustrate that AIPB reduces retinal neurodegeneration in a mouse nerve crash model, as discussed in further detail in Example 4, below.

Like reference symbols in the various drawings indicate like elements.

Reference will now be made in detail to various exemplary embodiments provided herein, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, provided are compositions and methods using pharmaceutical compounds and formulations comprising nucleic acids, polypeptides, and gene and polypeptide delivery vehicles for regulating or manipulating, including modification of amino acid sequence, adding, maintaining, enhancing or upregulating, the expression of recombinant ApoA-I Binding Protein (APOA1BP, AIBP, or AI-BP), and kits comprising all or some of the components for practicing these compositions and methods. In alternative embodiments, provided are compositions and methods for altering AIBP sequence and structure and delivering therapeutic levels of recombinant AIBP to the body, including the brain and CNS, including use of delivery vehicles targeting and/or capable of penetrating the blood brain barrier, and nucleic acid (gene) delivery vehicles such as vectors and viruses such as an adeno-associated virus (AAV) delivery vehicle having contained within an AIBP expressing nucleic acid; and for direct delivery of either AIBP polypeptide or AIBP-expressing nucleic acid directly via intrathecal (i.t.) administration.

Example 1 describes studies using a mouse model of chemotherapy-induced peripheral neuropathy, where spinal microglia are characterized by the presence of inflammarafts—enlarged, cholesterol-enriched lipid rafts, which organize the inflammatory response. Manipulation of specific mechanisms regulated cholesterol metabolism and normalized inflammarafts and reprogramed microglia, resulting in a long-lasting alleviation of neuropathic pain.

We also show that a deletion mutant of AIBP that lacks the TLR4-binding domain does not reverse neuropathic pain in a mouse model of chemotherapy-induced peripheral neuropathy. AIBP binding to TLR4 is important because this innate immune receptor is highly expressed in inflammatory cells and concentrates in lipid rafts on the cell surface and mediates inflammatory responses. Enlarged/clustered lipid rafts with increased content of TLR4 and the evidence of TLR4 dimerization are called “inflammarafts”. By virtue of binding to TLR4, AIBP targets inflammatory cells, disrupts inflammarafts and inhibits inflammation—spinal neuroinflammation and neuropathic pain, and the effect is applicable to many inflammatory disease states mediated by TLR4.

We also found that in native AIBP the N-terminal TLR4 binding domain is cryptic and the native AIBP does not bind to TLR4. The TLR4 binding domain in AIBP becomes exposed when the N-terminus is extended with additional amino acids, for example, as in the recombinantly engineered forms of AIBP as provided herein, as illustrated in FIG. 13. FIG. 11 is a graphical representation of this model.

In alternative embodiments, provided is an engineered AIBP comprising an amino acid sequence from the commercial pAcHLT-C vector (BD Biosciences).

TLR4 receptors localize to and dimerize in membrane lipid rafts. The enlarged, cholesterol-rich lipid rafts, harboring activated receptors and adaptor molecules—here designated as inflammarafts (Miller et al., 2020)—serve as an organizing platform to initiate inflammatory signaling and the cellular response. Regulation of cholesterol content in the plasma membrane can affect inflammarafts and TLR4 dimerization, signaling and inflammatory response in various cell types (Karasinska et al., 2013; Tall and Yvan-Charvet, 2015; Yvan-Charvet et al., 2008). In addition to TLR4, inflammarafts regulate activation of numerous other receptors and components of signaling pathways, as reviewed in (Miller et al., 2020). Thus, we hypothesized that CIPN was associated with altered cholesterol dynamics in spinal microglia, leading to inflammaraft formation and persistent neuroinflammation in the spinal cord.

To test this hypothesis, we measured spinal microglia lipid rafts and TLR4 dimerization in CIPN mice. To manipulate cholesterol dynamics, we used intrathecal injections of the apoA-I binding protein (AIBP), an effective multiplier of cholesterol removal from several cell types (Choi et al., 2018; Fang et al., 2013; Woller et al., 2018), and the mice with inducible, microglia-specific knockdown of the cholesterol transporters Abca1 and Abcg1. We demonstrate that AIBP induces redistribution of cholesterol in the microglia membrane, enhancing colocalization of accessible cholesterol with the cholesterol transporter ABCA1. This redistribution sets conditions for cholesterol depletion from the plasma membrane and the reversal of inflammarafts back to physiological lipid rafts. Microglia-specific Abca1/Abcg1 knockdown induces pain in naïve mice and prevents AIBP from reversing CIPN allodynia, highlighting the importance of microglial cholesterol homeostasis in the development of neuropathic pain. Furthermore, characterization of CIPN-associated changes in gene expression in microglia suggests impaired cholesterol metabolism.

### Recombinant AIBP Sequences

In alternative embodiments, engineered protein sequences are disclosed comprised of a ApoA-I Binding Protein (AIBP) amino acid sequence and an amino acid sequence N-terminal to the AIBP amino acid sequence, wherein the amino acid sequence N-terminal to the AIBP amino acid sequence comprises a peptide tag, wherein the peptide tag comprises a multi-histidine (multi-his) tag, in particular, the multi-his tag comprises six contiguous histidine residues (HHHHHH (SEQ ID NO:1)).

In other embodiments, the heterologous amino terminus amino acid sequence comprises the amino acid sequence MSPIDPMGHHHHHHGRRRASVAAGILVPRGSPGLDGICSR (SEQ ID NO:2) having mutation of its thrombin cleavage site so as to render it inoperable.

In some embodiments, provided is a peptide having an amino acid sequence produced from the commercial pAcHLT-C vector (BD Biosciences), wherein the amino acid sequence is comprised of a ApoA-I Binding Protein (AIBP) amino acid sequence and an amino acid sequence N-terminal to the AIBP amino acid sequence, wherein the amino acid sequence N-terminal to the AIBP amino acid sequence comprises a peptide tag, wherein the peptide tag comprises a multi-histidine (multi-his) tag.

In alternative embodiments, provided are methods for administering in vivo a recombinant or synthetic ApoA-I Binding Protein (APOA1BP, AIBP, or AI-BP) polypeptide compound or composition having a heterologous amino terminus amino acid sequence of at least about ten amino acid, or between about 10 to 100 amino acids, or between about 20 to 80 amino acids, or between about 30 to 50 amino acids, or any heterologous amino acid sequence sufficient to result in the unfolding and exposing of the cryptic (or hidden, unexposed) N-terminal TLR4 binding domain of the AIBP polypeptide.

In alternative embodiments, murine AIBP is used, for example, a murine AIBP having a sequence encoded by SEQ ID NO:3, and/or an amino acid sequence of SEQ ID NO:4, which optionally can be supplemented with (i.e., further comprise) a fibronectin secretion signal (italic) at the N-terminus, and/or with the His tag (underlined) at the C-terminus; the product is abbreviated as FIB-mAIBP-His:

SEQ ID NO: 3:
ATG CTC AGG GGT CCG GGA CCC GGG CGG CTG CTG CTG
CTA GCA GTC CTG TGC CTG GGG ACA TCG GTG CGC TGC
ACC GAA ACC GGG AAG AGC AAG AGG CAGCAGAGTGTGTGT
CGTGCAAGGCCCATCTGGTGGGGAACACAGCGCCGGGGCTCGGAGAC
CATGGCGGGCGCTGCGGTGAAGTACTTAAGTCAGGAGGAGGCTCAGG
CCGTGGACCAAGAGCTTTTTAACGAGTATCAGTTCAGCGTGGATCAA
CTCATGGAGCTGGCCGGGTTGAGCTGTGCCACGGCTATTGCCAAGGC
TTATCCCCCCACGTCTATGTCCAAGAGTCCCCCGACTGTCTTGGTCA
TCTGTGGCCCCGGAAATAACGGAGGGGATGGGCTGGTCTGTGCGCGA
CACCTCAAACTTTTTGGTTACCAGCCAACTATCTATTACCCCAAAAG
ACCTAACAAGCCCCTCTTCACTGGGCTAGTGACTCAGTGTCAGAAAA
TGGACATTCCTTTCCTTGGTGAAATGCCCCCAGAGCCCATGATGGTG
GACGAGCTGTATGAGCTGGTGGTGGACGCCATCTTCGGCTTCAGTTT
CAAGGGTGACGTTCGGGAGCCATTCCACAGCATCCTGAGTGTCTTGA
GTGGACTCACTGTGCCCATTGCTAGCATCGACATTCCCTCAGGATGG
GATGTAGAGAAGGGAAACCCTAGCGGAATCCAACCAGACTTACTCAT
CTCACTGACGGCACCCAAGAAGTCTGCAACTCACTTTACTGGCCGAT
ATCATTACCTTGGGGGTCGCTTTGTACCACCTGCTCTAGAGAAGAAG
TACCAGCTGAACCTGCCATCTTACCCTGACACAGAGTGTGTCTACCG
TCTACAGCATCATCATCATCATCATTAA
SEQ ID NO: 4:
MLRGPGPGRLLLLAVLCLGTSVRCTETGKSKRQQSVCRARPIWWGTQ
RRGSETMAGAAVKYLSQEEAQAVDQELFNEYQFSVDQLMELAGLSCA
TAIAKAYPPTSMSKSPPTVLVICGPGNNGGDGLVCARHLKLFGYQPT
IYYPKRPNKPLFTGLVTQCQKMDIPFLGEMPPEPMMVDELYELVVDA
IFGFSFKGDVREPFHSILSVLSGLTVPIASIDIPSGWDVEKGNPSGI
QPDLLISLTAPKKSATHFTGRYHYLGGRFVPPALEKKYQLNLPSYPD
TECVYRLQHHHHHH

In alternative embodiment, a variant of human AIBP (hAIBP) polypeptide as provided herein (for example, a human AIBP having heterologous amino acid sequence that results in exposure of a TLR4 (otherwise cryptic) binding site), or a nucleic acid encoding a variant AIBP as provided herein, is administered to a patient or an individual in need thereof, or is used to manufacture a formulation or pharmaceutical, or is used to make a vector or expression vehicle for administration, or is included in a kit as provided herein, and the AIBP variant can comprise or be encoded by:

Human AIBP-encoding nucleic acid (cDNA)
(SEQ ID NO: 5)
GGGCCGGGCCGGGCCGGGGGCGCGCGCTCTGCGAGCTGGATGTCCAG
GCTGCGGGCGCTGCTGGGCCTCGGGCTGCTGGTTGCGGGCTCGCGCG
TGCCGCGGATCAAAAGCCAGACCATCGCCTGTCGCTCGGGACCCACC
TGGTGGGGACCGCAGCGGCTGAACTCGGGTGGCCGCTGGGACTCAGA
GGTCATGGCGAGCACGGTGGTGAAGTACCTGAGCCAGGAGGAGGCCC
AGGCCGTGGACCAGGAGCTATTTAACGAATACCAGTTCAGCGTGGAC
CAACTTATGGAACTGGCCGGGCTGAGCTGTGCTACAGCCATCGCCAA
GGCATATCCCCCCACGTCCATGTCCAGGAGCCCCCCTACTGTCCTGG
TCATCTGTGGCCCGGGGAATAATGGAGGAGATGGTCTGGTCTGTGCT
CGACACCTCAAACTCTTTGGCTACGAGCCAACCATCTATTACCCCAA
AAGGCCTAACAAGCCCCTCTTCACTGCATTGGTGACCCAGTGTCAGA
AAATGGACATCCCTTTCCTTGGGGAAATGCCCGCAGAGCCCATGACG
ATTGATGAACTGTATGAGCTGGTGGTGGATGCCATCTTTGGCTTCAG
CTTCAAGGGCGATGTTCGGGAACCGTTCCACAGCATCCTGAGTGTCC
TGAAGGGACTCACTGTGCCCATTGCCAGCATCGACATTCCCTCAGGA
TGGGACGTGGAGAAGGGAAATGCTGGAGGGATCCAGCCAGACTTGCT
CATATCCCTCACAGCCCCCAAAAAATCTGCAACCCAGTTTACCGGTC
GCTACCATTACCTGGGGGGTCGTTTTGTGCCACCTGCTCTGGAGAAG
AAGTACCAGCTGAACCTGCCACCCTACCCTGACACCGAGTGTGTCTA
TCGTCTGCAGTGAGGGAAGGTGGGTGGGTATTCTTCCCAATAAAGAC
TTAGAGCCCCTCTCTTCCAGAACTGTGGATTCCTGGGAGCTCCTCTG
GCAATAAAAGTCAGTGAATGGTGGAAGTCAGAGACCAACCCTGGGGA
TTGGGTGCCATCTCTCTAGGGGTAACACAAAGGGCAAGAGGTTGCTA
TGGTATTTGGAAACAATGAAAATGGACTGTTAGATGCCAA
Human AIBP polypeptide
(SEQ ID NO: 6)
MSRLRALLGLGLLVAGSRVPRIKSQTIACRSGPTWWGPQRLNSGGRW
DSEVMASTVVKYLSQEEAQAVDQELFNEYQFSVDQLMELAGLSCATA
IAKAYPPTSMSRSPPTVLVICGPGNNGGDGLVCARHLKLFGYEPTIY
YPKRPNKPLFTALVTQCQKMDIPFLGEMPAEPMTIDELYELVVDAIF
GFSFKGDVREPFHSILSVLKGLTVPIASIDIPSGWDVEKGNAGGIQP
DLLISLTAPKKSATQFTGRYHYLGGRFVPPALEKKYQLNLPPYPDTE
CVYRLQ

In some embodiments, a modified hAIBP is used, that retains the TLR4-binding domain and has N-terminal residues replaced with a native signal peptide, for example, the hAIBP comprises amino acids 25-288 of the hAIBP sequence, also known as d24hAIBP (encoding nucleic acid):

(SEQ ID NO: 20)
CAGACCATCGCCTGTCGCTCGGGACCCACCTGGTGGGGACCGCAGCG
GCTGAACTCGGGTGGCCGCTGGGACTCAGAGGTCATGGCGAGCACGG
TGGTGAAGTACCTGAGCCAGGAGGAGGCCCAGGCCGTGGACCAGGAG
CTATTTAACGAATACCAGTTCAGCGTGGACCAACTTATGGAACTGGC
CGGGCTGAGCTGTGCTACAGCCATCGCCAAGGCATATCCCCCCACGT
CCATGTCCAGGAGCCCCCCTACTGTCCTGGTCATCTGTGGCCCGGGG
AATAATGGAGGAGATGGTCTGGTCTGTGCTCGACACCTCAAACTCTT
TGGCTACGAGCCAACCATCTATTACCCCAAAAGGCCTAACAAGCCCC
TCTTCACTGCATTGGTGACCCAGTGTCAGAAAATGGACATCCCTTTC
CTTGGGGAAATGCCCGCAGAGCCCATGACGATTGATGAACTGTATGA
GCTGGTGGTGGATGCCATCTTTGGCTTCAGCTTCAAGGGCGATGTTC
GGGAACCGTTCCACAGCATCCTGAGTGTCCTGAAGGGACTCACTGTG
CCCATTGCCAGCATCGACATTCCCTCAGGATGGGACGTGGAGAAGGG
AAATGCTGGAGGGATCCAGCCAGACTTGCTCATATCCCTCACAGCCC
CCAAAAAATCTGCAACCCAGTTTACCGGTCGCTACCATTACCTGGGG
GGTCGTTTTGTGCCACCTGCTCTGGAGAAGAAGTACCAGCTGAACCT
GCCACCCTACCCTGACACCGAGTGTGTCTATCGTCTGCAG

wherein the corresponding d24hAIBP polypeptide is:

(SEQ ID NO: 21)
QTIACRSGPTWWGPQRLNSGGRWDSEVMASTVVKYLSQEEAQAVDQE
LFNEYQFSVDQLMELAGLSCATAIAKAYPPTSMSRSPPTVLVICGPG
NNGGDGLVCARHLKLFGYEPTIYYPKRPNKPLFTALVTQCQKMDIPF
LGEMPAEPMTIDELYELVVDAIFGFSFKGDVREPFHSILSVLKGLTV
PIASIDIPSGWDVEKGNAGGIQPDLLISLTAPKKSATQFTGRYHYLG
GRFVPPALEKKYQLNLPPYPDTECVYRLQ.

In some embodiments, provided is a human AIBP in which a portion of the N-terminus of AIBP (amino acids 1-24, d24hAIBP) is replaced with (or further comprises) a fibronectin secretion signal (italic); the product is abbreviated as FIB-d24hAIBP and named Compound 1:

Human_FIB-d24hAIBP (Compound 1)-encoding
nucleic acid (cDNA):
(SEQ ID NO: 22)
ATGCTCAGGGGTCCGGGACCCGGGCGGCTGCTGCTGCTAGCAGTCCT
GTGCCTGGGGACATCGGTGCGCTGCACCGAAACCGGGAAGAGCAAGA
GGCAGACCATCGCCTGTCGCTCGGGACCCACCTGGTGGGGACCGCAG
CGGCTGAACTCGGGTGGCCGCTGGGACTCAGAGGTCATGGCGAGCAC
GGTGGTGAAGTACCTGAGCCAGGAGGAGGCCCAGGCCGTGGACCAGG
AGCTATTTAACGAATACCAGTTCAGCGTGGACCAACTTATGGAACTG
GCCGGGCTGAGCTGTGCTACAGCCATCGCCAAGGCATATCCCCCCAC
GTCCATGTCCAGGAGCCCCCCTACTGTCCTGGTCATCTGTGGCCCGG
GGAATAATGGAGGAGATGGTCTGGTCTGTGCTCGACACCTCAAACTC
TTTGGCTACGAGCCAACCATCTATTACCCCAAAAGGCCTAACAAGCC
CCTCTTCACTGCATTGGTGACCCAGTGTCAGAAAATGGACATCCCTT
TCCTTGGGGAAATGCCCGCAGAGCCCATGACGATTGATGAACTGTAT
GAGCTGGTGGTGGATGCCATCTTTGGCTTCAGCTTCAAGGGCGATGT
TCGGGAACCGTTCCACAGCATCCTGAGTGTCCTGAAGGGACTCACTG
TGCCCATTGCCAGCATCGACATTCCCTCAGGATGGGACGTGGAGAAG
GGAAATGCTGGAGGGATCCAGCCAGACTTGCTCATATCCCTCACAGC
CCCCAAAAAATCTGCAACCCAGTTTACCGGTCGCTACCATTACCTGG
GGGGTCGTTTTGTGCCACCTGCTCTGGAGAAGAAGTACCAGCTGAAC
CTGCCACCCTACCCTGACACCGAGTGTGTCTATCGTCTGCAG
(SEQ ID NO: 23)
MLRGPGPGRLLLLAVLCLGTSVRCTETGKSKRQTIACRSGPTWWGPQ
RLNSGGRWDSEVMASTVVKYLSQEEAQAVDQELFNEYQFSVDQLMEL
AGLSCATAIAKAYPPTSMSRSPPTVLVICGPGNNGGDGLVCARHLKL
FGYEPTIYYPKRPNKPLFTALVTQCQKMDIPFLGEMPAEPMTIDELY
ELVVDAIFGFSFKGDVREPFHSILSVLKGLTVPIASIDIPSGWDVEK
GNAGGIQPDLLISLTAPKKSATQFTGRYHYLGGRFVPPALEKKYQLN
LPPYPDTECVYRLQ

In this embodiment, the hAIBP fragment comprises amino acids 25 to 288 (also known as d24hAIBP) and the N-terminal modification is:

(SEQ ID NO: 24)
MLRGPGPGRLLLLAVLCLGTSVRCTETGKSKR.

In one embodiment, a secretion signal is added to ensure robust secretion of AIBP, for example, a fibronectin secretion signal is added to N terminus of AIBP (see italicized sequences in SEQ ID NO:3 and SEQ ID NO:4); or a nucleic acid encoding a secretion signal is added to the AIBP coding sequence. In alternative embodiments, a secretion signal is a fibronectin secretion signal, an immunoglobulin heavy chain secretion signal or an immunoglobulin kappa light chain secretory peptide (see, for example, PLOS One. 2015; 10(2): e0116878), or an interleukin-2 signal peptide (see, for example, J. Gene Med. 2005 March; 7(3):354-65).

In alternative embodiments, the polypeptide coding sequences are operatively linked to a promoter, for example, a constitutive, inducible, tissue specific (for example, nerve or brain tissue specific) or ubiquitous promoter or other transcriptional activating agent.

In other embodiments, the product from post-translational modification of the fibronectin-hAIBP construct has an amino acid sequence (Compound 2):

(SEQ ID NO: 25)
TETGKSKRQTIACRSGPTWWGPQRLNSGGRWDSEVMASTVVKYLSQE
EAQAVDQELFNEYQFSVDQLMELAGLSCATAIAKAYPPTSMSRSPPT
VLVICGPGNNGGDGLVCARHLKLFGYEPTIYYPKRPNKPLFTALVTQ
CQKMDIPFLGEMPAEPMTIDELYELVVDAIFGFSFKGDVREPFHSIL
SVLKGLTVPIASIDIPSGWDVEKGNAGGIQPDLLISLTAPKKSATQF
TGRYHYLGGRFVPPALEKKYQLNLPPYPDTECVYRLQ,

wherein the hAIBP fragment is d24hAIBP and the N-terminal modification is TETGKSKR (SEQ ID NO:26),

In other embodiments, the sequence of the AIBP polypeptide is modified at its C-terminus to incorporate additional peptidic fragments. This is exemplified by addition of a C-terminal His Tag (underlined in the corresponding amino acid sequence): (Compound 3 encoding nucleic acid sequence):

(SEQ ID NO: 27)
ATGCTCAGGGGTCCGGGACCCGGGCGGCTGCTGCTGCTAGCAGTCCT
GTGCCTGGGGACATCGGTGCGCTGCACCGAAACCGGGAAGAGCAAGA
GGCAGACCATCGCCTGTCGCTCGGGACCCACCTGGTGGGGACCGCAG
CGGCTGAACTCGGGTGGCCGCTGGGACTCAGAGGTCATGGCGAGCAC
GGTGGTGAAGTACCTGAGCCAGGAGGAGGCCCAGGCCGTGGACCAGG
AGCTATTTAACGAATACCAGTTCAGCGTGGACCAACTTATGGAACTG
GCCGGGCTGAGCTGTGCTACAGCCATCGCCAAGGCATATCCCCCCAC
GTCCATGTCCAGGAGCCCCCCTACTGTCCTGGTCATCTGTGGCCCGG
GGAATAATGGAGGAGATGGTCTGGTCTGTGCTCGACACCTCAAACTC
TTTGGCTACGAGCCAACCATCTATTACCCCAAAAGGCCTAACAAGCC
CCTCTTCACTGCATTGGTGACCCAGTGTCAGAAAATGGACATCCCTT
TCCTTGGGGAAATGCCCGCAGAGCCCATGACGATTGATGAACTGTAT
GAGCTGGTGGTGGATGCCATCTTTGGCTTCAGCTTCAAGGGCGATGT
TCGGGAACCGTTCCACAGCATCCTGAGTGTCCTGAAGGGACTCACTG
TGCCCATTGCCAGCATCGACATTCCCTCAGGATGGGACGTGGAGAAG
GGAAATGCTGGAGGGATCCAGCCAGACTTGCTCATATCCCTCACAGC
CCCCAAAAAATCTGCAACCCAGTTTACCGGTCGCTACCATTACCTGG
GGGGTCGTTTTGTGCCACCTGCTCTGGAGAAGAAGTACCAGCTGAAC
CTGCCACCCTACCCTGACACCGAGTGTGTCTATCGTCTGCAGTTGGT
CCCTCGTGGAAGCCATCATCATCATCATCA

### Amino Acid Sequence (Compound 3):

(SEQ ID NO: 28)
MLRGPGPGRLLLLAVLCLGTSVRCTETGKSKRQTIACRSGPTWWGP
QRLNSGGRWDSEVMASTVVKYLSQEEAQAVDQELFNEYQFSVDQLM
ELAGLSCATAIAKAYPPTSMSRSPPTVLVICGPGNNGGDGLVCARH
LKLFGYEPTIYYPKRPNKPLFTALVTQCQKMDIPFLGEMPAEPMTI
DELYELVVDAIFGFSFKGDVREPFHSILSVLKGLTVPIASIDIPSG
WDVEKGNAGGIQPDLLISLTAPKKSATQFTGRYHYLGGRFVPPALE
KKYQLNLPPYPDTECVYRLQLVPRGSHHHHH.

wherein the hAIBP fragment is d24hAIBP containing a His Tag (FIB-d24 AIBP-His) and the N-terminal modification:

(SEQ ID NO: 29)
LRGPGPGRLLLLAVLCLGTSVRCTETGKSKR,

In other embodiments the post-translational modification of the signal peptide provides a compound (Compound 4):

(SEQ ID NO: 30)
TETGKSKRQTIACRSGPTWWGPQRLNSGGRWDSEVMASTVVKYLSQE
EAQAVDQELFNEYQFSVDQLMELAGLSCATAIAKAYPPTSMSRSPPT
VLVICGPGNNGGDGLVCARHLKLFGYEPTIYYPKRPNKPLFTALVTQ
CQKMDIPFLGEMPAEPMTIDELYELVVDAIFGFSFKGDVREPFHSIL
SVLKGLTVPIASIDIPSGWDVEKGNAGGIQPDLLISLTAPKKSATQF
TGRYHYLGGRFVPPALEKKYQLNLPPYPDTECVYRLQLVPRGSHHHH
H,

wherein the hAIBP fragment is d24hAIBP-His and the N-terminal modification is TETGKSKR (SEQ ID NO:26),

In other embodiments, the polypeptide coding sequences are operatively linked to a promoter, e.g., a constitutive, inducible, tissue specific (e.g., nerve or brain tissue specific) or ubiquitous promoter or other transcriptional activating agent.

In other embodiments, full length human AIBP is modified at its N-terminus, wherein such modification facilitates TLR4 binding, for example Compound 5-encoding nucleic acid (cDNA):

(SEQ ID NO: 31)
ATGGACTACAAAGACCATGACGGTGATTATAAAGATCATGACATCGA
TTACAAGGATGACGATGACAAGCTTGCGGCCGCGAATTCAGGGCCGG
GGGCGCGCGCTCTGCGAGCTGGATGTCCAGGCTGCGGGCGCTGCTGG
GCCTCGGGCTGCTGGTTGCGGGCTCGCGCGTGCCGCGGATCAAAAGC
CAGACCATCGCCTGTCGCTCGGGACCCACCTGGTGGGGACCGCAGCG
GCTGAACTCGGGTGGCCGCTGGGACTCAGAGGTCATGGCGAGCACGG
TGGTGAAGTACCTGAGCCAGGAGGAGGCCCAGGCCGTGGACCAGGAG
CTATTTAACGAATACCAGTTCAGCGTGGACCAACTTATGGAACTGGC
CGGGCTGAGCTGTGCTACAGCCATCGCCAAGGCATATCCCCCCACGT
CCATGTCCAGGAGCCCCCCTACTGTCCTGGTCATCTGTGGCCCGGGG
AATAATGGAGGAGATGGTCTGGTCTGTGCTCGACACCTCAAACTCTT
TGGCTACGAGCCAACCATCTATTACCCCAAAAGGCCTAACAAGCCCC
TCTTCACTGCATTGGTGACCCAGTGTCAGAAAATGGACATCCCTTTC
CTTGGGGAAATGCCCGCAGAGCCCATGACGATTGATGAACTGTATGA
GCTGGTGGTGGATGCCATCTTTGGCTTCAGCTTCAAGGGCGATGTTC
GGGAACCGTTCCACAGCATCCTGAGTGTCCTGAAGGGACTCACTGTG
CCCATTGCCAGCATCGACATTCCCTCAGGATGGGACGTGGAGAAGGG
AAATGCTGGAGGGATCCAGCCAGACTTGCTCATATCCCTCACAGCCC
CCAAAAAATCTGCAACCCAGTTTACCGGTCGCTACCATTACCTGGGG
GGTCGTTTTGTGCCACCTGCTCTGGAGAAGAAGTACCAGCTGAACCT
GCCACCCTACCCTGACACCGAGTGTGTCTATCGTCTGCAGTGAGGGA
AGGTGGGGGGTATTCTTCCCAATAAAGACTTAGAGCCCCTCTCTTCC
AGAACTGTGGATTCCTGGGAGCTCCTCTGGCAATAAAAGTCAGTGAA
TGGTGGAAGTCAGAGACCAACCCTGGGGATTGGGTGCCATCTCTCTA
GGGGTAACACAAAGGGCAAGAGGTTGCTATGGTATTTGGAAACAATG
AAAATGGACTGTTAGATGCCAA,

This encodes the following amino acid (Compound 5):

(SEQ ID NO: 32)
MDYKDHDGDYKDHDIDYKDDDDKLAAANSMSRLRALLGLGLLVAGSR
VPRIKSQTIACRSGPTWWGPQRLNSGGRWDSEVMASTVVKYLSQEEA
QAVDQELFNEYQFSVDQLMELAGLSCATAIAKAYPPTSMSRSPPTVL
VICGPGNNGGDGLVCARHLKLFGYEPTIYYPKRPNKPLFTALVTQCQ
KMDIPFLGEMPAEPMTIDELYELVVDAIFGFSFKGDVREPFHSILSV
LKGLTVPIASIDIPSGWDVEKGNAGGIQPDLLISLTAPKKSATQFTG
RYHYLGGRFVPPALEKKYQLNLPPYPDTECVYRLQ,

wherein, the hAIBP fragment is full length and the N-terminal modification:

(SEQ ID NO: 33)
MDYKDHDGDYKDHDIDYKDDDDKLAAANS,

In other embodiments, hAIBP sequence which retains the cryptic TLR4 binding domain is modified at its N-terminus. Example sequences comprise DNA and peptide sequence for amino acids 25-288 of hAIBP (d24hAIBP):

### (Compound 6-Encoding Nucleic Acid Sequence):

(SEQ ID NO: 34)
ATGGACTACAAAGACCATGACGGTGATTATAAAGATCATGACATCG
ATTACAAGGATGACGATGACAAGCTTGCGGCCGCGAATTCACAGAC
CATCGCCTGTCGCTCGGGACCCACCTGGTGGGGACCGCAGCGGCTG
AACTCGGGTGGCCGCTGGGACTCAGAGGTCATGGCGAGCACGGTGG
TGAAGTACCTGAGCCAGGAGGAGGCCCAGGCCGTGGACCAGGAGCT
ATTTAACGAATACCAGTTCAGCGTGGACCAACTTATGGAACTGGCC
GGGCTGAGCTGTGCTACAGCCATCGCCAAGGCATATCCCCCCACGT
CCATGTCCAGGAGCCCCCCTACTGTCCTGGTCATCTGTGGCCCGGG
GAATAATGGAGGAGATGGTCTGGTCTGTGCTCGACACCTCAAACTC
TTTGGCTACGAGCCAACCATCTATTACCCCAAAAGGCCTAACAAGC
CCCTCTTCACTGCATTGGTGACCCAGTGTCAGAAAATGGACATCCC
TTTCCTTGGGGAAATGCCCGCAGAGCCCATGACGATTGATGAACTG
TATGAGCTGGTGGTGGATGCCATCTTTGGCTTCAGCTTCAAGGGCG
ATGTTCGGGAACCGTTCCACAGCATCCTGAGTGTCCTGAAGGGACT
CACTGTGCCCATTGCCAGCATCGACATTCCCTCAGGATGGGACGTG
GAGAAGGGAAATGCTGGAGGGATCCAGCCAGACTTGCTCATATCCC
TCACAGCCCCCAAAAAATCTGCAACCCAGTTTACCGGTCGCTACCA
TTACCTGGGGGGTCGTTTTGTGCCACCTGCTCTGGAGAAGAAGTAC
CAGCTGAACCTGCCACCCTACCCTGACACCGAGTGTGTCTATCGTC
TGCAG,

### Amino Acid Sequence (Compound 7):

(SEQ ID NO: 35)
MDYKDHDGDYKDHDIDYKDDDDKLAAANSQTIACRSGPTWWGPQRL
NSGGRWDSEVMASTVVKYLSQEEAQAVDQELFNEYQFSVDQLMELA
GLSCATAIAKAYPPTSMSRSPPTVLVICGPGNNGGDGLVCARHLKL
FGYEPTIYYPKRPNKPLFTALVTQCQKMDIPFLGEMPAEPMTIDEL
YELVVDAIFGFSFKGDVREPFHSILSVLKGLTVPIASIDIPSGWDV
EKGNAGGIQPDLLISLTAPKKSATQFTGRYHYLGGRFVPPALEKKY
QLNLPPYPDTECVYRLQ,

wherein, the hAIBP fragment is d24hAIBP and the N-terminal modification is:

(SEQ ID NO: 33)
MDYKDHDGDYKDHDIDYKDDDDKLAAANS,

### (Compound 7-Encoding Nucleic Acid Sequence):

(SEQ ID NO: 34)
ATG TCC CCT ATA GAT CCG ATG GGA CAT CAT CAT
CAT CAT CAC GGA AGG AGA AGG GCC AGT GTT GCG
GCG GGA ATT TTG GTC CCT CGT GGA AGC CCA GGA
CTC GAT GGC ATA TGC TCG AGGCAGACCATCGCCTGTCGCT
CGGGACCCACCTGGTGGGGACCGCAGCGGCTGAACTCGGGTGGCCG
CTGGGACTCAGAGGTCATGGCGAGCACGGTGGTGAAGTACCTGAGC
CAGGAGGAGGCCCAGGCCGTGGACCAGGAGCTATTTAACGAATACC
AGTTCAGCGTGGACCAACTTATGGAACTGGCCGGGCTGAGCTGTGC
TACAGCCATCGCCAAGGCATATCCCCCCACGTCCATGTCCAGGAGC
CCCCCTACTGTCCTGGTCATCTGTGGCCCGGGGAATAATGGAGGAG
ATGGTCTGGTCTGTGCTCGACACCTCAAACTCTTTGGCTACGAGCC
AACCATCTATTACCCCAAAAGGCCTAACAAGCCCCTCTTCACTGCA
TTGGTGACCCAGTGTCAGAAAATGGACATCCCTTTCCTTGGGGAAA
TGCCCGCAGAGCCCATGACGATTGATGAACTGTATGAGCTGGTGGT
GGATGCCATCTTTGGCTTCAGCTTCAAGGGCGATGTTCGGGAACCG
TTCCACAGCATCCTGAGTGTCCTGAAGGGACTCACTGTGCCCATTG
CCAGCATCGACATTCCCTCAGGATGGGACGTGGAGAAGGGAAATGC
TGGAGGGATCCAGCCAGACTTGCTCATATCCCTCACAGCCCCCAAA
AAATCTGCAACCCAGTTTACCGGTCGCTACCATTACCTGGGGGGTC
GTTTTGTGCCACCTGCTCTGGAGAAGAAGTACCAGCTGAACCTGCC
ACCCTACCCTGACACCGAGTGTGTCTATCGTCTGCAG,

wherein, the hAIBP fragment is d24hAIBP and the N-terminal modification is: MSPIDPMGHHHHHHGRRRASVAAGILVPRGSPGLDGICSR (SEQ ID NO:2),

### (Compound 8—Encoding Nucleic Acid Sequence):

(SEQ ID NO: 37)
ATG TCC CCT ATA GAT CCG ATG GGA CAT CAT CAT
CAT CAT CAC GGA AGG AGA AGG GCC AGT GTT GCG
GCG GGA ATT TTG GTC CCT CGT GGA AGC GAT GGA
GAC GAT GGC GAT GAC GAC AGG CAGACCATCGCCTGTCGC
TCGGGACCCACCTGGTGGGGACCGCAGCGGCTGAACTCGGGTGGCC
GCTGGGACTCAGAGGTCATGGCGAGCACGGTGGTGAAGTACCTGAG
CCAGGAGGAGGCCCAGGCCGTGGACCAGGAGCTATTTAACGAATAC
CAGTTCAGCGTGGACCAACTTATGGAACTGGCCGGGCTGAGCTGTG
CTACAGCCATCGCCAAGGCATATCCCCCCACGTCCATGTCCAGGAG
CCCCCCTACTGTCCTGGTCATCTGTGGCCCGGGGAATAATGGAGGA
GATGGTCTGGTCTGTGCTCGACACCTCAAACTCTTTGGCTACGAGC
CAACCATCTATTACCCCAAAAGGCCTAACAAGCCCCTCTTCACTGC
ATTGGTGACCCAGTGTCAGAAAATGGACATCCCTTTCCTTGGGGAA
ATGCCCGCAGAGCCCATGACGATTGATGAACTGTATGAGCTGGTGG
TGGATGCCATCTTTGGCTTCAGCTTCAAGGGCGATGTTCGGGAACC
GTTCCACAGCATCCTGAGTGTCCTGAAGGGACTCACTGTGCCCATT
GCCAGCATCGACATTCCCTCAGGATGGGACGTGGAGAAGGGAAATG
CTGGAGGGATCCAGCCAGACTTGCTCATATCCCTCACAGCCCCCAA
AAAATCTGCAACCCAGTTTACCGGTCGCTACCATTACCTGGGGGGT
CGTTTTGTGCCACCTGCTCTGGAGAAGAAGTACCAGCTGAACCTGC
CACCCTACCCTGACACCGAGTGTGTCTATCGTCTGCAG,

### (Compound 6):

(SEQ ID NO: 36)
MSPIDPMGHHHHHHGRRRASVAAGILVPRGSDGDDGDDDRQTIACR
SGPTWWGPQRLNSGGRWDSEVMASTVVKYLSQEEAQAVDQELFNEY
QFSVDQLMELAGLSCATAIAKAYPPTSMSRSPPTVLVICGPGNNGG
DGLVCARHLKLFGYEPTIYYPKRPNKPLFTALVTQCQKMDIPFLGE
MPAEPMTIDELYELVVDAIFGFSFKGDVREPFHSILSVLKGLTVPI
ASIDIPSGWDVEKGNAGGIQPDLLISLTAPKKSATQFTGRYHYLGG
RFVPPALEKKYQLNLPPYPDTECVYRLQ,

wherein, the hAIBP fragment is d24hAIBP and the N-terminal modification is:

(SEQ ID NO: 19)
MSPIDPMGHHHHHHGRRRASVAAGILVPRGSDGDDGDDDR,

### (Compound 7-Encoding Nucleic Acid Sequence):

(SEQ ID NO: 16)
ATG TCC CCT ATA GAT CCG ATG GGA CAT CAT CAT
CAT CAT CAC GGA AGG AGA AGG GCC AGT GTT GCG
GCG GGA ATT TTG GTC CCT CGT GGA AGC GAT GGA
GAC GAT GGC ATA TGC TCG AGG CAGACCATCGCCTGTCGC
TCGGGACCCACCTGGTGGGGACCGCAGCGGCTGAACTCGGGTGGCC
GCTGGGACTCAGAGGTCATGGCGAGCACGGTGGTGAAGTACCTGAG
CCAGGAGGAGGCCCAGGCCGTGGACCAGGAGCTATTTAACGAATAC
CAGTTCAGCGTGGACCAACTTATGGAACTGGCCGGGCTGAGCTGTG
CTACAGCCATCGCCAAGGCATATCCCCCCACGTCCATGTCCAGGAG
CCCCCCTACTGTCCTGGTCATCTGTGGCCCGGGGAATAATGGAGGA
GATGGTCTGGTCTGTGCTCGACACCTCAAACTCTTTGGCTACGAGC
CAACCATCTATTACCCCAAAAGGCCTAACAAGCCCCTCTTCACTGC
ATTGGTGACCCAGTGTCAGAAAATGGACATCCCTTTCCTTGGGGAA
ATGCCCGCAGAGCCCATGACGATTGATGAACTGTATGAGCTGGTGG
TGGATGCCATCTTTGGCTTCAGCTTCAAGGGCGATGTTCGGGAACC
GTTCCACAGCATCCTGAGTGTCCTGAAGGGACTCACTGTGCCCATT
GCCAGCATCGACATTCCCTCAGGATGGGACGTGGAGAAGGGAAATG
CTGGAGGGATCCAGCCAGACTTGCTCATATCCCTCACAGCCCCCAA
AAAATCTGCAACCCAGTTTACCGGTCGCTACCATTACCTGGGGGGT
CGTTTTGTGCCACCTGCTCTGGAGAAGAAGTACCAGCTGAACCTGC
CACCCTACCCTGACACCGAGTGTGTCTATCGTCTGCAG,

### Amino Acid Sequence (Compound 7):

(SEQ ID NO: 15)
MSPIDPMGHHHHHHGRRRASVAAGILVPRGSDGDDGICSRQTIACR
SGPTWWGPQRLNSGGRWDSEVMASTVVKYLSQEEAQAVDQELFNEY
QFSVDQLMELAGLSCATAIAKAYPPTSMSRSPPTVLVICGPGNNGG
DGLVCARHLKLFGYEPTIYYPKRPNKPLFTALVTQCQKMDIPFLGE
MPAEPMTIDELYELVVDAIFGFSFKGDVREPFHSILSVLKGLTVPI
ASIDIPSGWDVEKGNAGGIQPDLLISLTAPKKSATQFTGRYHYLGG
RFVPPALEKKYQLNLPPYPDTECVYRLQ,

wherein, the hAIBP fragment is d24hAIBP and the N-terminal modification is: MSPIDPMGHHHHHHGRRRASVAAGILVPRGSDGDDGDDDR (SEQ ID NO:19),

In other embodiments, provided are compositions for a recombinant, synthetic ApoA-I Binding Protein (APOA1BP, AIBP, or AI-BP) polypeptide compound or composition having a heterologous amino terminus amino acid sequence of at least about eight amino acid, or between about 8 to 100 amino acids, or between about 8 to 40 amino acids, or between about 30 to 50 amino acids, or any heterologous amino acid sequence sufficient to result in the unfolding and exposing of the cryptic (or hidden, unexposed) N-terminal TLR4 binding domain of the AIBP polypeptide.

In alternative embodiments, the amino acid N-terminal sequence comprises between 3 and 12 basic amino acids selected from histidine (H), lysine (K) or arginine (R).

In other embodiments, compounds described herein may be further modified to improve properties for bioactivity, for example by removal of putative peptide cleavage sites. Example sequences are represented by:

### (Compound 8 Encoding Nucleic Acid Sequence):

(SEQ ID NO: 12)
ATG TCC CCT ATA GAT CCG ATG GGA CAT CAT CAT
CAT CAT CAC GGA AGG AGA AGG GCC AGT GTT GCG
GCG GGA ATT TTG GTC CCT GCT GCA AGC CCA GGA
CTC GAT GGC ATA TGC TCG AGG CAGACCATCGCCTGTCGC
TCGGGACCCACCTGGTGGGGACCGCAGCGGCTGAACTCGGGTGGCC
GCTGGGACTCAGAGGTCATGGCGAGCACGGTGGTGAAGTACCTGAG
CCAGGAGGAGGCCCAGGCCGTGGACCAGGAGCTATTTAACGAATAC
CAGTTCAGCGTGGACCAACTTATGGAACTGGCCGGGCTGAGCTGTG
CTACAGCCATCGCCAAGGCATATCCCCCCACGTCCATGTCCAGGAG
CCCCCCTACTGTCCTGGTCATCTGTGGCCCGGGGAATAATGGAGGA
GATGGTCTGGTCTGTGCTCGACACCTCAAACTCTTTGGCTACGAGC
CAACCATCTATTACCCCAAAAGGCCTAACAAGCCCCTCTTCACTGC
ATTGGTGACCCAGTGTCAGAAAATGGACATCCCTTTCCTTGGGGAA
ATGCCCGCAGAGCCCATGACGATTGATGAACTGTATGAGCTGGTGG
TGGATGCCATCTTTGGCTTCAGCTTCAAGGGCGATGTTCGGGAACC
GTTCCACAGCATCCTGAGTGTCCTGAAGGGACTCACTGTGCCCATT
GCCAGCATCGACATTCCCTCAGGATGGGACGTGGAGAAGGGAAATG
CTGGAGGGATCCAGCCAGACTTGCTCATATCCCTCACAGCCCCCAA
AAAATCTGCAACCCAGTTTACCGGTCGCTACCATTACCTGGGGGGT
CGTTTTGTGCCACCTGCTCTGGAGAAGAAGTACCAGCTGAACCTGC
CACCCTACCCTGACACCGAGTGTGTCTATCGTCTGCAG,

### Amino Acid Sequence (Compound 8):

(SEQ ID NO: 8)
MSPIDPMGHHHHHHGRRRASVAAGILVPAASPGLDGICSRQTIACR
SGPTWWGPQRLNSGGRWDSEVMASTVVKYLSQEEAQAVDQELFNEY
QFSVDQLMELAGLSCATAIAKAYPPTSMSRSPPTVLVICGPGNNGG
DGLVCARHLKLFGYEPTIYYPKRPNKPLFTALVTQCQKMDIPFLGE
MPAEPMTIDELYELVVDAIFGFSFKGDVREPFHSILSVLKGLTVPI
ASIDIPSGWDVEKGNAGGIQPDLLISLTAPKKSATQFTGRYHYLGG
RFVPPALEKKYQLNLPPYPDTECVYRLQ,

wherein, the hAIBP fragment is d24hAIBP and the N-terminal modification is:

(SEQ ID NO: 7)
MSPIDPMGHHHHHHGRRRASVAAGILVPAASPGLDGICSR.

In this sequence the thrombin cleavage site LVPRGS (SEQ ID NO:13) incorporates described amino acid mutation and prevents cleavage and unexpected loss of TLR4 binding activity as described in Example 2.

It should be acknowledged that these sequences are illustrative and are not limiting to the invention.

In other embodiments, any of the amino acids in the N-terminal modification of hAIBP may be unnatural and inserted by methods known to those skilled in the art.

### Products of Manufacture, Kits

Also provided are products of manufacture such as implants or pumps, kits and pharmaceuticals for practicing the methods as provided herein. In alternative embodiments, provided are products of manufacture, kits and/or pharmaceuticals comprising all the components needed to practice a method as provided herein. In alternative embodiments, kits also comprise instructions for practicing a method as provided herein,

### Formulations and Pharmaceutical Compositions

In alternative embodiments, provided are pharmaceutical formulations or compositions comprising nucleic acids and polypeptides for practicing methods and uses as provided herein to regulate neuropathic pain, the methods comprising upregulating the expression of recombinant ApoA-I Binding Protein (APOA1BP, AIBP, or AI-BP). In alternative embodiments, provided are pharmaceutical formulations or compositions for use in in vivo, in vitro or ex vivo methods to treat, prevent, reverse and/or ameliorate neuropathic pain. In alternative embodiments, pharmaceutical compositions and formulations used to practice methods and uses as provided herein comprise recombinant APOA1BP nucleic acids and polypeptides or result in an increase in expression or activity of recombinant APOA1BP nucleic acids and polypeptides are administered to an individual in need thereof in an amount sufficient to treat, prevent, reverse and/or ameliorate, for example, a neuropathic pain, a neurodegenerative disease or condition, optionally a chronic or progressive neurodegenerative disease, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS), optionally glaucoma or other inflammatory diseases of the eye, optionally lung inflammation and asthma, optionally HIV infection or its comorbidities, and/or optionally vascular inflammation, atherosclerosis and cardiovascular disease. In alternative embodiments, pharmaceutical compositions and formulations used to practice methods and uses as provided herein comprise recombinant APOA1BP nucleic acids and polypeptides or result in an increase in expression or activity of APOA1BP nucleic acids and polypeptides are administered to an individual in need thereof in an amount sufficient to prevent or decrease the intensity of and/or frequency of for example, the neuropathic pain or neurodegenerative disease or condition.

In alternative embodiments, the pharmaceutical compositions used to practice methods and uses as provided herein can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, these compositions used to practice methods and uses as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods or uses as provided herein can be in forms such as tablets, pills, powders, capsules, liquids, gels, syrups, slurries, suspensions, etc., suitable for in vivo, in vitro or ex vivo applications.

In alternative embodiments, formulations and pharmaceutical compositions used to practice methods and uses as provided herein can comprise a solution of compositions (which include peptidomimetics, racemic mixtures or racemates, isomers, stereoisomers, derivatives and/or analogs of compounds) disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice methods and uses as provided herein are sterile and can be manufactured to be generally free of undesirable matter. In one embodiment, these solutions and formulations are sterilized by conventional, well known sterilization techniques.

The solutions and formulations used to practice methods and uses as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compositions and formulations used to practice methods and uses as provided herein can be delivered by the use of liposomes. By using liposomes, particularly where the liposome surface carries ligands specific for target cells (for example, an injured or diseased neuronal cell or CNS tissue), or are otherwise preferentially directed to a specific tissue or organ type, one can focus the delivery of the active agent into a target cells in an in vivo, in vitro or ex vivo application.

### Nanoparticles, Nanolipoparticles and Liposomes

Also provided are nanoparticles, nanolipoparticles, vesicles and liposomal membranes comprising compounds used to practice methods and uses as provided herein, for example, to deliver compositions comprising recombinant APOA1BP nucleic acids and polypeptides in vivo, for example, to the CNS and brain. In alternative embodiments, these compositions are designed to target specific molecules, including biologic molecules, such as polypeptides, including cell surface polypeptides, for example, for targeting a desired cell type or organ, for example, a nerve cell or the CNS, and the like.

Provided are multilayered liposomes comprising compounds used to practice methods and uses as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods and uses as provided herein.

Liposomes can be made using any method, for example, as described in Park, et al., U.S. Pat. Pub. No. 20070042031, including method of producing a liposome by encapsulating an active agent (for example, recombinant APOA1BP nucleic acids and polypeptides), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods and uses as provided herein comprise a substituted ammonium and/or polyanions, for example, for targeting delivery of a compound (for example, a recombinant APOA1BP nucleic acid and polypeptide) to a desired cell type (for example, an endothelial cell, a nerve cell, or any tissue or area, for example, a CNS, in need thereof), as described for example, in U.S. Pat. Pub. No. 20070110798.

Provided are nanoparticles comprising compounds (for example, recombinant APOA1BP nucleic acids and polypeptides used to practice methods provided herein) in the form of active agent-containing nanoparticles (for example, a secondary nanoparticle), as described, for example, in U.S. Pat. Pub. No. 20070077286. In one embodiment, provided are nanoparticles comprising a fat-soluble active agent or a fat-solubilized water-soluble active agent to act with a bivalent or trivalent metal salt.

In one embodiment, solid lipid suspensions can be used to formulate and to deliver compositions used to practice methods and uses as provided herein to mammalian cells in vivo, for example, to the CNS, as described, for example, in U.S. Pat. Pub. No. 20050136121. Delivery Vehicle Modifications and Modification of AIBP

In alternative embodiments, recombinant AIBP peptides or polypeptides, or AIBP-comprising nanoparticles, liposomes and the like (for example, comprising or having contained therein recombinant APOA1BP nucleic acids or polypeptides used to practice methods provided herein) are modified to facilitate intrathecal injection, for example, delivery into the cerebrospinal fluid or brain. For example, in alternative embodiments, AIBP peptides or polypeptides, or recombinant AIBP-comprising nanoparticles, liposomes and the like, are engineered to comprise a moiety that allows the AIBP peptides or polypeptides, or AIBP-comprising nanoparticles, liposomes and the like, to bind to a receptor or cell membrane structure that facilitates delivery into the CNS or brain, for example, where the moiety can comprise a mannose-6-phosphate receptor, a melanotransferrin receptor, a LRP receptor or any other receptor that is ubiquitously expressed on the surface of any CNS or brain cell. For example, conjugation of mannose-6-phosphate moieties allows the AIBP peptides or polypeptides, or recombinant AIBP-comprising nanoparticles, liposomes and the like, to be taken up by a CNS cell that expresses a mannose-6-phosphate receptor. In alternative embodiments, any protocol or modification of the AIBP peptides or polypeptides, or AIBP-comprising nanoparticles, liposomes and the like, that facilitates entry or delivery into the CNS or brain in vivo can be used, for example, as described in U.S. Pat. No. 9,089,566.

In alternative embodiments, recombinant AIBP peptides or polypeptides, or AIBP-comprising nanoparticles, liposomes and the like (for example, comprising or having contained therein recombinant APOA1BP nucleic acids or polypeptides used to practice methods provided herein) are directly or indirectly linked or conjugated to any blood brain barrier (BBB)-targeting agent, for example, a transferrin, an insulin, a leptin, an insulin-like growth factor, a cationic peptide, a lectin, a Receptor-Associated Protein (RAP) (a 39 kD chaperone localized to the endoplasmic reticulum and Golgi, a lipoprotein receptor-related protein (LRP) receptor family ligand), an apolipoprotein B-100 derived peptide, an antibody (for example, a peptidomimetic monoclonal antibody) to a transferrin receptor, an antibody (for example, a peptidomimetic monoclonal antibody) to the insulin receptor, an antibody (for example, a peptidomimetic monoclonal antibody) to the insulin-like growth factor receptor, an antibody (for example, a peptidomimetic monoclonal antibody) to the leptin receptor and the like. In alternative embodiments, any protocol or modification of the AIBP peptides or polypeptides, or AIBP-comprising nanoparticles, liposomes and the like, that facilitates crossing of the BBB can be used, for example, as described in US Pat App Pub nos. 20050142141; 20050042227. For example, to enhance CNS or brain delivery of an composition used to practice methods as provided herein, any protocol can be used, for example: direct intra-cranial injection, transient permeabilization of the BBB, and/or modification of AIBP peptides or polypeptides, or AIBP-comprising nanoparticles, liposomes and the like to alter tissue distribution

### Delivery Cells and Delivery Vehicles

In alternative embodiments, any delivery vehicle can be used to practice the methods or uses as provided herein, for example, to deliver compositions (for example, recombinant APOA1BP nucleic acids and polypeptides) to a CNS or a brain in vivo. For example, delivery vehicles comprising polycations, cationic polymers and/or cationic peptides, such as polyethyleneimine derivatives, can be used for example as described, for example, in U.S. Pat. Pub. No. 20060083737. In one embodiment, a delivery vehicle is a transduced cell engineered to express or overexpress and then secrete an endogenous or exogenous AIBP.

In one embodiment, a dried polypeptide-surfactant complex is used to formulate a composition used to practice methods as provided herein, for example as described, for example, in U.S. Pat. Pub. No. 20040151766.

In one embodiment, a composition used to practice methods and uses as provided herein can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, cells that will be subsequently delivered to a CNS or a brain are transfected or transduced with an AIBP-expressing nucleic acid, for example, a vector, for example, by electro-permeabilization, which can be used as a primary or adjunctive means to deliver the composition to a cell, for example, using any electroporation system as described for example in U.S. Pat. Nos. 7,109,034; 6,261,815; 5,874,268.

### In Vivo Delivery of AIBP-Encoding Nucleic

In alternative embodiments, provided are compositions and methods for delivering nucleic acids encoding AIBP peptides or polypeptides, or nucleic acids encoding peptides or polypeptides having AIBP activity, or vectors or recombinant viruses having contained therein these nucleic acids. In alternative embodiments, the nucleic acids, vectors or recombinant viruses are designed for in vivo or CNS delivery and expression.

In alternative embodiments, provided are compositions and methods for the delivery and controlled expression of a recombinant AIBP-encoding nucleic acid or gene, or an expression vehicle (for example, vector, recombinant virus, and the like) comprising (having contained therein) a recombinant AIBP encoding nucleic acid or gene, that results in an AIBP protein being released into the bloodstream or general circulation where it can have a beneficial effect on in the body, for example, such as the CNS, brain or other targets.

In alternative embodiments, the provided are methods for being able to turn on and turn off AIBP-expressing nucleic acid or gene expression easily and efficiently for tailored treatments and insurance of optimal safety.

In alternative embodiments, recombinant AIBP protein or proteins expressed by the AIBP-expressing nucleic acid(s) or gene(s) have a beneficial or favorable effects (for example, therapeutic or prophylactic) on a tissue or an organ, for example, the brain, CNS, or other targets, even though secreted into the blood or general circulation at a distance (for example, anatomically remote) from their site or sites of action.

In alternative embodiments, provided are expression vehicles, vectors, recombinant viruses and the like for in vivo expression of a recombinant AIBP-encoding nucleic acid or gene to practice the methods as provide herein. In alternative embodiments, the expression vehicles, vectors, recombinant viruses and the like expressing the an AIBP nucleic acid or gene can be delivered by intramuscular (IM) injection, by intravenous (IV) injection, by subcutaneous injection, by inhalation, by a biolistic particle delivery system (for example, a so-called “gene gun”), and the like, for example, as an outpatient, for example, during an office visit.

In alternative embodiments, this “peripheral” mode of delivery, for example, expression vehicles, vectors, recombinant viruses and the like injected IM or IV, can circumvent problems encountered when genes or nucleic acids are expressed directly in an organ (for example, the brain or CNS) itself. Sustained secretion of an AIBP in the bloodstream or general circulation also circumvents the difficulties and expense of administering proteins by infusion.

In alternative embodiments a recombinant virus (for example, a long-term virus or viral vector), or a vector, or an expression vector, and the like, can be injected, for example, in a systemic vein (for example, IV), or by intramuscular (IM) injection, by inhalation, or by a biolistic particle delivery system (for example, a so-called “gene gun”), for example, as an outpatient, for example, in a physician's office. In alternative embodiments, days or weeks later (for example, four weeks later), the individual, patient or subject is administered (for example, inhales, is injected or swallows), a chemical or pharmaceutical that induces expression of the AIBP-expressing nucleic acids or genes; for example, an oral antibiotic (for example, doxycycline or rapamycin) is administered once daily (or more or less often), which will activate the expression of the gene. In alternative embodiments, after the “activation”, or inducement of expression (for example, by an inducible promoter) of the nucleic acid or gene, an AIBP protein is synthesized and released into the subject's circulation (for example, into the blood), and subsequently has favorable physiological effects, for example, therapeutic or prophylactic, that benefit the individual or patient (for example, benefit heart, kidney or lung function). When the physician or subject desires discontinuation of the AIBP treatment, the subject simply stops taking the activating chemical or pharmaceutical, for example, antibiotic. Alternative embodiments comprise use of “expression cassettes” comprising or having contained therein a nucleotide sequence used to practice methods provided herein, for example, an AIBP-expressing nucleic acid, which can be capable of affecting expression of the nucleic acid, for example, as a structural gene or a transcript (for example, encoding an AIBP protein) in a host compatible with such sequences. Expression cassettes can include at least a promoter operably linked with the polypeptide coding sequence or inhibitory sequence; and, in one aspect, with other sequences, for example, transcription termination signals. Additional factors necessary or helpful in effecting expression may also be used, for example, enhancers.

In alternative aspects, expression cassettes also include plasmids, expression vectors, recombinant viruses, any form of recombinant “naked DNA” vector, and the like. In alternative aspects, a “vector” can comprise a nucleic acid that can infect, transfect, transiently or permanently transduce a cell. In alternative aspects, a vector can be a naked nucleic acid, or a nucleic acid complexed with protein or lipid. In alternative aspects, vectors can comprise viral or bacterial nucleic acids and/or proteins, and/or membranes (for example, a cell membrane, a viral lipid envelope, etc.). In alternative aspects, vectors can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, a vector can be stably replicated by the cells during mitosis as an autonomous structure, or can be incorporated within the host's genome.

In alternative aspects, “promoters” include all sequences capable of driving transcription of a coding sequence in a cell, for example, a mammalian cell such as a muscle, nerve or brain cell. Promoters used in the constructs provided herein include cis-acting transcriptional control elements and regulatory sequences that are involved in regulating or modulating the timing and/or rate of transcription of a nucleic acid, for example, an AIBP-encoding nucleic acid. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

In alternative embodiments, “constitutive” promoters can be those that drive expression continuously under most environmental conditions and states of development or cell differentiation. In alternative embodiments, “inducible” or “regulatable” promoters can direct expression of a nucleic acid, for example, an AIBP-encoding nucleic acid, under the influence of environmental conditions, administered chemical agents, or developmental conditions.

### Gene Therapy and Gene Delivery Vehicles

In alternative embodiments, methods of the invention comprise use of nucleic acid (for example, gene or polypeptide encoding a recombinant AIBP-encoding nucleic acid) delivery systems to deliver a payload of the nucleic acid or gene, or AIBP-expressing nucleic acid, transcript or message, to a cell or cells in vitro, ex vivo, or in vivo, for example, as gene therapy delivery vehicles.

In alternative embodiments, expression vehicle, vector, recombinant virus, or equivalents used to practice methods provided herein are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh.10hCLN2; an organ-tropic AAV, or a neurotropic AAV; and/or an AAV capsid mutant or AAV hybrid serotype. In alternative embodiments, the AAV is engineered to increase efficiency in targeting a specific cell type that is non-permissive to a wild type (wt) AAV and/or to improve efficacy in infecting only a cell type of interest. In alternative embodiments, the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid. It is well known in the art how to engineer an adeno-associated virus (AAV) capsid in order to increase efficiency in targeting specific cell types that are non-permissive to wild type (wt) viruses and to improve efficacy in infecting only the cell type of interest; see for example, Wu et al., Mol. Ther. 2006 September; 14(3):316-27. Epub 2006 Jul. 7; Choi, et al., Curr. Gene Ther. 2005 June; 5(3):299-310.

For example, in alternative embodiments, serotypes AAV-8, AAV-9, AAV-DJ or AAV-DJ/8™ (Cell Biolabs, Inc., San Diego, CA), which have increased uptake in brain tissue in vivo, are used to deliver an AIBP-encoding nucleic acid payload for expression in the CNS. In alternative embodiments, the following serotypes, or variants thereof, are used for targeting a specific tissue:

| Tissue | Optimal Serotype |
|---|---|
| CNS | AAV1, AAV2, AAV4, AAV5, AAV8, AAV9 |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal | AAV1, AAV2, AAV4, AAV5, AAV8 |
| Pigment Epithelium) | |
| Skeletal Muscle | AAV1, AAV6, AAV7, AAV8, AAV9 |

In alternative embodiments, the rhesus-derived AAV AAVrh. 10hCLN2 or equivalents thereof can be used, wherein the rhesus-derived AAV may not be inhibited by any pre-existing immunity in a human; see for example, Sondhi, et al., Hum Gene Ther. Methods. 2012 October; 23(5):324-35, Epub 2012 Nov. 6; Sondhi, et al., Hum Gene Ther. Methods. 2012 Oct. 17; teaching that direct administration of AAVrh. 10hCLN2 to the CNS of rats and non-human primates at doses scalable to humans has an acceptable safety profile and mediates significant payload expression in the CNS.

Because adeno-associated viruses (AAVs) are common infective agents of primates, and as such, healthy primates carry a large pool of AAV-specific neutralizing antibodies (NAbs) which inhibit AAV-mediated gene transfer therapeutic strategies, methods provided herein can comprise screening of patient candidates for AAV-specific NAbs prior to treatment, especially with the frequently used AAV8 capsid component, to facilitate individualized treatment design and enhance therapeutic efficacy; see, for example, Sun, et al., J. Immunol. Methods. 2013 Jan. 31; 387(1-2):114-20, Epub 2012 Oct. 11.

### Dosaging

The pharmaceutical compositions and formulations used to practice methods and uses as provided herein can be administered for prophylactic and/or therapeutic treatments. In therapeutic applications, compositions are administered to a subject already suffering from a disease, condition, infection or defect in an amount sufficient to cure, alleviate or partially arrest the clinical manifestations of the disease, condition, infection or disease and its complications (a “therapeutically effective amount”), including for example, a neuropathic pain. For example, in alternative embodiments, recombinant APOA1BP nucleic acid- or polypeptide-comprising pharmaceutical compositions and formulations as provided herein are administered to an individual in need thereof in an amount sufficient to treat, prevent, reverse and/or ameliorate a neuropathic pain, an inflammation-induced neuropathic pain, an inflammation-induced neuropathic pain, a nerve or CNS inflammation, a allodynia, a post nerve injury pain or neuropathic pain, a post-surgical pain or neuropathic pain, a chemotherapeutic-induced peripheral neuropathy (CIPN) (for example, cisplatin-induced allodynia), a neurodegenerative disease or condition, optionally a chronic or progressive neurodegenerative disease or condition, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS), a migraine, a hyperalgesia, optionally glaucoma or other inflammatory diseases of the eye, optionally lung inflammation and asthma, optionally HIV infection or its comorbidities, and/or optionally vascular inflammation, atherosclerosis and cardiovascular disease.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In alternative embodiments, viral vectors such as adenovirus or AAV vectors are administered to an individual in need therein, and in alternative embodiment the dosage administered to a human comprises: a dose of about 2×10¹² vector genomes per kg body weight (vg/kg), or between about 10¹⁰ and 10¹⁴ vector genomes per kg body weight (vg/kg), or about 10⁹, 10¹⁰, 10¹¹, 10¹², 10¹³, 10¹⁴, 10¹⁵, or more vg/kg, which can be administered as a single dosage or in multiple dosages, as needed. In alternative embodiments, these dosages are administered orally, IM, IV, or intrathecally. In alternative embodiments, the vectors are delivered as formulations or pharmaceutical preparations, for example, where the vectors are contained in a nanoparticle, a particle, a micelle or a liposome or lipoplex, a polymersome, a polyplex or a dendrimer. In alternative embodiments, these dosages are administered once a day, once a week, or any variation thereof as needed to maintain in vivo expression levels of recombinant AIBP, which can be monitored by measuring actually expression of AIBP or by monitoring of therapeutic effect, for example, diminishing of pain. The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice methods as provided herein are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

### Bioisosteres of Compounds

In alternative embodiment, also provided are bioisosteres of compounds used to practice the methods provided herein, for example, polypeptides having a recombinant APOA1BP activity. Bioisosteres used to practice methods as provided herein include bioisosteres of, for example, recombinant APOA1BP nucleic acids and polypeptides, which in alternative embodiments can comprise one or more substituent and/or group replacements with a substituent and/or group having substantially similar physical or chemical properties which produce substantially similar biological properties to compounds used to practice methods or uses as provided herein. In one embodiment, the purpose of exchanging one bioisostere for another is to enhance the desired biological or physical properties of a compound without making significant changes in chemical structures.

For example, in one embodiment, one or more hydrogen atom(s) is replaced with one or more fluorine atom(s), for example, at a site of metabolic oxidation; this may prevent metabolism (catabolismfrom taking place. Because the fluorine atom is similar in size to the hydrogen atom the overall topology of the molecule is not significantly affected, leaving the desired biological activity unaffected. However, with a blocked pathway for metabolism, the molecule may have a longer half-life or be less toxic, and the like.

Devices for Delivering Therapeutic Agents Directly into the CNS or Brain

In alternative embodiments, pharmaceutical compositions and formulations, including nanoparticles and liposomes, used to practice methods as provided herein are delivered directly into a CNS or a brain, for example, either by injection intravenously or intrathecally, or by various devices known in the art. For example, U.S. Pat. App. Pub. No. 20080140056, describes a rostrally advancing catheter in the intrathecal space for direct brain delivery of pharmaceuticals and formulations. Implantable infusion devices can also be used; for example, a catheter to deliver fluid from the infusion device to the brain can be tunneled subcutaneously from the abdomen to the patient's skull, where the catheter can gain access to the individual's brain via a drilled hole. Alternatively, a catheter may be implanted such that it delivers the agent intrathecally within the patient's spinal canal. Flexible guide catheters having a distal end for introduction beneath the skull of a patient and a proximal end remaining external of the patient also can be used, for example, see U.S. Pat. App. Pub. No. 20060129126.

In alternative embodiments, pharmaceutical compositions and formulations used to practice methods as provided herein are delivered via direct delivery of pharmaceutical compositions and formulations, including nanoparticles and liposomes, or direct implantation of cells that express AIBP into a brain, for example, using any cell implantation cannula, syringe and the like, as described for example, in U.S. Pat. App. Pub. No. 20080132878; or elongate medical insertion devices as described for example, in U.S. Pat. No. 7,343,205; or a surgical cannula as described for example, in U.S. Pat. No. 4,899,729. Implantation cannulas, syringes and the like also can be used for direct injection of liquids, for example, as fluid suspensions.

In alternative embodiments, pharmaceutical compositions and formulations used to practice methods as provided herein are delivered with tracers that are detectable, for example, by magnetic resonance imaging (MRI) and/or by X-ray computed tomography (CT); the tracers can be co-infused with the therapeutic agent and used to monitor the distribution of the therapeutic agent as it moves through the target tissue, as described for example, in U.S. Pat. No. 7,371,225.

### Kits and Instructions

Provided are kits comprising compositions (including the devices as described herein) and/or instructions for practicing methods as provided herein to for example, treat, ameliorate or prevent a neuropathic pain. As such, kits, cells, vectors and the like can also be provided. In alternative embodiments, provided are kits comprising: a composition used to practice a method as provided herein, or a composition, a pharmaceutical composition or a formulation as provided herein, and optionally comprising instructions for use thereof.

The invention will be further described with reference to the examples described herein; however, it is to be understood that the invention is not limited to such examples.

### EXAMPLES

### Example 1: Efficacy Demonstrated in Exemplary Methods for Treating Pain

This example describes and demonstrates exemplary embodiments, and the efficacy of methods as provided herein to for example, treat or ameliorate a neuropathic pain, including for example, allodynia and TLR4-mediated inflammation-induced neuropathic pain.

Neuroinflammation is a major component in the transition to and perpetuation of neuropathic pain states. Spinal neuroinflammation involves activation of TLR4, localized to enlarged, cholesterol-enriched lipid rafts, designated here as inflammarafts. Conditional deletion of cholesterol transporters ABCA1 and ABCG1 in microglia, leading to inflammaraft formation, induced tactile allodynia in naïve mice. The apoA-I binding protein (AIBP) facilitated cholesterol depletion from inflammarafts and reversed neuropathic pain in a model of chemotherapy-induced peripheral neuropathy (CIPN) in wild-type mice, but AIBP (compound 7) failed to reverse allodynia in mice with ABCA1/ABCG1-deficient microglia, suggesting a cholesterol-dependent mechanism. An AIBP mutant lacking the TLR4-binding domain did not bind microglia nor reversed CIPN allodynia. The long-lasting therapeutic effect of a single AIBP (compound 7) dose in CIPN was associated with anti-inflammatory and cholesterol metabolism reprogramming and reduced accumulation of lipid droplets in microglia. These results suggest a cholesterol-driven mechanism of regulation of neuropathic pain by controlling TLR4 inflammarafts and gene expression program in microglia and blocking the perpetuation of neuroinflammation.

### Results

### Chemotherapy-Induced Peripheral Neuropathy Alters Lipid Rafts and TLR4 Dimerization in Spinal Microglia

In a model of chemotherapy-induced peripheral neuropathy (Woller et al., 2018), intraperitoneal injections of cisplatin induced severe tactile allodynia in male mice (FIG. 1A). This condition was associated with increased formation of lipid rafts in spinal microglia, suggesting alterations in membrane cholesterol dynamics, and increased dimerization of TLR4 (FIGS. 1B and 1C). Intrathecal AIBP (compound 7) reversed CIPN-associated allodynia and normalized lipid raft and TLR4 dimer levels in spinal microglia (FIG. 1A-C). These data suggest that TLR4 receptor dimerization, which is the first step in the activation of a TLR4 inflammatory cascade, occurs in microglial lipid rafts, as is demonstrated in other cell types (Cheng et al., 2012; Zhu et al., 2010). This notion was supported in in vitro experiments in which localization of TLR4 in lipid rafts was significantly increased in BV-2 microglia cells treated with LPS, and AIBP (compound 7) prevented LPS-induced TLR4-CTxB colocalization (FIG. 1D). The specificity of the TLR4 antibodies used in flow cytometry and microscopy experiments was verified with cells from Tlr4^{-/-} mice (FIGS. S1A and B). Since macrophages in the dorsal root ganglia (DRG) are also involved in the nociceptive response and express TLR4, we evaluated their TLR4 dimerization and lipid raft content. However, no significant changes were observed at this time point in the DRG CD11b⁺ myeloid cells (FIGS. S1C and D).

### Short Exposure of AIBP (Compound 7) in CSF and Spinal Cord

A single intrathecal dose of AIBP (compound 7) had a long-lasting therapeutic effect of reversing allodynia in CIPN mice sustained for at least 2 months (Woller et al., 2018). This can be explained either by AIBP (compound 7) long exposure in the spinal cord upon i.t. delivery or by a disease-modifying effect reflected in changes in gene expression profile. To test the former, we measured the pharmacokinetics of AIBP in the CSF and lumbar spinal cord homogenates following i.t. delivery of recombinant AIBP. We used Apoa1bp^{-/-} mice in these experiments to avoid cross-reactivity of the antibodies we use with endogenous mouse AIBP in the spinal cord tissue. This study demonstrated a short AIBP (compound 7) exposure in CSF and spinal cord tissue, with peak levels reached by 30 min and already undetectable after 4 hours (FIGS. 1E and 1F). These results agree with recent reports of rapid clearance of macromolecules from the CSF (Ahn et al., 2019) and suggest that mitigation of TLR4 dynamics in the membrane and possibly other effects of AIBP result in reprogramming of spinal cord microglia and/or other cell types.

### Chemotherapy-Induced Peripheral Neuropathy Alters Gene Expression Profile in Spinal Microglia

To characterize spinal microglia in CIPN, we performed RNA-seq and differential gene expression analysis of the spinal microglia isolated from naïve, cisplatin/saline and cisplatin/AIBP (compound 7) treated wild type (WT) mice (refer to Methods and FIG. S2 for quality control and data set characterization). We used a likelihood ratio test (LRT) to identify genes that were regulated by any condition across all samples and identified 3254 differentially expressed genes (DEGs) that represent the main effect of CIPN and AIBP (compound 7) in spinal microglia transcriptome (FIG. 2A). The majority of these changes were driven by the CIPN condition, with little effect of AIBP (compound 7) (FIGS. 2A and 2B, group 1 and group 2). However, there was a smaller group of CIPN-regulated genes, which changes were completely reversed by AIBP (compound 7) treatment (FIGS. 2A and B, group 3 and group 4). Among the pathways and Gene Ontology (GO) biological processes upregulated in CIPN, were replication, translation and mitochondrial function. Several enriched pathways were related to microglial phenotype changes associated with CNS diseases like Parkinson's and Alzheimer's (FIG. 2C). Cholesterol transporters Abca1 and Abcg1 were downregulated in microglia from cisplatin-treated mice indicating membrane cholesterol trafficking impairment (FIGS. 3A and 3C). Among other down regulated genes were lysosomal genes important in autophagy and lipophagy, which suggest lipid storage dysregulation. The arachidonic acid metabolism genes were also upregulated (FIG. 3C), suggesting the release of bioactive lipid mediators and inflammation.

Using pairwise comparison of CIPN and naïve groups after LRT analysis, we also observed down regulation of Cx3cr1, P2ry12 and Tmem119 homeostatic markers (FIGS. 3A and B), a phenotype related to a transition to neurodegenerative disease-associated microglia (DAM) (Masuda et al., 2019; Nugent et al., 2020; Prinz et al., 2019). A subset of the microglia DAM signature genes reveled a DAM signature with reduction of homeostatic genes and increase of inflammatory and cholesterol metabolism genes. Regulation of the phagocytic TAM receptors Tyrobp, Axl and Mertk partially mimicked DAM characteristic of neurodegenerative disease, except in CIPN microglia we observed downregulation on Trem2 and not a significant effect on its partner receptor gene Tyrobp (FIG. 3B), which suggest a less phagocytic phenotype associated with altered lipid homeostasis and increased lipid accumulation in macroglia and macrophages (Jaitin et al., 2019; Marschallinger et al., 2020).

Interestingly, the part of the DAM signature associated with lipid storage genes that were enriched in CIPN, were downregulated by AIBP (compound 7) (FIGS. 3B and 3C), including the gene encoding the lipid droplet protein PLIN2. The PLIN2 immunohistochemistry validated the RNA-seq results, showing increased number and size of lipid droplets in spinal microglia of cisplatin-treated mice and the reversal of this effect by AIBP (compound 7) (FIG. 3D-H).

### Selective Reversal by AIBP (Compound 7) of CIPN-Induced Changes in Inflammatory Gene Expression

Analyzing the group of genes that were upregulated by CIPN and reversed by AIBP (group 3 in FIG. 2B and FIG. S2F), we found enriched pathways of inflammatory response, leukocyte chemotaxis and neutrophil degranulation pathways (FIG. 4A). Some of the genes in these enriched pathways include Il1b, Ccl2, Glipr1 and Glipr2, Gpnmb, Cxcl2, Cxcl3, S100a8, Il22ra2, Il1r2, Fpr1, Apoe, Ccl9 and the TLR4 interactor gene Tril (FIGS. 4B and C). Examining cytokine protein expression in spinal cord tissue, we confirmed regulation of CCL2 (MCP-1) and CXCL2 (MIP2) expression by AIBP (compound 7) (FIG. 4D). AIBP (compound 7) also downregulated inflammatory and non-inflammatory genes that were not induced by cisplatin. These include Ccl24, Il3ra, Xcr1 and the TLR4 pathway related gene Ptpn22 (FIG. 4E). The pathway and GO analysis of all genes downregulated by AIBP (compound 7) show enrichment in the TLR4 signaling pathway, together with cytokine-cytokine receptor interaction, protein kinase A and C and MAPK regulation pathways, receptor mediated endocytosis and other membrane signaling pathways (FIG. 4F). Regulation of calcium and membrane potential were also downregulated, and enrichment of the peptidase inhibitor pathway shows AIBP (compound 7) effect in recently reported pain associated peptidase inhibition related genes, such as Pi16 and alpha-synuclein gene Snca, which interacts with lipid membranes and regulates vesicle trafficking and neurotransmitter release (FIG. 4G).

Differential expression analysis of microglia from AIBP (compound 7)-treated mice in the CIPN model also revealed 40 genes that were downregulated in CIPN and reversed by AIBP (compound 7) (FIG. S2E). Pathways enriched included regulation of kinase and phosphatase activity (FIG. 4A), and actin cytoskeleton, membrane reorganization and nuclear signaling genes Bin1, Pak1, Vav2 and Ccdc88a and the membrane lipid signaling cascade associated protein Dgka (FIG. 4H). Overall, these results suggest that the AIBP (compound 7) reversal of microglia reprogramming induced in CIPN mice involves regulation of lipid metabolism and trafficking from the membrane to lipid droplets and/or extracellular acceptors.

AIBP (Compound 7) Cannot Reverse Allodynia in Mice with ABCA1/ABCG1 Deficient Microglia

To evaluate the role of microglial cholesterol dynamics in nociception, we first measured colocalization of the ABCA1 cholesterol transporter with the membrane cholesterol accessible for efflux or transport to the endoplasmic reticulum as detected by binding of ALOD4 (He et al., 2017). Treatment of BV-2 cells with LPS decreased colocalization of ALOD4 with ABCA1 and APOA1 in lipid raft domains, and this effect was reversed by AIBP (compound 7) (FIGS. 5A and B).

Next, we generated tamoxifen-inducible, microglia-specific ABCA1 and ABCG1 double knockout mice (ABC-imKO, FIG. 5C) and validated that the ABCA1 and ABCG1 knockdown was observed in spinal cord IBA1⁺ microglia but not in GFAP⁺ astrocytes or NeuN⁺ neurons (FIG. S3). Knocking down ABCA1 and ABCG1 cholesterol transporters in microglia resulted in the development of basal allodynia (day 0), without any stimulatory challenge (FIG. 5D). These results add to the evidence that the impairment of microglial cholesterol trafficking leads to a facilitated state. Indeed, we observed that spinal microglia from naïve ABC-imKO mice had an increased surface expression of TLR4, increased TLR4 dimerization and higher lipid raft content compared to that in WT mice (FIG. 5E).

Remarkably, unlike in WT mice, i.t. AIBP (compound 7) was unable to prevent mechanical allodynia induced by i.t. LPS in ABC-imKO mice (FIGS. 5F and S5A). We then induced CIPN in ABC-imKO mice with cisplatin and observed further rapid onset of allodynia. Furthermore, i.t. delivery of AIBP in ABC-imKO mice at day 7 of the CIPN model did not reverse mechanical allodynia (FIG. 5G), whereas i.t. AIBP was effective in reversing CIPN allodynia in transgenic (littermates) mice treated with vehicle instead of tamoxifen (FIG. 5H) and in wild type mice treated with tamoxifen (FIG. S5B). An i.t. injection of 2-hydroxypropyl-β-cyclodextrin (hp-β-CD), which depletes cholesterol from the plasma membrane but does not require ABCA1 or ABCG1 expression, did alleviate allodynia in ABC-imKO mice (FIG. S4C). In naïve ABC-imKO mice, TLR4 dimerization and lipid raft abundance were significantly higher than in naïve WT mice, and they were not significantly altered by cisplatin nor by AIBP in ABC-imKO microglia (FIGS. 5I and J). These results support the notion that AIBP requires cholesterol transporters to modify inflammarafts and TLR4 dimerization dynamics in microglia and to reverse allodynia.

### ABCA1/ABCG1 Deficiency Reprograms Microglia to a CIPN-Like Phenotype

To understand the effect of cholesterol transport in transcriptional changes induced by CIPN and AIBP, we analyzed differential gene expression in ABC-imKO microglia. We identified 121 genes that changed significantly across two genotypes and three experimental conditions (FIG. S4D). In spinal microglia from naïve, not challenged with cisplatin ABC-imKO mice, most of the upregulated genes and enriched pathways overlapped with the upregulated genes induced by cisplatin in WT mice (FIGS. 6A and B). Among enriched pathways in naïve ABC-imKO mice, we identified response to interferon, inflammatory response, complement activation, and arachidonic acid metabolism pathways (FIG. 6B). Upregulated interferon genes included Ifi207 and Ifi27l2a, and inflammatory genes Xcr1, Cb4, C3, and Klrb1b. Lipid metabolism related genes Apoe and Ch25 h were significantly upregulated in naive ABC-imKO, similar to the changes induced by cisplatin in WT mice (FIGS. 6C and F). This microglia reprogramming might explain, at least in part, the pain behavior observed in naïve ABC-imKO mice

Induction of CIPN in ABC-imKO mice also upregulated several sets of genes and pathways common for both ABC-imKO and WT microglia. However, unlike in WT, such pathways as phagosome, actin dynamics for phagocytic cup formation and cell cycle pathways were not enriched in ABC-imKO microglia of cisplatin-treated mice (FIGS. 6D and S5E). Cisplatin in ABC-imKO microglia failed to induce expression of several inflammatory genes and downregulated expression of Cxcl3, Xrip1, and phagosome related Ferls and Cybb (NOX2) (FIGS. 6F and G). In the absence of cholesterol transporters, cisplatin did not induce cholesterol synthesis pathway genes and downregulated Ch25 h and Dhcr24, suggesting that excess of free cholesterol was present to favor accumulation of desmosterol (FIGS. 6E and G), which is an LXR agonist and a key regulator of macrophage foam cell transcriptome in atherosclerosis (Span et all, 2012). Impaired phagocytosis and upregulation of Tnfrsf26, Trpv4, Il3ra, Il15a, and Shtn1 (FIG. 6E-G) may indicate a differential role of membrane dynamics for nociceptive processes in ABC-imKO mice.

### Microglial Reprogramming by AIBP (Compound 7) is Dependent on ABCA1 and ABCG1 Expression

To understand the differential effect of AIBP (compound 7) in WT and ABC-imKO mice, we compared up and down regulated genes induced by AIBP treatment in both genotypes (FIGS. 7A and B). The effect of AIBP (compound 7) on gene regulation was remarkably different, with only a few common genes downregulated in both genotypes (FIG. 7A). In the absence of cholesterol trafficking machinery, AIBP failed to regulate inflammatory genes and instead induced their expression (FIGS. 7B and C). Induction of inflammatory genes correlates with increased Dhcr24 and other cholesterol biosynthetic genes, including Srebf2, which were downregulated in WT microglia (FIG. 7D). This suggests that reduced desmosterol and increased cholesterol content regulate inflammatory gene expression in microglia. In agreement, induction of genes controlled by LXR, such as Apoe, Apoc1 and Pparg, was observed in microglia of ABC-imKO but not WT mice (FIG. 7D). Other non-inflammatory genes regulated in opposite direction in ABC-imKO by AIBP (compound 7) compared to WT include endopeptidase activity genes Pi16 and Capn11 and the AMPA receptor and synaptic regulator Arc (FIG. 7E). In ABC-imKO microglia, AIBP (compound 7) upregulated cholesterol metabolism pathways, cytokine release and chemokine signaling regulation, kinase and endopeptidase activity and lamellipodia and fiber organization pathways (FIG. 7F). Most of these pathways were downregulated by AIBP in WT microglia (FIG. 4F). Altogether, these data indicate that reprogramming of microglial gene expression induced by AIBP (compound 7) is dependent on cholesterol homeostasis regulated by the cholesterol transporters ABCA1 and ABCG1.

### Microglial AIBP and TLR4 Regulate Nociception

Because the above experiments implicated AIBP (compound 7)-regulated cholesterol homeostasis and activation of microglial TLR4 in nociception, we asked whether microglia-specific knockout of AIBP or TLR4 will affect CIPN allodynia. We generated tamoxifen-inducible, microglia-specific Apoa1bp and Tlr4 knockout mice (AIBP-imKO and TLR4-imKO, FIG. 8A). Knocking down endogenous AIBP in microglia induced mechanical allodynia even before the mice were challenged with cisplatin (day 0, FIG. 8B). This suggests that AIBP plays a role in the maintenance of microglia homeostatic function in mechanical nociception. In control Cx3cr1-Cre^{ERT2} mice with no floxed genes, injections of tamoxifen induced no mechanical allodynia (FIG. 8C). After cisplatin challenge, microglia AIBP knockdown resulted in a faster reduction in mechanical thresholds than in control mice (compare day 2 in FIG. 8D with day 6 in FIG. 8E), indicating higher sensitization in microglia AIBP knockdown mice. Intrathecal injection of recombinant AIBP on day 7 equally reversed CIPN-associated allodynia in both vehicle and tamoxifen-induced AIBP-imKO mice (FIGS. 8C and D). In the whole-body Apoa1bp knockout mice, we did not observe basal allodynia compared to WT mice, and i.t. AIBP rescued CIPN-induced allodynia (FIG. 8F). In contrast to AIBP-imKO or ABC-imKO, the TLR4-imKO mice were protected from the rapid onset of cisplatin-induced allodynia and showed delayed and less severe allodynia (FIG. 8G), suggesting the role of TLR4 expression in microglia in mediating pain sensitization.

### Identification of an AIBP Domain Responsible for TLR4 Binding

Because TLR4-imKO mice were protected from early/acute CIPN (FIG. 8G), we were unable to use this model to evaluate the in vivo significance of AIBP-TLR4 binding we reported previously (Woller et al., 2018). Here we took a different approach and made an AIBP mutant that did not bind TLR4. To elucidate which domain in AIBP is responsible for binding to TLR4, we started from mutating amino acids predicted from the crystal structure (Jha et al., 2008) of the YjeF_N domain of AIBP (FIG. 9A) to participate in protein-protein interaction, but these mutants retained TLR4 binding properties (not shown). Next, we developed a series of deletion mutants of AIBP scanning the full length of the protein and tested them in a pull-down assay with the TLR4 ectodomain (eTLR4) (FIG. 9B). These experiments suggested that a 25-51 amino acid N-terminal domain, located behind the aa 1-24 signal peptide, is involved in eTLR4 binding (FIGS. 9A and B). The aa 25-51 N-terminal domain was unstructured in the published crystal structure of mouse AIBP (Jha et al., 2008). Human and mouse AIBP both contain the homologous aa 25-51 N-terminal domain but zebrafish AIBP does not. Indeed, unlike human and mouse, zebrafish AIBP did not bind human eTLR4 (FIG. 9C). For further experiments, we expressed and purified from a baculovirus/insect cell system wtAIBP (aa 25-288) lacking the signal peptide and mutAIBP (aa 52-288) lacking both the signal peptide and the N-terminal domain.

Unlike wtAIBP, mutAIBP did not bind eTLR4 in a pull-down assay (FIG. 9D) nor in ELISA with eTLR4-coated plates and detection of bound AIBP with the BE-1 anti-AIBP monoclonal antibody (mAb) developed in our lab (Choi et al., 2020) (FIG. 9E). The BE-1 mAb had equal affinity to wtAIBP and mutAIBP (FIG. S5B). Binding of mutAIBP to APOA1 remained unchanged when compared to wtAIBP (FIG. 9F). In cell culture experiments, wtAIBP but not mutAIBP bound to BV-2 microglia stimulated with LPS (FIGS. 9G and H). The increase in wtAIBP binding in response to LPS can be explained by the recruitment of TLR4 to the cell surface and its localization to inflammarafts (Yvan-Charvet et al., 2008; Zhang et al., 2018; Zhu et al., 2010). In aggregate, these results suggest the role of the aa 25-51 N-terminal domain of AIBP in TLR4 binding.

### AIBP Lacking its TLR4 Binding Domain Cannot Alleviate CIPN Allodynia

Unlike wtAIBP, mutAIBP lacking the TLR4 binding site was unable to inhibit LPS-induced TLR4 dimerization in BV-2 microglia (FIG. 10A) but retained the overall ability to reduce lipid rafts (FIG. 10B). Next, we tested the hypothesis that this TLR4 targeting mediates the therapeutic effect of AIBP. Mice that received i.t. saline or mutAIBP prior to i.t. LPS developed allodynia rapidly and to the same extent, whereas i.t. wtAIBP prevented mechanical allodynia induced by LPS (FIG. 10C). In a CIPN mouse model, i.t. wtAIBP reversed established allodynia, with the sustained therapeutic effect lasting for at least 14 days (FIG. 10D). However, i.t. mutAIBP induced only a modest and transient reversal in mechanical thresholds that did not reach naïve or baseline levels and lasted only for 2-3 days (FIG. 10D). At day 21, the mice were terminated, and lumbar spinal cords analyzed. Remarkably, at this late time point, cisplatin-induced polyneuropathy continued to be associated with increased TLR4 dimerization and lipid rafts in spinal microglia, which were significantly reduced by i.t. wtAIBP but not mutAIBP (FIGS. 10E and F), similar to the effect observed on day 8 (FIGS. 1B and C). These results support the hypothesis that AIBP targeting of TLR4 inflammarafts mediates, in large part, the therapeutic effect of AIBP in a mouse model of CIPN.

### Discussion

In this study, we report a new mechanism of selective cholesterol depletion from TLR4-hosting inflammarafts in spinal microglia as a new level of regulation of neuropathic pain in chemotherapy-induced peripheral neuropathy (FIG. 10G) and possibly in other neuropathies. Conditional deletion of cholesterol transporters ABCA1 and ABCG1 in microglia induced spontaneous allodynia in naïve mice showing similarities to the cisplatin effect, and, importantly, the lack of ABCA1 and ABCG1 expression in microglia abolished the AIBP ability to reverse LPS- or cisplatin-induced allodynia or to reduce inflammarafts and TLR4 dimerization in spinal microglia. This differential effect in behavior and TLR4 dynamics was accompanied by the differential gene expression and inability of AIBP to repress inflammatory genes in ABC-imKO microglia.

AIBP has a singular ability to disrupt inflammarafts in activated cells but has little effect on physiological lipid rafts in quiescent cells. We proposed that this is due to AIBP binding to TLR4, which is highly expressed on the surface of inflammatory cells, directing cholesterol depletion to these cells (Miller et al., 2020; Woller et al., 2018). In this work, we identified the N-terminal domain of AIBP as the binding site for TLR4 and demonstrated the critical role of this domain in enabling AIBP binding to activated microglia and its therapeutic effect in CIPN. We propose that this makes AIBP a selective therapy directed to inflammarafts as opposed to non-selective cholesterol removal effected by cyclodextrins, APOA1 and APOA1 mimetic peptides, or LXR agonists. The mutated human AIBP lacking the N-terminal domain still binds to APOA1, and the wild type zebrafish Aibp in which this N-terminal domain is naturally absent, still augments cholesterol efflux from endothelial cells and regulates angiogenesis and orchestrates emergence of hematopoietic stem and progenitor cells from hemogenic endothelium (Fang et al., 2013; Gu et al., 2019), suggesting a different, TLR4-independent mechanism of AIBP interaction with endothelial cells.

Intrathecal delivery of AIBP has a lasting therapeutic effect in a mouse model of CIPN, observed for as long as 10 weeks in our earlier work (Woller et al., 2018) and for 2 weeks in this study. This is in contrast to a short exposure of i.t. AIBP, peaking at 30 min and largely gone by 4 hours from both CSF and lumbar spinal cord tissue. The dissociation between exposure and therapeutic effect suggests a disease-modifying action of AIBP. The reduced CTxB binding and reduced percentage of TLR4 dimers in spinal microglia were observed for as long as 24 hours and even 2 weeks after a single i.t. AIBP injection, indicating sustained disruption of inflammarafts by AIBP, in contrast to their persistent presence in microglia of i.t. saline injected CIPN mice. In addition to the targeted effect on TLR4 inflammarafts, the AIBP disease-modifying effect likely involves reprogramming of gene expression profile in spinal microglia. Although AIBP reversed only 3% of all genes whose expression in spinal microglia was affected by CIPN, AIBP significantly reduced the inflammatory gene expression and the levels of inflammatory cytokines in spinal tissue induced by the cisplatin regimen. These include genes encoding cytokines and chemokines that have been described to have a role in CIPN, such as Il1b, Cxcl2 and Ccl2 (Brandolini et al., 2019; Oliveira et al., 2014; Pevida et al., 2013; Yan et al., 2019).

In addition to inflammatory genes, the cisplatin regimen induced transcriptional changes that resemble the gene signature of diseases associated and neurodegenerative microglia (DAM). CIPN was associated with altered expression of lipid metabolism genes in microglia and the accumulation of lipid droplets, which was reduced by AIBP (compound 7) treatment. A similar microglia lipid droplets phenotype and the transcriptome was recently described as associated with aging and neurodegeneration (Marschallinger et al., 2020; Nugent et al., 2020). Homeostatic genes downregulated during the microglial transition to these pathological phenotypes (Masuda et al., 2019; Nugent et al., 2020; Prinz et al., 2019) were also downregulated in microglia of CIPN mice. Downregulation of microglial Abca1 and Abeg1 expression induced by CIPN is a key factor to understand the AIBP effect. Even though AIBP (compound 7) did not reverse CIPN-associated reduction in Abca1 or Abcg1 mRNA, its ability to stabilize the ABCA1 protein and promote cholesterol efflux (Zhang et al., 2016) might suffice to normalize microglia cholesterol metabolism. The AIBP (compound 7) effect on allodynia was replicated, albeit transiently, by i.t. APOA1 or an LXR agonist (Woller et al., 2018). Furthermore, a negative association of an ABCA1 single nucleotide variant has been found with quality of life scores in painful bone metastasis patients (Furfari et al., 2017). However, we cannot exclude other mechanisms, unrelated to the reversal of a subset of CIPN-affected genes, by which AIBP (compound 7) reprograms microglia to confer a protective phenotype in facilitated pain states.

One of the key findings of this work was that in the absence of ABCA1 and ABCG1 in microglia, AIBP failed to downregulate inflammatory genes and even upregulated some of them and upregulated non-inflammatory, pain-related Arc, and Pi16 genes that regulate synaptic plasticity (Hossaini et al., 2010; Singhmar et al., 2020). Differential reprograming by AIBP of WT and ABCA1/ABCG1-deficient microglia could be dependent on the desmosterol converting enzyme Dher24, which regulates desmosterol and cholesterol content and when decreased is associated with foam cell formation and homeostatic anti-inflammatory response (Spann et al., 2012). Importantly, AIBP (compound 7) was also unable to reverse CIPN or LPS induced allodynia in ABC-imKO mice. These results indicate that the AIBP anti-inflammatory and anti-nociceptive effects depend on cholesterol depletion from the plasma membrane and that in the absence of efflux machinery, AIBP may in fact promote inflammatory and cytotoxic effects.

Overall, the results of this study suggest that regulation of cholesterol content in the plasma membrane of spinal microglia has profound effects on the cell signaling originating from inflammarafts and the ensuing gene expression of inflammatory and lipid metabolism genes, culminating in control of nociception under polyneuropathy conditions.

### Materials and Methods

Animals. Wild type, Abca1^{fl/fl} Abcg1^{fl/fl}, Tlr4^{fl/fl}, Slc1a3-Cre^{ERT} and Cx3cr1-Cre^{ERT2} mice, all on the C57BL/6 background, were purchased from the Jackson Lab (Bar Harbor, ME) or bred and weaned in-house. Tlr4^{-/-} mice were a gift from Dr. Akira (Osaka University). The Apoa1bp^{fl/fl} mouse was previously generated in our laboratory using ES cells derived from C57BL/6 mice. The following mouse lines were cross-bred in our laboratories: Apoa1bp^{fl/fl} Cx3cr1-Cre^{ERT2} (AIBP-imKO), Tlr4^{fl/fl} Cx3cr1-Cre^{ERT2} (TLR4-imKO), Abca1^{fl/fl} Abcg1^{fl/fl} Cx3cr1-Cre^{ERT2} (ABC-imKO), and Abca1^{fl/fl} Abcg1^{fl/fl} Slc1a3-Cre_{ERT} (ABC-iaKO). All microglia conditional knockout mice used in experiments had only one allele of Cx3cr1-Cre^{ERT2} to avoid generating a Cx3cr1 knockout. Mice were housed up to 4 per standard cage at room temperature and maintained on a 12:12 hour light:dark cycle. All behavioral testing was performed during the light cycle. Both food and water were available ad libitum. All experiments were conducted with male mice and according to protocols approved by the Institutional Animal Care and Use Committee (IACUC) of the University of California

Cells. BV-2 immortalized microglia cell line (Blasi et al., 1990) was cultured in Dulbecco's MEM with 5% fetal bovine serum (FBS). Thioglycollate-elicited peritoneal macrophages were harvested from C57BL/6 or Tlr4^{-/-} mice and maintained in DMEM (Cellgro) supplemented with 10% heat-inactivated FBS (Cellgro) and 50 μg/mL gentamicin (Omega Scientific). HEK293 cells (RRID:CVCL_0045) were cultured in DMEM supplemented with 10% FBS and 50 μg/mL gentamicin. All cells were cultured in 5% CO₂ atmosphere at 37° C. Cell lines were used between passages 1-3.

Chemotherapy-induced peripheral neuropathy model. To develop chemotherapy-induced peripheral neuropathy (CIPN), intraperitoneal (i.p.) injections of cisplatin (2.3 mg/kg/injection; Spectrum Chemical MFG) were performed on day 1 and day 3. During the period of cisplatin administration, weight loss, behavioral changes and mechanical allodynia were monitored and measured. The criteria for euthanasia were the weight loss in excess of 20% body weight and erratic behavior; however, no animals required euthanasia.

Mechanical allodynia measurements. Animals were placed in clear, plastic, bottomless cages over a wire mesh surface and acclimated for at least 30 min prior to the initiation of testing. Tactile thresholds were measured with a series of von Frey filaments (Bioseb) ranging from 2.44-4.31 (0.02-2.00 g). The 50% probability of withdrawal threshold was recorded. Mechanical withdrawal thresholds were assessed prior to treatment (baseline or day 0) and at enter time points post-treatment using the up-down method (Chaplan et al., 1994).

Intrathecal delivery of AIBP (compound 7) or saline. Mice were anesthetized using 5% isoflurane in oxygen for induction and 2% isoflurane in oxygen for maintenance of anesthesia. Intrathecal injections were performed according to (Hylden and Wilcox, 1980). Briefly, the lower back was shaven and disinfected, and the animals were placed in a prone posture holding the pelvis between the thumb and forefinger. The L5 and L6 vertebrae were identified by palpation and a 30G needle was inserted percutaneously on the midline between the L5 and L6 vertebrae. Successful entry was assessed by the observation of a tail flick. Injections of 5 μL were administered over an interval of ˜30 seconds. Drugs for intrathecal delivery were formulated in physiological sterile 0.9% NaCl. Based on previous study (Woller et al., 2018), AIBP (compound 7) dosing for spinal delivery in these studies was 0.5 μg/5 μL. Following recovery from anesthesia, mice were evaluated for normal motor coordination and muscle tone.

Intraperitoneal injection of tamoxifen for inducible Cre-driver lines. In this study we followed Jackson Lab tamoxifen induction protocol. Tamoxifen (Sigma-Aldrich) was dissolved in corn oil at a concentration of 10 mg/mL by shaking overnight at 37° C. and wrapped in aluminum foil and stored at 4° C. 200 μL tamoxifen or vehicle (corn oil) were injected intraperitoneally every 24 hours for 5 consecutive days.

Ex-vivo and in vitro TLR4 dimerization and lipid rafts assays. The TLR4 dimerization assay uses two TLR4 antibodies for flow cytometry: MTS510 recognizes TLR4/MD2 as a monomer (in TLR4 units) but not a dimer; SA15-21 binds to any cell surface TLR4 irrespective of its dimerization status (Akashi et al., 2003; Zanoni et al., 2016). The percentage of TLR4 dimers was then calculated from MTS510 and SA15-21 measured in the same cell suspension. Lipid raft content was measured using CTxB, which binds to ganglioside GM1. To assess TLR4 dimerization in vitro, BV-2 cells were preincubated with 0.2 g/ml AIBP (compound 7) in serum-containing medium for 30 min, followed by a 15 min incubation with LPS 100 ng/mL. At the end of incubation, cells were immediately put on ice, washed once with PBS and fixed for 10 min with 4% formaldehyde. Then cells were washed two times with ice cold FACS buffer, incubated with 2% normal mouse serum containing an anti-CD16/CD32 antibody (FcγR blocker, BD Bioscience) for 30 min on ice, followed by staining with a 1:100 dilution of PE-conjugated MTS510 antibody and an APC-conjugated SA15-21 antibody (ThermoFisher and Biolegend respectively, RRID: AB_2562503 and RRID: AB_466263) together with 1:200 dilution of CTxB-FITC (ThermoFisher) for 30 min on ice. Cells were washed and analyzed using a FACSCanto II (BD Biosciences) flow cytometer.

For ex vivo assays, spinal cords were harvested by hydro extrusion (Kennedy et al., 2013), fixed with 4% formaldehyde and put on ice while processing. Single-cell suspensions from lumbar tissue were obtained using a Neural Tissue Dissociation kit (Miltenyi Biotec) according to the manufacturer's protocol. To remove myelin, Myelin Removal Beads II (Miltenyi Biotec) were added to samples and incubated for 15 min at 4° C., followed by separation with LS column and a MACS Separator (Miltenyi Biotec). Following isolation, cells were incubated with 2% normal mouse serum containing an anti-CD16/CD32 antibody (FcγR blocker, BD Bioscience) for 30 min on ice, followed by staining with an antibody mix of 1:100 PerCP-Cy5.5-conjugated CD11b antibody (Biolegend, RRID:AB_893232), 1:100 rabbit anti-mouse TMEM119 antibody (Abcam, RRID:AB_2744673), PE-conjugated MTS510, APC-conjugated SA15-21 antibodies (ThermoFisher, RRID:AB_2562503 and Biolegend, RRID:AB_466263 respectively) and 1:200 dilution of CTxB-FITC (ThermoFisher) for 45 min on ice, cells were then washed and incubated with (1:250) secondary Alexa PECy7 conjugated anti-rabbit antibody for 30 min on ice. Cells were washed and analyzed using a FACSCanto II (BD Biosciences) flow cytometer.

For in vitro and ex-vivo staining compensations beads and/or single stained cells we use to compensate the signal overlap between channels and isotype controls for CD11b, MTS510 and SA15-21 antibodies together with FMO were used to delineate gates. Data was analyzed by FlowJo (BD Bioscience, RRID: SCR_008520). From these data, we calculated the abundance of lipid rafts and a relative change in the number of TLR4 dimers in spinal microglia (zero dimers were arbitrarily assigned to unstimulated or naïve cells).

Immunofluorescence, confocal imaging and colocalization analysis. BV-2 cells were plated on coverslips in 12-well plates and preincubated with 0.2 μg/ml AIBP in 5% serum-containing medium for 30 min, followed by a 5- or 15-min incubation with 100 ng/mL LPS. At the end of incubation, cells were immediately put on ice, washed once with PBS and fixed for 10 min with 4% formaldehyde. Cells were washed two times with ice-cold PBS, incubated with blocking buffer containing 5% FBS for 30 min, followed by staining with a 1:200 dilution of CTxB-Alexa555 and 1:100 dilution of mouse anti-TLR4 antibody (Abcam, RRID:AB_446735), or with 1:100 rabbit anti-APOA1 antibody (Abcam) or 1:100 rabbit anti-ABCA1 (Novus Biological RRID:AB_10000630), washed and incubated with anti-rabbit Alexa 647 conjugated secondary antibody and incubated with recombinant, His-tagged ALOD4 and a 1:100 FITC-conjugated anti-His secondary antibody (LSbio) for staining of accessible cholesterol in the membrane. Cells were washed and coverslips were mounted with Prolong Gold into slides and sealed. Slides were analyzed using a Leica SP8 super resolution confocal microscope with Lightening deconvolution or STED.

For validating microglia-specific AIBP or ABCA1/ABCG1 knockout, spinal cord tissue was collected and post fixed in 4% formaldehyde at 4° C. Then the tissue was dehydrated in 30% sucrose and frozen in OCT until sectioning. Spinal cords were sliced into 10 μm sections using a cryostat, and slides were store at −20° C. Frozen sections were blocked with a 2% FBS and 0.3% Triton X100 solution, followed by incubations with 1:100 rabbit anti-AIBP antibody (a kind gift from Dr. Longhou Fang). Separate sections were stained with 1:100 rabbit anti-ABCA1 or 1:100 rabbit anti-ABCG1 antibodies (Novus Biological, RRID:AB_10000630 and RRID:AB_10125717) overnight at 4° C. Slides were washed and incubated with a 1:200 dilution of anti-rabbit Alexa488 (Abcam, RRID:AB_2630356) or Alexa647 conjugated secondary antibody for 2 hours, followed by 3 washes and all sections were incubated with either Alexa488 conjugated IBA-1 antibody (Milipore-Sigma) or Alexa633 conjugated IBA1 antibody (Wako Chemicals, RRID: AB_2687911). Alternatively, slides were incubated with either 1:100 Alexa488 conjugated anti-NeuN antibody (Cell Signaling, RRID:AB_2799470) or 1:100 Alexa488 conjugated anti-GFAP antibody (Cell Signaling, RRID:AB_2263284). Slides were washed 3 times with PBS and mounted with Prolonged Gold with DAPI (Cell Signaling). Image acquisitions of at least one slide of each animal were performed using a 63× objective and a Leica SP8 confocal microscope with Lightening deconvolution. Colocalization analyses were performed in ImageJ/FIJI (NIH, RRID:SCR_003070/SCR_002285) using Coloc2 tool. Thresholds, Pearson's R and Manders' coefficients above thresholds, together with masked colocalized mages, Costes P value and pixel scatter plots were generated for each image. tM1 or tM2 were used depending on which channel represented the cell markers.

ALOD4 expression and purification. The pALOD4 plasmid (Gay A., 2015) was obtained from Addgene (Cat no #111026, RRID:Addgene_111026) and used to transform E. coli competent cells BL21(DE3), and positive colonies were selected in Amp⁺ LB plates. After induction of the expression with 1 mM isopropyl β-d-1-thiogalactopyranoside (IPTG) and lysis, His-tagged ALOD4 was purified using an Ni-NTA agarose column with imidazole elution. Protein was dialyzed against PBS and concentration measured. Aliquots were stored at −80° C.

Cloning and expression of wtAIBP and mutAIBP in baculovirus/insect cell system. AIBP (compound 7) was produced in a baculovirus/insect cell system to ensure posttranslational modification and endotoxin-free preparation as described in (Choi et al., 2018; Woller et al., 2018). Human wild type (wt) AIBP and mutant (mut) AIBP, mouse wild type AIBP, and zebrafish wild type AIBP (Fang et al., 2013) were cloned into a pAcHLT-C vector behind the polyhedrin promoter. The vector contains an N-terminal His-tag to enable purification and detection. Insect Sf9 cells were transfected with BestBac baculovirus DNA (Expression Systems) and the AIBP vector. After 4-5 days, the supernatant was collected to afford a baculovirus stock. Fresh Sf9 cells were infected with the AIBP producing baculovirus, cell pellets were collected after 3 days, lysed, sonicated, cleared by centrifugation, and the supernatants loaded onto a Ni-NTA agarose column eluted with imidazole. Protein was dialyzed against saline, and concentration measured. Aliquots were stored at −80° C.

Pharmacokinetics of AIBP (compound 7) in spinal tissue. Knockout AIBP mice were used for the pharmacokinetic study. Intrathecal injections of AIBP (2.5 μg/5 μL) were performed as previously described (Hylden and Wilcox, 1980), and the CSF was collected after 15 min, 30 min, 1 h, 4 h or 8 h, as described (Liu and Duff, 2008). Briefly, capillary tubes (0.8×100 mm) were pulled using a micropipette puller. Mice were anesthetized using 3% isoflurane with mixture of 50% oxygen and 50% room air. The skin of the neck was shaved, and the mouse was placed on the stereotaxic instrument. After swabbing the surgical site, a sagittal incision of the skin inferior to the occiput was made. The subcutaneous tissue and muscles were dissected away exposing the dura mater. The pulled capillary tube was directly punctured into the cisterna magna, and non-contaminated sample was drawn. After CSF collection, the capillary tube was flushed into a PCR tube containing 50 μL of NaCl 0.09% and the mouse was then perfused with 35 mL of 0.9% NaCl. The spinal cord was flushed by hydro-extrusion with 5 mL of 0.9% NaCl. Spinal cord tissue was weighted and extracted with complete N-PER™ Neuronal Protein Extraction Reagent (Thermo Fisher) at 1 g/10 mL on ice. After 10 min incubation on ice, samples were centrifuged (10,000×g for 10 min at 4° C.) to pellet the cell debris, and supernatants were diluted 1:1 with 1% BSA-TBS. Plates were coated with BE-1 anti-AIBP monoclonal antibody (5 μg/mL), incubated for 3 h with spinal cord extracts or CSF samples and detected with a rabbit polyclonal anti-AIBP antibody, followed by a goat-anti-rabbit-ALP antibody (Sigma Aldrich, RRID: AB_258103). Plates were read as above.

FACS sorting of spinal microglia for RNA-seq. Cell suspensions from lumbar spinal cords were prepared as described above, except the fixation step. Fresh tissue was processed and blocked for 30 min with 2% normal mouse serum containing an anti-CD16/CD32 antibody (FcγR blocker, BD Bioscience) and then stained with a mix of 1:50 PE-Cy7-conjugated CD11b antibody (Biolegend, RRID: AB_312799), 1:50 rabbit anti-mouse TMEM119 antibody (Abcam, RRID: AB_2744673), 1:50 PerCP-Cy5.5-conjugated CD24 antibody (Biolegend, RRID:AB_1595491), cells were then washed and incubated with (1:200) secondary Alexa488 conjugated anti-rabbit antibody (Abcam, RRID:AB_2630356) and incubated for 30 min on ice, after that cells were washed and incubated with 1:50 Alexa 647-conjugated Glast1 antibody (Novus Biologicals) and 1:100 dilution of Life/Death Ghost Red 780 dye (Cell Signaling) for 30 min on ice. Cells were washed with sorting buffer and filtered before being sorted into lysis buffer, using a BD FACS-Aria cell sorter (BD Biosciences). Three technical replicates, each with 400 cells, from the same animal were sorted. See FIGS. S2A and B for sorting strategy and analysis of purity of sorted microglia.

RNA-seq library prep, sequencing and quality control. We followed the low input bulk seq SmartSeq2 protocol from (Rosales et al., 2018). Cells sorted into a lysis buffer containing Triton X-100, RNase Inhibitor, and Oligo(dT)30-VN were hybridized with oligo(dT)+ to the poly(A) tails of the mRNA. Reagents for reverse transcription were added to construct cDNA libraries following addition of reagents for PCR amplification (qPCR was not performed at this point). Libraries were quantified and QC was performed using TapeStation high sensitivity D5000 screentape in addition to Qubit double stranded high sensitivity assay. All samples were adjusted to 1ng of cDNA for input into NexteraXT protocol. QC check was performed with TapeStation high sensitivity D1000 screen tape in addition to Qubit double stranded high sensitivity assay. The samples were subjected to qPCR and pooling and loaded onto the NovaSeq for paired-end 50×50 reads using NovaSeq S1 100 cycle kit.

Splice aware alignment of FASTQ data was done using STAR (Dobin et al., 2013), Quality control of sequenced data and alignment was performed by FASTQC (RRID:SCR_014583), QoRTs (RRID:SCR_018665) (Hartley and Mullikin, 2015) and MultiQC tool (RRID:SCR_014982) (Ewels et al., 2016). Counting of genes associated with the reads was performed using STAR (RRID:SCR_015899).

Sequencing Quality controls indicated good data quality (MultiQC reports). Two technical replicates (Y_10 and Y_30) were removed due to suboptimal gene coverage. We used R package, DEseq2 (RRID:SCR_015687) to analyze differential expression (Love et al., 2014). We identified a total of 18,818 genes in lumbar spinal microglia with cut-off set to more than 10 counts per million mapped reads CPM for at least 3 samples. One sample was removed from further analysis because it displayed extreme irregular distribution in PCA and clustering in the top 500 most variable genes in comparison to all other samples. We used a subset of 40 microglia-specific genes reported in (Butovsky et al., 2014) and the genes specific for neurons (Nefl), oligodendrocytes (Omg) and astrocytes (Slc6a1) to confirm the microglia enrichment in our samples and data (FIG. S2D). Determination of DEGs was performed by DEseq2 binomial modeling using a likelihood ratio test (LRT) including all samples across all factors and using a reduced design without condition factor to determine the main effect of cisplatin and AIBP and all significant genes altered by these conditions. We used an LRT model comparing to a reduced design without interaction term of condition and genotype to identify the genes regulated in a genotype (ABC-imKO) dependent manner. Adjusted P<0.05 and an FDR of 5% were used to filter significant genes. Determination of clusters of genes by gene expression pattern of the identified significant genes was performed by DESeq2 function: degpatterns. Pairwise comparisons after LRT of experimental groups were performed with Wald test and accounting for an FDR of 5%. Volcano plots include genes significantly different with an absolute fold change>1.5. Pathway enrichment and GO analysis were performed in metascape.org (RRID:SCR_016620) using a minimum of 3 genes and P<0.05 (Zhou et al., 2019).

Co-immunoprecipitation assays for TLR4 binding. Pull down assay of eTLR4 and wtAIBP or mutAIBP in test tube was performed by mixing 1 μg of eTLR4 (Sino Biological) and AIBP in PBS containing 0.5% Triton X-100 and incubating for 1 hour at room temperature. Samples were precleared by adding Protein A/G Sepharose beads at room temperature for 30 min, followed by addition of 1 μg of BE-1 monoclonal anti-AIBP antibody and incubation for 2 hours. Protein A/G Sepharose beads were added and incubated for an additional one hour, followed by 5 washes with PBS containing 0.5% Triton X-100 and immunoblot of samples.

HEK293 cells (RRID:CVCL_0045) were transfected with Flag-eTLR4 and a Flag-AIBP (wild type or one of the mutants) construct. Thirty-six hours after transfection, cells were harvested and lysed with an ice-cold lysis buffer (50 mM Tris-HCl, pH7.5, 1% NP-40, 150 mM NaCl, 1 mM EDTA, 1 mM EGTA, 5 mM Na3VO4, 1 mM NaF, and a protease inhibitor cocktail from Sigma). Cell lysates were preincubated with protein A/G Sepharose beads for 30 min at 4° C. and immunoprecipitated with a mouse anti-TLR4 antibody (Abcam) overnight at 4° C. Next day, the lysates were incubated with protein A/G beads for 1 hour at 4° C. Unbound proteins were removed by washing with lysis buffer, and the beads were run on a Bolt Bis-Tris gel (Invitrogen); the bound AIBP was detected by immunoblotting with an anti-Flag antibody (Sigma).

ELISA binding assays. To assess AIBP-TLR4 binding, 96-well plates were coated with 5 μg/ml of eTLR4, washed three times with PBS containing 0.05% Tween-20, blocked with PBS containing 1% BSA, and incubated with wtAIBP or mutAIBP, followed by 2 μg/ml of a biotinylated BE-1 anti-AIBP monoclonal antibody. To assess AIBP-APOA1 binding, plates were coated with BSA, wtAIBP or mutAIBP, washed, blocked, and incubated with 5 μg/ml of human APOA1 (a gift from Dmitri Sviridov, Baker Heart and Diabetes Institute, Melbourne, Australia), followed by a biotinylated anti-APOA1 antibody (Academy Bio-Medical Company, RRID:AB_1238781). In both assays, neutravidin-AP was added and incubated for 45 minutes at room temperature, followed by LumiPhos 530 (Lumigen) for 90 min, and luminescence was measured using a luminescence plate reader (BioTek, Winooski, Vermont).

Flow cytometry assay for AIBP cell binding. BV-2 microglia cells stimulated or not with 100 ng/mL LPS for 15 min were blocked with Tris-buffered saline (TBS) containing 1% BSA for 60 min on ice and incubated with either 2 μg/mL BSA or 2 μg/mL AIBP for 2 h on ice. Cells were fixed and incubated with 1 μg/mL FITC-conjugated anti-His antibody (LSBio) for 1 h at 4° C. and analyzed using a FACSCanto II (BD Biosciences) flow cytometer and FlowJo software (RRID: SCR_008520).

Cytokine measurement in spinal tissue by ELISA. Levels of IL-6 (DY406), IL-1B (DY401), MCP-1 (DY479) and MIP2 (DY452) in spinal cord lysates were measured using a mouse DuoSet ELISA (R&D Systems) according to the manufacturer's instructions.

Statistical analyses. For other than RNAseq data sets, results were analyzed using Student's t-test (for differences between 2 groups), one-way ANOVA (for multiple groups), or two-way ANOVA with the Bonferroni post hoc test (for multiple groups time course experiments), using GraphPad Prism (RRID:SCR_002798). Differences between groups with P<0.05 were considered statistically significant.

### Figure Legends

FIG. 1 demonstrates reversal of pain behavior by wild type (wt) AIBP protein in a mouse model of chemotherapy-induced peripheral neuropathy (CIPN) and a reduction of activated TLR4 dimers associated with pro-inflammatory lipid rafts (Inflammarafts):

FIG. 1. Chemotherapy-induced peripheral neuropathy alters TLR4 dimerization and lipid rafts in spinal microglia: reversal by AIBP. A, Withdrawal thresholds in WT mice in response to i.p. cisplatin (2 injections of 2.3 mg/kg/day), followed by a single dose of i.t. saline (5 μl) or AIBP (compound 7) (0.5 μg/5 μl). Naïve mice received no injections. Data from 2 independent experiments (n=6 per group) Data from 2 independent experiments. B-C, Analysis of CD11b⁺/TMEM119⁺ spinal microglia cells showing TLR4 dimerization (B) and lipid raft content measured by CTxB staining (C) 24 hours after i.t. saline or AIBP, i.e. at day 8 of the time course shown in A. Data from 3 independent experiments (n=9 per group for TLR4 dimerization and n=12 for lipid raft staining). D, BV-2 microglia cells were incubated for 30 min with AIBP (compound 7) (0.2 μg/mL) or vehicle in complete media, followed by a 5 min incubation with LPS (100 ng/mL). Scale bar, 5 μm. Bar graph shows Manders' tM1 coefficient. E-F, Pharmacokinetics of i.t. AIBP (2.5 μg/5 μL) in male Apoa1bp^{-/-} mice in CSF (E) and lumbar spinal cord (F) (n=5). *, P<0.05;**, P<0.01;***, P<0.001. Two-way ANOVA with Bonferroni post-hoc test for multiple comparisons in grouped analyses; one-way ANOVA with Tukey post-hoc test for multiple comparisons of 3 groups and imaging quantification.

FIG. 2 compares the change in gene signature of naive mice to those treated with chemotherapy agent cisplatin to mice treated with cisplatin and a wild type (wt) AIBP protein:

FIG. 2. Gene expression in spinal microglia of CIPN mice. A-B, Microglia (CD11b⁺TEMEM119⁺) were FACS-sorted from 3 groups shown in FIG. 1A: WT naïve or injected with cisplatin (days 1 and 3), followed on day 7 by i.t. saline (5 μL) or AIBP (compound 7) (0.5 μg/5 μL), and terminated on day 8, and subjected to RNA-seq; n=3 biological replicates (mice) for naïve and cisplatin/saline and n=2 for cisplatin/AIBP (each biological replicate was collapsed from 3 technical replicates from the same animal). A, Heatmap of DEGs across all samples (all technical replicates are presented in columns). Significant (adjusted P<0.01) up or down regulated genes showing main effect tested by LRT (likelihood ratio test). Log 2 relative expression, B, Groups of significant DEGs clustered based on expression profile patterns in different treatment conditions. C, Pathway and GO enrichment analysis of upregulated (group1 in panel 2B) and downregulated (group2) genes induced by cisplatin treatment, using adjusted P<0.05 and absolute fold change>1.5 and a minimum overlap of 3 genes in the pathway. Upregulated pathways are shown in red and downregulated in blue.

FIG. 3 compares differences in disease associated microglia (DAM) gene expression signature and lipid droplets in mice receiving chemotherapy to naïve mice and CIPN mice treated with a wt AIBP:

FIG. 3. DAM and lipid related gene expression and lipid droplets in spinal microglia of CIPN mice. A-C, Same groups as in FIG. 2. A, Volcano plot of upregulated and downregulated genes in spinal microglia of cisplatin-treated vs. naïve mice. Cutoff of adjusted P<0.05 and absolute fold change>1.5 represented in light green dots. B, Heatmap depicting disease associated microglia (DAM) signature genes. C, Heatmap of log 2 normalized gene counts scaled by row showing lipid related gene sets. D-H, Lipid droplet accumulation in spinal microglia measured by PLIN2 immunostaining in spinal cord sections co-stained with IBA1 and DAPI. Experimental conditions as in FIG. 1A; n=5 fields of view from 5 mice per group from 2 independent experiments. Scale bar, 20 μm. Mean±S.E.M.; *, P<0.05 comparing to naïve group, tested by one-way ANOVA with Tukey's test for multiple comparisons in grouped analyses.

FIG. 4 summarizes changes in gene expression in CIPN mice that have been treated with a wt AIBP protein:

FIG. 4. Gene expression in spinal microglia of CIPN mice: effect of AIBP (compound 7). Experimental conditions and analysis as in FIG. 1; n-2-3 biological replicates per group (each biological replicate collapsed from 3 technical replicates). A, pathway and GO enrichment analysis of CIPN-upregulated genes that were downregulated by AIBP (compound 7) (group 3 in FIG. 2B)) and CIPN-downregulated genes that were upregulated by AIBP (compound 7) (group 4), using adjusted P<0.05 and absolute fold change>1.5 and a minimum overlap of 3 genes in the pathway. Upregulated pathways are shown in red and downregulated in blue. B, DEGs in spinal microglia induced by i.t. AIBP. Adjusted P<0.05 and Benjamini-Hochberg FDR<5% represented in a volcano plot of up and down regulated genes in cisplatin/AIBP vs. cisplatin/saline treated mice. Cutoff adjusted P<0.05 and absolute fold change>1.5 shown in light green dots. C, Heatmap of inflammatory genes in group 3 upregulated in CIPN and downregulated by AIBP. D, Cytokine protein expression in spinal tissue from WT naïve, cisplatin/saline and cisplatin/AIBP groups; n=5 per group. E, Heatmap of inflammatory genes not induced by cisplatin but downregulated by AIBP (compound 7). F, Pathway and GO enrichment analysis of all genes downregulated by AIBP (compound 7) using adjusted P<0.05 and absolute fold change>1.5 and a minimum overlap of 3 genes in a pathway. G, Heatmap of non-inflammatory genes downregulated by AIBP (compound 7) included in the most enriched pathway: peptidase inhibitor activity pathway. H, Heatmap of genes whose downregulation in CIPN was reversed by AIBP (compound 7). Mean±S.E.M.; *P<0.05 comparing to naïve group and cisplatin/i.t. saline group.

FIG. 5 demonstrates that the cholesterol transporters ABCA1 and ABCG1 are necessary for AIBP-mediated reversal of pain in a model of mouse CIPN:

FIG. 5. ABCA1 and ABCG1 expression in microglia controls nociception and is required for AIBP (compound 7)-mediated reversal of allodynia in a mouse model of CIPN. A-B, BV-2 cells were incubated for 30 min with AIBP (compound 7) (0.2 μg/mL) or vehicle in complete media, followed by a 5 min incubation with LPS (100 ng/mL). Colocalization of accessible cholesterol with ABCA1 (A) and APOA1 (B) in lipid rafts. Scale bar, 7 μm. Bar graphs show Manders' tM1 coefficient. C, Experimental design and timeline: Tamoxifen (TAM, 10 mg/mL, 200 μL/day), cisplatin (2.3 mg/Kg), AIBP (compound 7) (0.5 μg/5 μl) or saline (5 μl). D, Baseline (day 0) withdrawal thresholds before the start of cisplatin intervention. Data from 3 independent experiments (n=8 for vehicle treated ABC-imKO mice, n=16 for TAM treated ABC-imKO mice and n=15 for littermates Abca1^{fl/fl} Abcg1^{fl/fl} no-Cre [WT] mice treated with TAM). E, TLR4 surface expression and dimerization and lipid rafts (CTxB) in CD11b⁺TMEM119⁺ spinal microglia of naïve WT and ABC-imKO mice at baseline (day 0) (n=5 for TLR4 surface expression and lipid raft content analysis for both groups, n=8 for WT and n=9 for ABC-imKO for TLR4 dimerization). F, Withdrawal thresholds after i.t. saline or AIBP (compound 7) (0.5 μg/5 μl), followed by i.t. LPS (0.1 μg/5 μl) in TAM-induced ABC-imKO mice (n=4 per group). G-H, Withdrawal thresholds following i.p. cisplatin and i.t. saline or AIBP (0.5 μg/5 μl) injections in TAM-induced ABC-imKO (G) and non-induced (vehicle) ABC-imKO (H) mice (n=6 per group); data from 2 independent experiments I-J, TLR4 dimerization (I) and lipid rafts (J) in CD11b⁺TEMEM119⁺ spinal microglia at day 8 in the groups shown in panels G and H. Mean±S.E.M. (n=7-8) from 2 independent experiments. *, P<0.05;***, P<0.001. Two-way ANOVA with Bonferroni post-hoc test for multiple comparisons in time-course analysis; t-test for 2 groups, and one-way ANOVA with Tukey post-hoc test for multiple comparisons of more than 2 groups.

FIG. 6 characterizes gene expression in ABC gene knockout mice:

FIG. 6. Gene expression in spinal microglia of ABC-imKO mice. Microglia (CD11b⁺TEMEM119⁺) were FACS-sorted from 3 groups of ABC-imKO mice: naïve or injected with cisplatin (days 1 and 3), followed on day 7 by i.t. saline (5 μL) or AIBP (compound 7) (0.5 μg/5 μL), and terminated on day 8; n=3 biological replicates (each biological replicate collapsed from 3 technical replicates). RNA-seq data sets from ABC-imKO and WT (not littermates) mice were acquired in the same experiment. A, Top: Overlapping genes and pathways induced in naïve ABC-imKO microglia and shared with WT microglia in mice treated with cisplatin, showed in purple lines connecting overlapping genes and in blue lines connecting the overlapping enriched pathways. Bottom: Venn diagram of upregulated genes in spinal microglia from WT cisplatin and ABC-imKO naïve mice. B, Enrichment pathway analysis of up and down regulated genes induced by ABCA1 and ABCG1 knockdown in microglia, using cutoff P<0.05, enrichment>1.5 and a minimum overlap of 3 genes in the pathway. C, DEGs in naïve spinal microglia of TAM-induced ABC-imKO mice. Adjusted P<0.05 and Benjamini-Hochberg FDR<5%. D, Overlapping genes and pathways induced by cisplatin treatment in ABC-imKO microglia and shared with WT microglia in mice treated with cisplatin. E, DEGs in spinal microglia of cisplatin-treated, TAM-induced ABC-imKO mice compared to cisplatin-treated WT mice. Adjusted P<0.05 and Benjamini-Hochberg FDR<5%. F-G, Heatmap of DEGs upregulated (F) or downregulated (G) in ABC-imKO microglia either in naïve or cisplatin condition.

FIG. 7: Compares gene expression of wild type and ABC knockout mice treated with an AIBP protein (compound 7) as provided herein:

FIG. 7. Microglial reprogramming by AIBP is dependent on ABCA1/ABCG1 expression. A, Venn diagram comparing the effect of AIBP treatment on gene expression in WT and ABC-imKO mice in which CIPN was induced by cisplatin. B, Volcano plot representation of up and down regulated genes by AIBP treatment in CIPN comparing AIBP effect on ABC-imKO vs. WT mice. Cutoff of adjusted P<0.05 and absolute fold change>1.5 shown in light green dots. C, Heatmap of log 2 normalized gene counts of inflammatory genes altered by AIBP in an ABC-dependent manner (downregulated by AIBP in WT microglia but upregulated by AIBP in ABC-imKO. D, Heatmap of cholesterol synthesis and LXR related genes comparing cisplatin and AIBP effect in wild type and ABC-imKO. E, Heatmap of non-inflammatory genes regulated by AIBP in an ABC-dependent manner. F, Enrichment pathway analysis of upregulated genes by AIBP in ABC-imKO microglia, using cutoff P<0.05, enrichment>1.5 and a minimum overlap of 3 genes in the pathway.

FIG. 8 demonstrates that knockout of either AIBP or TLR4 contributes to pain behavior (nociception):

FIG. 8. Endogenous AIBP and TLR4 in microglia are important in nociception. A, Experimental design and timeline. Tamoxifen (TAM, 10 mg/mL, 200 μL/day); cisplatin (2.3 mg/kg/day); AIBP (compound 7) (0.5 μg/5 μl); saline (5 μl). B, Baseline (day 0 in A) withdrawal thresholds before the start of cisplatin intervention. Mean±S.E.M. (n=15 for vehicle treated and n=16 for TAM treated AIBP-imKO mice and n=8 for littermates Apoa1bp^{fl/fl} no-Cre [WT] mice treated with TAM). C, WT and Cx3cr1-Cre_{ERT2} (no floxed genes) mice were tested for withdrawal threshold before (naïve, day −7 in panel A timeline) and after (TAM, day 0) tamoxifen injection regimen (10 mg/mL, 200 μL/day for 5 days). (n=5 per group. One animal was found dead for WT+TAM group). No statistical differences were found. D-F, Withdrawal thresholds following i.p. cisplatin and i.t. saline or AIBP (compound 7) injections in TAM-induced AIBP-imKO mice (C; n=6-7, data from 2 independent experiments), non-induced (vehicle) AIBP-imKO mice (D; n=4-5, data from 2 independent experiments), and bred in-house whole body AIBP knockout mice (E; n=4 per group). G, Withdrawal thresholds in WT and tamoxifen-induced TLR4-imKO mice following cisplatin injections (n=4 for bred inhouse wild type and n=7 for TLR4-imKO mice). Mean±S.E.M. *, P<0.05;**, P<0.01. Two-way ANOVA with Bonferroni post-hoc test for multiple comparisons in grouped analyses; one-way ANOVA with Tukey post-hoc test for multiple comparisons of >2 groups.

FIG. 9: Identifies sequence motifs that contribute to AIBP binding to TLR4: FIG. 9. Identification of the domain in the AIBP molecule responsible for TLR4 binding. A, Human AIBP: signal peptide (aa 1-24), previously uncharacterized N-terminal domain (aa 25-51), and YjeF_N domain (aa 52-288). B, Flag-tagged deletion mutants of human AIBP were co-expressed in HEK293 cells with the Flag-tagged TLR4 ectodomain (eTLR4). Cell lysates were immunoprecipitated (IP) with an anti-TLR4 antibody and immunoblotted (IB) with an anti-Flag antibody. C, His-tagged human (hu), mouse (mo) and zebrafish (zf) AIBP, all lacking the signal peptide, expressed in a baculovirus/insect cell system, were combined in a test-tube with eTLR4-His, followed by IP with an anti-TLR4 antibody and IB with an anti-His antibody. D-H, Binding of His-tagged wild type (wt, 25-288 aa) and the deletion mutant (mut, 52-288 aa) human AIBP to eTLR4, APOA1 and microglia. IP of eTLR4 and wtAIBP or mutAIBP in a test tube with an anti-AIBP antibody; blot and quantification from 3 independent experiments (D). ELISA with plates coated with eTLR4 and incubated with wtAIBP or mutAIBP (n=3) (E). ELISA with plates coated with BSA, wtAIBP or mutAIBP and incubated with APOA1 (F). Flow cytometry (n=6) (F) and confocal imaging (G) showing binding of wtAIBP and mutAIBP (2 μg/mL) to BV-2 microglia cells, unstimulated or treated for 15 min with LPS (100 ng/mL). Detection with an anti-His antibody (flow) and an anti-TLR4 antibody (imaging). Scale bar, 10 μm. Mean±S.E.M. ***, P<0.001;**, P<0.01;*, P<0.05; n.s., non-significant. Two-way ANOVA with Bonferroni post-hoc test for multiple comparisons in time-course analysis; t-test for 2 groups; and one-way ANOVA with Tukey post-hoc test for multiple comparisons of more than 2 groups.

FIG. 10 demonstrates that a mutant AIBP that does not bind TLR4 does not reverse pain behavior in CIPN mice and suggests a model for AIBP-modulation of TLR-mediated pain:

FIG. 10. Intrathecal delivery of AIBP lacking the TLR4 binding domain cannot alleviate CIPN allodynia. A-B, TLR4 dimerization (A) and lipid rafts (B) in BV-2 cells pre-treated with wtAIBP or mutAIBP (0.2 μg/mL) and stimulated with 100 ng/ml LPS for 15 min. Mean±S.E.M. (n=7 for control group, n=5 for mutAIBP group, n=9 for LPS group and n=8 for wtAIBP group in TLR4 dimerization analysis; n=8 for control group and n=13 for mutAIBP, LPS and wtAIBP group in lipid rafts analysis; data from 2 independent experiments). C, Withdrawal thresholds in WT mice that received i.t. AIBP (0.5 μg/5 μL) or saline (5 μL), followed by i.t. LPS (0.1 μg/5 μL); n=5 per group. D, Withdrawal thresholds in WT mice in response to i.p. cisplatin (2.3 mg/kg/day), followed by i.t. wtAIBP (0.5 μg/5 μL), mutAIBP (0.5 μg/5 μL) or saline (5 μL). Naïve mice did not receive any injections (n=7 for naive group, n=8 for wtAIBP and mutAIBP group, n=9 for i.t. saline group; data from 2 independent experiments). E-F, TLR4 dimerization (E) and lipid rafts (F) in CD11b⁺/TMEM119⁺ microglia from lumbar spinal cord of mice in experimental groups shown in panel D, at day 21 (n=7-9; data from 2 independent experiments). *, P<0.05; **P<0.01; ***, P<0.005. Two-way ANOVA with Bonferroni post-hoc test for multiple comparisons in time-course analysis; and one-way ANOVA with Tukey post-hoc test for multiple comparisons of >2 groups. G, Diagram illustrating the effect of CIPN and AIBP (compound 7) treatment on microglia gene expression and lipid droplet accumulation. Black dots in the plasma membrane and the ER depict cholesterol.

FIG. 11 postulates a model for exposure of the TLR4 binding site of AIBP in a modified AIBP sequence: FIG. 11. Model of unfolding or exposing a cryptic N-terminal domain in the AIBP molecule. The diagram summarizes and illustrates results of experiments shown in FIGS. 12-14, which demonstrate that in native AIBP the N-terminal domain (green) is hidden or cryptic or not sufficiently exposed to mediate AIBP binding to TLR4 (top panel). Extending the N-terminus with additional amino acids (orange) changes the AIBP conformation and makes the N-terminal domain of AIBP (green) accessible for TLR4 binding (bottom panel). FIG. 12. One example of the amino acid sequence of an exemplary engineered AIBP as provided herein. The amino acid sequence of an extended AIBP molecule depicted in the bottom panel of FIG. 11. Blue letters, amino acids from the native AIBP sequence; green box, the TLR4-binding sequence (amino acids 25-51 of the human AIBP sequence); black letters and red box, added amino acids. FIG. 13 demonstrates TLR4 binding of certain modified AIBP sequences derived from a baculovirus expression system:

FIG. 13. TLR4 binding of various engineered forms of AIBP. All proteins were expressed and purified from a baculovirus/insect cell system. The top drawing for His-d24AIBP corresponds to the amino acid sequence shown in FIG. 12. The amino acid sequence below the top drawing shows the sequence of the orange box “cleavable His tag.” All other drawings show different modifications and corresponding changes in the amino acid sequence introduced to the AIBP molecule. The green “N-terminal domain” box depicts the amino acid 25-51 sequence of native AIBP. The column on the right shows the results of co-immunoprecipitation experiments of the AIBP variants with a recombinant ectodomain of TLR4. FIG. 14 demonstrates TLR4 binding of certain modified AIBP sequences from a mammalian expression system:

FIG. 14. TLR4 binding of various engineered forms of AIBP, continued 1. All proteins were co-expressed with the full-length TLR4 in a mammalian system. SS, secretion signal, corresponding to the amino acids 1-24 in the human AIBP sequence. The column on the right shows the results of co-immunoprecipitation from cell lysates of the AIBP variants with TLR4.

FIG. 15. Various AIBP constructs to optimize the structure for TLR4 affinity: baculovirus/insect cell expression system.

FIG. 16 confirms that N-terminal modification to AIBP is necessary for TLR4 binding in an E. coli expression system:

FIG. 16. TLR4 binding of various engineered forms of AIBP, continued 2. All proteins were expressed and purified from an E. coli. The column on the right shows the results of co-immunoprecipitation experiments of the AIBP variants with a recombinant ectodomain of TLR4, there was no TLR4 binding using the AIBP variants d24 AIBP-his or d51 AIBP-his.

FIG. 17A-D (or, FIG. S1, or supplementary FIG. 1) provides validation of the specificity of TLR4 antibodies used for flow cytometry and microscopy, and also shows TLR4 dimerization and lipid rafts measured in dorsal root ganglia macrophages:

FIG. 17A graphically illustrates flow cytometry of single cell suspensions from spinal cords of WT (left images) and Tlr4−/− mice (right images) showing TLR4-APC and TLR4/MD2-PE antibodies staining of CD11b+(PercP-Cy5.5)/TMEM199+(Pe-Cy7) microglia;

FIG. 17B illustrates confocal images of peritoneal elicited macrophages from WT and Tlr4−/− mice co-stained with F4/80-FITC and TLR4-647 antibodies; Scale bar, 5 μm; and

FIG. 17C-D graphically illustrate flow cytometry analysis of CD11b+ DRG macrophages cells showing TLR4 dimerization (FIG. 17C) and lipid raft content measured by CTxB staining (FIG. 17D) 24 hours after i.t. saline or AIBP, i.e. at day 8 of the time course shown in FIG. 17A; data from 2 independent experiments (n=5 for control and AIBP group and n=9 for cisplatin i.t. saline group).

FIG. 18A-E (or, FIG. S2, or supplementary FIG. 2) shows FACS sorting strategy for spinal microglia, quality controls and phenotypic controls for RNA-seq:

FIG. 18A illustrates sorting strategy for lumbar CD11b+TMEM119+ spinal microglia, including: SSC-A and FSC-A, SSC-W and SSC-H, UVE/DEAD (APC-Cy7-A) and SSC-A, GLAST1 and CD24, and, CD11b and TMEM119;

FIG. 18B illustrates flow cytometry analysis of sorted microglia measuring purity of sorted cells and absence of GLAST1+ astrocytes or CD24+ neurons, including TMEM119 and CD11b, SSC-A and GLAST1, and SSC-1 and CD24;

FIG. 18C illustrates microglial linage analysis with a heatmap of microglia specific genes. Log+1 of normalized counts from all samples was calculated for the 40 microglia specific genes listed in Butovsky et. al, 2014, as well as for the 3 genes that are expressed at low levels in microglia but at high levels specifically in neurons (Nefl), oligodendrocytes (Omg) or astrocytes (Slc6a1);

FIG. 18D-E illustrate heatmaps of CIPN-repressed genes that were up-regulated by AIBP (group 4) (FIG. 18D) and CIPN-induced genes that were downregulated by AIBP (group 3) in wildtype mice (FIG. 18E); Log 2 normalized gene counts scaled by row and columns represent all technical replicates of the 3 biological samples.

FIG. 19A-D (or, FIG. S3, or supplementary FIG. 3) provide immunohistochemical validation of conditional knockout of ABCA1 and ABCG1 in spinal microglia of tamoxifen-induced ABC-imKO mice:

IHC of spinal cord frozen sections from vehicle and tamoxifen induced ABC-imKO mice, showing colocalization of ABCA1 and ABCG1 staining with IBA1 (microglia), NeuN (neurons) and GFAP (astrocytes). Slides were mounted with Prolog Gold with DAPI. Confocal images were acquired with a 63× objective and analyzed with ImageJ software for colocalization. Colocalization masks and Pearson's R-values, Manders' colocalization coefficients above threshold and randomization Costes P values were calculated as described in Methods for at least 1 slide for each animal in the experiment. Representative images and values shown correspond to one animal per condition. Scale bar, 50 μm.

FIG. 20A-E (or, FIG. S4, or supplementary FIG. 4) shows tactile allodynia data for tamoxifen-treated WT mice in i.t. LPS and CIPN experiments. It also provides additional RNA-seq data for ABC-imKO dependent genes and the cisplatin effect on ABC-imKO vs. WT mice. As a control for ABC-imKO mice, inhouse bred WT littermate mice were subjected to the tamoxifen regimen (TAM, 200 μL/day, 10 mg/mL, 5 consecutive days), followed by (FIG. 20A) i.t. injection of AIBP (0.5 μg/5 μL) or saline (5 μL) and i.t. LPS (0.1 μg/5 μL) 2 hours later (n=4 for i.t saline and n=5 for i.t. AIBP); and (FIG. 20B) i.p. injections of cisplatin (2.3 mg/Kg) on day 1 and day 3 followed by i.t. injection of AIBP (0.5 μg/5 μL) or saline (5 μL) on day 7 (n=4 per group). Tactile allodynia (withdrawal thresholds) was measured using von Frey filaments. Mean±S.E.M. *, P<0.05. Two-way ANOVA with Bonferroni post hoc test for multiple comparisons in time-course analysis. C, ABC-imKO mice were injected with TAM and then cisplatin as above, followed by i.t. saline (5 μL), AIBP (0.5 μg/5 μL) or hp-β-CD (0.25 mg/5 μL) on day 7. Shown are tactile thresholds 24 h after the i.t. injection. Mean±S.E.M. (n=3-4 per group). **, P<0.01. One-way ANOVA with Dunnett's multiple comparisons test. FIG. 20D, Heatmap of differentially regulated genes across all conditions (naïve, induced by cisplatin/saline or cisplatin/AIBP) regulated in an ABC-imKO manner. All significant genes from likelihood ratio test using a reduced model without interaction term (condition: genotype). Log 2 normalized gene counts scaled by row and columns represent all technical replicates of the 2-3 biological samples from each group. FIG. 20E, Heatmap of pathway enrichment of cisplatin upregulated genes in WT and ABC-imKO microglia using cutoff P<0.05, enrichment>1.5 and a minimum overlap of 3 genes in the pathway. Heatmap depicts common and specific pathways enriched by cisplatin in both genotypes.

FIG. 21 (or, FIG. S5, or supplementary FIG. 5) provides immunohistochemical validation of AIBP knockout in spinal microglia of tamoxifen-induced AIBP-imKO mice. It also demonstrates that the BE-1 monoclonal antibody has similar affinity to wtAIBP and mutAIBP. FIG. 21A: IHC of spinal cord frozen sections from vehicle and tamoxifen induced AIBP-imKO mice, showing colocalization of AIBP staining with IBA1 (microglia), NeuN (neurons) and GFAP (astrocytes). Slides were mounted with Prolog Gold with DAPI. Confocal images were acquired with a 63× objective and analyzed with ImageJ software for colocalization. Colocalization masks and Pearson's R-values, Manders' colocalization coefficients and randomization Costes' P values were calculated as described in Methods for at least 1 slide for each animal in the experiment. Representative images and values shown correspond to one animal per condition. Scale bar, 50 μm. FIG. 21B: Sandwich ELISA using BE-1 as a capture antibody in a microtiter plate. Dose response curves to wtAIBP and mutAIBP were detected using a rabbit polyclonal anti-AIBP antibody. No statistical differences were found for BE-1 affinity to wtAIBP and mutAIBP using two-way ANOVA with Bonferroni post hoc test for multiple comparisons.

### Example 2. Structural Determinants of AIBP Binding to TLR4

This Example summarizes results of pull-down experiments to test binding of different AIBP variants expressed in insect, mammalian or bacterial systems to the ectodomain of TLR4. Results for this example are unexpected in that the depictions for activity in FIGS. 13 and 14 demonstrate that not all N-terminal modifications expose the TLR4 binding domain. By example, the putative cleavage products of N-terminal His-tags containing a cleavage site do not demonstrate TLR4 binding. These unexpected data suggest that N-terminal modifications to AIBP polypeptides have specific amino acid composition requirements.

A pulldown assay was performed using compounds as provided herein. Compounds 3, 7, 8 or 9 and other constructs described in FIGS. 13 and 14 were purified from either a baculovirus (BD Bioscience) or CHO (ExpiCHO, Expression Systems, ThermoFisher) cell expression system and incubated with TLR4 protein (Sino biological). Pull-down was performed with anti-AIBP antibody described. Bound TLR4 to AIBP was detected by western blot with an anti-his antibody (both modified AIBP and TLR4 have his-tag). Detailed experimental information is provided in example 1 as to pull down method.

An alternate assay used transfection of modified AIBP and TLR4 into HEK293 cells. For this study, transfected Flag-AIBP (exemplified for compounds 5 and 6) and Flag-TLR4-his constructs were expressed, transfected cells harvested, and lysed. Cell lysates were co-immunoprecipitated with anti-TLR4 antibody then immunoblotted with an anti-flag antibody. Detailed experimental information is provided in example 1 as to pull down method.

### Example 3: Efficacy Demonstrated in Exemplary Models: Asthma

### Reduced AIBP Expression in Bronchial Epithelial Cells of Asthmatic Patients:

Apolipoprotein A-I binding protein (AIBP; gene name APOA1BP or NAXE) is a secreted protein (1), which facilitates removal of excess cholesterol from activated cells, including primary alveolar macrophages, endothelial cells, and microglia (2-4). We have demonstrated that the pulmonary surfactant can serve as a cholesterol acceptor when incubated with alveolar macrophages (4). In addition, ApoA-I is found in bronchoalveolar lavage fluid (BALF) (5). These findings suggests that cholesterol efflux occurs not only in blood and tissues, but also in the pulmonary airspace. AIBP binds to surfactant protein B and augments cholesterol efflux from alveolar macrophages to surfactant (4). This results in normalization of lipid raft content in the plasma membrane, reduced inflammatory signaling and reduced expression of inflammatory cytokines in alveolar macrophages. In response to inhaled LPS lung injury, AIBP is secreted into BALF (4). In addition, AIBP facilitates mitophagy, helps maintain mitochondrial function and reduces oxidative stress in macrophages (6). The hypothesis that AIBP expression serves to protect against inflammation implies that raising AIBP levels in the lung may have a therapeutic effect.

Because of the broad anti-inflammatory protections afforded by AIBP in the lung (4) and other tissues (3, 7), in this work we examined whether lung expression of endogenous AIBP is affected in asthma patients and if inhaled AIBP can reduce pulmonary inflammation and alleviate airway hyperresponsiveness in a mouse model of asthma.

Immunohistochemistry of postmortem human lung tissue obtained from non-asthmatic subjects revealed a pattern of predominant AIBP protein expression in bronchial epithelial cells. Interestingly, the AIBP expression was significantly reduced in the bronchial epithelial cells of postmortem lungs from subjects with asthma (see FIG. 22A). In addition, primary bronchial epithelial cells isolated from postmortem lungs of subjects with asthma had a significantly lower APOA1BP mRNA expression compared to that in non-asthmatic subjects (see FIG. 21D). Similar to human asthma, expression of endogenous AIBP in bronchial epithelium was significantly reduced in house dust mite (HDM)-challenged mice, compared to control mice that received intranasal PBS (see FIG. 22C). This pattern of reduced AIBP expression in bronchial epithelium following acute HDM challenge in mice was not observed in mouse acute lung injury model (4). In addition, the lung cell types expressing the highest levels of AIBP differed in the two models, with the highest AIBP expression in acute lung injury observed in recruited inflammatory cells (i.e., neutrophils and macrophages) (4). In contrast, the predominant recruited inflammatory cell following acute HDM challenge (i.e., eosinophils) did not express high levels of AIBP.

Because endogenous AIBP expression was reduced in asthma (FIG. 21) and administration of AIBP, either as a recombinant protein or via adeno-associated virus delivery, produced anti-inflammatory and protective effects in neuroinflammation and neuropathic pain (7), vascular inflammation and atherosclerosis (3), and acute lung injury (4), we tested whether intranasal delivery of a recombinant AIBP (Compound 7) will have a therapeutic effect in a mouse model of asthma.

Compound 7 was administered 2 hours before the administration of HDM.

Four weekly administrations of intranasal HDM in female mice induce lung eosinophilic inflammation and the airway hyperresponsiveness (AHR) to methacholine challenge (8). Two doses of Compound 7, 2.5 and 25 μg, or vehicle (PBS) were administered to 8-week-old C57BL/6J female and male mice weekly, via intranasal instillation, 2 hours before the intranasal HDM. Intranasal Compound 7 produced no apparent adverse effects. As expected, HDM-challenged female mice pre-treated with PBS developed AHR. In contrast, Compound 7 pre-treatment reduced, in a dose-dependent manner, HDM-induced AHR, with the 25-μg dose resulting in nearly complete inhibition of AHR (FIG. 23A) and also induced dose-dependent reductions in HDM-induced lung and BAL eosinophilia (FIG. 23B-C). In this model, HDM induces no noticeable changes in the numbers of alveolar macrophages or neutrophils (8). Similar results were obtained for Compound 7 in male mice (FIG. 23D-F).

Taken together, our studies demonstrate significantly reduced AIBP expression in human bronchial epithelial cells in asthmatics compared to non-asthmatics, as well as in bronchial epithelial cells following HDM challenge in a mouse model of asthma. This results correspond to findings of reduced ApoA-I levels in BALF of asthmatics compared to non-asthmatics (5). Because airway epithelial and bronchial inflammation is a major component of asthma leading to airway smooth muscle contraction, airways obstruction and asthma exacerbations (9), restoring levels of AIBP, which has anti-inflammatory properties, may present a novel therapeutic strategy for asthma. Our results with intranasal administration of Compound 7, showing a therapeutic effect in the acute HDM mouse model of asthma, support this proposition. As inhaled corticosteroids (ICS) are the cornerstone of treatment for moderate/severe asthma, further studies in pre-clinical models and subsequently in human subjects with asthma are needed to determine whether Compound 7 has an additive anti-inflammatory effect on asthma control when combined with ICS, and/or be an other anti-inflammatory to ICS in asthma subjects who do not respond well to or have side effects from ICS.

### Materials and Methods for Example 3

### Human Lung Specimens

Postmortem human lungs from asthmatics and non-asthmatics were procured by the Arkansas Regional Organ Recovery Agency and by the National Disease Research Interchange and delivered to the Lung Cell Biology Laboratory at the Arkansas Children's Research Institute. Immunohistochemistry was conducted at UC San Diego. Subjects were categorized as asthmatic if they had a physician diagnosis of asthma listed in the hospital medical record and used asthma medications at the time of death. Subjects were categorized as non-asthmatic if they had no physician diagnosis of asthma as well as no asthma medication use listed in the hospital medical record at the time of death. The acquisition of deceased donor tissue was reviewed by the University of Arkansas for Medical Sciences Institutional Review Board and determined not to be human subject research. This study was approved by the University of California, San Diego Human Research Protections program.

### Human Bronchial Epithelial Cells

Primary bronchial epithelial cells were isolated from bronchi of postmortem lungs. In brief, bronchi were dissected, and the interior of each bronchus was scraped with a Cell Lifter (Corning, Inc.) to obtain bronchial epithelial cells. The bronchial epithelial cells were collected and cultured in CnT-17 media (Cellntec, Bern, Switzerland). These primary bronchial epithelial cells were of >95% pure as assessed by E-cadherin expression by flow cytometry.

### Human and Mouse Lung Immunohistochemistry

Paraffin-embedded lung sections were stained using a cocktail of mouse anti-human and anti-mouse AIBP monoclonal antibodies A7 and BE-1 developed in our lab (6, 7) and mixed at 1:2 ratio. Due to close homology of mouse and human AIBP, both antibodies recognize the mouse and the human protein. Quantification of AIBP-positive staining in epithelial cells was performed for each lung section using an image analysis system (Image-Pro plus, Media Cybernetics), and results were expressed as AIBP-positive area of bronchial epithelium per μm length of the bronchial basal membrane in human specimens. AIBP expression in the mouse lung was measured using a mean grey value tool in Image J (NIH), and the values in the cytosol of bronchial epithelium of bronchiole with a 150-200 μm internal diameter were normalized to that in adjacent alveolae. The operators were blinded to the identity of samples.

APOA1BP mRNA Quantification

To quantify APOA1BP mRNA in human bronchial epithelial cells from asthmatics and non-asthmatics, total RNA from each cell sample was processed for RT-qPCR as previously described (8). In brief, samples were treated with RNA-STAT-60 (TelTest), and reverse-transcribed with Oligo-dT and SuperScript II kit (Life Technologies). qPCR was performed with TaqMan PCR Master Mix and TaqMan primers for human APOA1BP (Hs.PT.58.22278956, Integrated DNA Technologies, Coralville, IA). The relative amounts of APOA1BP mRNA were normalized to those of the housekeeping gene hypoxanthine phosphoribosyltransferase-1 (HPRT1).

### Production of Compound 7

In brief, Compound 7 was expressed in a baculovirus/insect cell system to ensure posttranslational modification and endotoxin-free preparation and purified by affinity chromatography using a Ni-NTA agarose column, followed by ion exchange chromatography and buffer replacement. The product was greater than 90% pure, with no detectable aggregates (HPLC-SEC) and residual endotoxin less than 0.2 EU/mg. Storage-stability study of Compound 7 for up to 6 months at −80° C. or for 1 week at 4° C. did not show any loss of its titer or purity.

### Acute HDM Mouse Model of Asthma

All experiments were conducted according to protocols approved by the Institutional Animal Care and Use Committee (IACUC) of the University of California, San Diego. Wild type C57BL/6J mice (male and female) aged 8 weeks were administered 100 μg of intranasal HDM (Dermatophagoides pteronyssinus) extract (Greer Laboratories) on days 0, 7, 14, and 21 as previously described (8). Two hours prior to each HDM administration, 50 μl of PBS control or Compound 7 solution, either 2.5 μg or 25 μg dose, were given intranasally. Control group received intranasal PBS instead of HDM. On day 24, airway hyperresponsiveness to methacholine was assessed as described (8), and the mice were sacrificed to collect the BAL and the lungs. BAL was collected by lavage of 1 ml PBS via tracheal catheter, centrifuged and the pellet was resuspended in 1 ml PBS. After determining BAL total cell count, differential cell counts were quantified in Wright-Giemsa stained slides (8). Lung eosinophil counts were quantified in the peribronchial space in lung paraffin-embedded sections stained with an anti-mouse major basic protein (MBP) rabbit polyclonal antibody (kindly provided by Mayo Foundation for Medical Education and Research). Results are expressed as the number of peribronchial cells staining positive per bronchiole with a 150-200 μm internal diameter. At least 5 bronchioles were counted in each slide. The operator was blinded to the identity of samples.

Statistics: All results are presented as Mean±SEM. A statistical software package (GraphPad Prism) was used for the analysis. Mann-Whitney test was used for analysis of 2 groups. Two-way or one-way ANOVA with post hoc Tukey's multiple comparisons test was used when more than 2 groups were compared. P-values of less than 0.05 were considered statistically significant.

### Figure Legends for Example 3

FIG. 22. Reduced AIBP expression in bronchial epithelium. A, Postmortem lung specimens from human subjects without asthma and with asthma stained with anti-AIBP antibodies. Quantification of AIBP-positive bronchial epithelium (n=6). B, APOA1BP mRNA in bronchial epithelial cells isolated from non-asthmatics (n=9) and asthmatics (n=11) normalized to HPRT1. C, Lungs from mice that received 4 weekly intranasal doses of vehicle or 100 μg HDM stained with anti-AIBP antibodies. Quantification of AIBP staining in bronchial epithelium (n=8). Mean±SEM;*, P<0.05;***, P<0.001.

FIG. 23. RFT1081 reduces airway hyperresponsiveness and eosinophilic pulmonary inflammation in an acute HDM model of asthma in female and male mice. Female (A-C) and male (D-F) C57BL/6J mice received 4 weekly intranasal instillations of 2.5 μg or 25.0 μg of RFT1081 or PBS. Two hours later, the mice received intranasal instillations of 100 μg HDM or vehicle. Three days after last challenge, mice were tested for airway resistance to methacholine (A, D), and lung (B, E) and BAL (C, F) were collected for analyses as indicated. Mean±SEM; n=8 mice per group. *, P<0.05;**, P<0.01;****, P<0.001;***, P<0.0001

### Example 4: Efficacy Demonstrated in Exemplary Methods: Glaucoma

AAV-AIBP protects retinal ganglion cells and their axons and improves visual function in experimental glaucoma:

Glaucomatous DBA/2J (D2) mouse model. The advantage of using the genetic D2 model, together with age-matched non-glaucomatous control D2-Gpnmb⁺ mice, is that it replicates the chronic IOP elevation of human glaucoma, with retinal pathology developing with age, at around 9-10 months (1, 2). We do realize that as any animal model, D2 has its limitations as the glaucoma-like pathology develops in these mice secondary to anterior segment anomalies with synechiae and pigment dispersion (1, 2). In preliminary studies, we observed significantly elevated cholesterol content in the retina of Apoa1bp^{-/-} compared with WT mice (see FIG. 24A), suggesting that AIBP deficiency induces excessive cholesterol accumulation in the retina. Of note, in 10-mo-old glaucomatous D2 mice, we discovered that cholesterol content was also significantly elevated in the retina compared with D2-Gpnmb⁺ mice (FIG. 24B). Thus, we tested if overexpression of AIBP by in vivo delivery of AAV-AIBP can reverse excessive cholesterol accumulation, protect RGCs and their axons, and preserve central visual pathway in glaucomatous D2 mice. The AAV serotype we used in mouse model was AAV-DJ/8, and it expressed mouse AIBP in which a fibronectin signal peptide ensured robust protein secretion. We intravitreally injected AAV-Null or AAV-AIBP at the age of 5 months and analyzed tissue samples (retina, optic nerve head and brain) at the age of 10 months. We assessed RGC and its axon survival by staining for RNA-binding protein with multiple splicing (RBPMS) and neurofilament 68 (NF68) and the preservation of central visual pathway by cholera toxin subunit B (CTB) labeling of superior colliculus (SC), which shows the entire retinal projection via active uptake and transport^{3,4}. Following AAV-AIBP injection, AIBP protein expression was detected in the retina at age of 10 months (FIG. 24E). The AAV-AIBP, but not AAV-Null, significantly reduced cholesterol content (FIGS. 24C and D), protected RGCs in the middle and peripheral areas of glaucomatous D2 retina (FIGS. 24G and H). In addition, we observe a significant improvement in CTB transport to the SC (FIG. 24J-M), suggesting that AAV-AIBP helped preserve structural and functional integrity of the optic nerve.

Microbead-induced ocular hypertension model. Recently, we successfully developed a mouse model of microbead-induced ocular hypertension, which showed a significant loss of RGCs at 6 weeks post procedure in 4-mo-old C57BL/6J mice (FIG. 25). To further validate the protective effects of AAV-AIBP on RGCs and visual function in vivo, we intravitreally injected AAV-Null or AAV-AIBP 3 weeks before the microbead injection. AAV-AIBP significantly reduced RGC death (see FIG. 25C) and importantly, ameliorated visual dysfunction (see FIG. 25D).

Optic nerve crush (ONC) model. ONC serves as a useful model not only of traumatic optic neuropathy but also of glaucomatous injury, as it similarly induces RGC death and degeneration⁵. We intravitreally injected AAV-Null or AAV-AIBP 3 weeks before ONC and then assessed RGC survival by RBPMS staining at 1 week after ONC. It was found that overexpression of AIBP protected RGCs against ONC injury (FIG. 26).

Collectively, those findings demonstrate that in three different in vivo models of glaucomatous neurodegeneration, AAV-delivered AIBP expression reduces cholesterol content, protects RGC and their axons, inhibits microglial activation (not shown), and preserves visual function.

### Figure Legends for Example 4

FIG. 24. AAV-AIBP reduces retinal neurodegeneration in glaucomatous DBA/2J (D2) mice. A and B, Apoa1bp^{-/-} mice. Filipin staining for cholesterol (A). Quantification of filipin intensity in the inner retina (B). C-M, Glaucomatous D2 mice. Filipin staining for cholesterol (C). Quantification of filipin intensity in the inner retina (D). Confirmation of AIBP expression in the retina by immunoblot with anti-His antibody (E). IOP measurements (F). RBPMS (green)-positive RGCs in the peripheral area of the retina (G). Quantitative analysis of RGC survival in the middle and peripheral retinas (H). NF68 (green)-positive axons in the glial lamina (I). CTB labeling (red) and Brn3a (green) in the retina (J). CTB labeling in the SC (K and L). Quantification of CTB intensity in the SC (M). Mean±SEM; n=5-8 retinas. *P<0.05, **P<0.01, ***P<0.001, and ****P<0.0001 (One-way ANOVA, Turkey's multiple comparisons test). Scale bars: 20 μm (A and C) and 50 μm (G and I).

FIG. 25. AAV-AIBP reduces retinal neurodegeneration and improves visual function in a microbead-induced hypertension mouse model. A, IOP time course in microbead-injected eyes. B, Representative images from the peripheral area of the retina by TUJI staining at 6 weeks after microbead injection. C, Quantitative analysis of RGC survival in the middle area of the retina. D, Visual function measurement by PERG analysis. Mean±SM; n=5-8 retinas. *P<0.05, ***P<0.001 and ****P<0.0001 (One-way ANOVA, Turkey's multiple comparisons test). Bar: 50 μm.

FIG. 26. AAV-AIBP reduces retinal neurodegeneration and a mouse optic nerve crash model. A, Representative images for RBPMS-positive RGCs in the middle area of the retina following ONC injury; B, Quantitative analysis of RGC survival in the middle and peripheral areas of the retina. Mean±SEM; n=5-8 retinas. *P<0.05 **P<0.01, ****P<0.0001 (One-way ANOVA, Turkey's multiple comparisons test). Bar: 50 μm.

### REFERENCES EXAMPLE 1

Ahn, J. H., et al. (2019). Meningeal lymphatic vessels at the skull base drain cerebrospinal fluid. Nature 572, 62-66.
Akashi, S., et al. (2003). Lipopolysaccharide interaction with cell surface Toll-like receptor 4-MD-2: higher affinity than that with MD-2 or CD14. J Exp Med 198, 1035-1042.
Blasi, E., et al., (1990). Immortalization of murine microglial cells by a v-raf/v-myc carrying retrovirus. J Neuroimmunol 27, 229-237.
Brandolini, L., et al., (2019). Chemokine Signaling in Chemotherapy-Induced Neuropathic Pain. Int J Mol Sci 20, 2904.
Bruno, K., et al. (2018). Targeting toll-like receptor-4 (TLR4)-an emerging therapeutic target for persistent pain states. Pain 159, 1908-1915.
Chaplan, S. R., et al. (1994). Quantitative assessment of tactile allodynia in the rat paw. J Neurosci Methods 53, 55-63.
Chen, T., et al. (2017). Interactions of Notch1 and TLR4 signaling pathways in DRG neurons of in vivo and in vitro models of diabetic neuropathy. Sci Rep 7, 14923.
Cheng, A. M., et al., (2012). Apolipoprotein A-I attenuates palmitate-mediated NF-kappaB activation by reducing Toll-like receptor-4 recruitment into lipid rafts. PLOS One 7, e33917.
Choi, S.-H., et al., (2020). Intracellular AIBP (Apolipoprotein A-I Binding Protein) Regulates Oxidized LDL (Low-Density Lipoprotein)-Induced Mitophagy in Macrophages. Arterioscler Thromb Vasc Biol 0, ATVBAHA. 120.315485.
Choi, S. H., et al. (2018). AIBP augments cholesterol efflux from alveolar macrophages to surfactant and reduces acute lung inflammation. JCI Insight 3, e120519.
Dobin, A., et al., (2013). STAR: ultrafast universal RNA-seq aligner. Bioinformatics 29, 15-21.
Ewels, P., et al. (2016). MultiQC: summarize analysis results for multiple tools and samples in a single report. Bioinformatics 32, 3047-3048.
Fang, L., et al. (2013). Control of angiogenesis by AIBP-mediated cholesterol efflux. Nature 498, 118-122.
Furfari, A., et al. (2017). Genetic biomarkers associated with changes in quality of life and pain following palliative radiotherapy in patients with bone metastases. Ann Palliat Med 6, S248-S256.
Gay A., R. D., and Arun Radhakrishnan (2015). Switch-like Responses of Two Cholesterol Sensors Do Not Require Protein Oligomerization in Membranes. Biophysical Journal 108, 1459-1469.
Gregus, A. M., et al. (2018). Inhibition of spinal 15-LOX-1 attenuates TLR4-dependent, nonsteroidal anti-inflammatory drug-unresponsive hyperalgesia in male rats. Pain 159, 2620-2629.
Gu, Q., et al. (2019). AIBP-mediated cholesterol efflux instructs hematopoietic stem and progenitor cell fate. Science 363, 1085-1088.
Hartley, S. W., and Mullikin, J. C. (2015). QoRTs: a comprehensive toolset for quality control and data processing of RNA-Seq experiments. BMC Bioinformatics 16, 224.
He, C., et al., (2017). High-resolution imaging and quantification of plasma membrane cholesterol by NanoSIMS. Proc Natl Acad Sci USA 114, 2000-2005.
Hossaini, M., et al. (2010). Nociceptive stimulation induces expression of Arc/Arg3.1 in the spinal cord with a preference for neurons containing enkephalin. Mol Pain 6, 43.
Hu, L. Y., et al. (2018). Triggering receptor expressed on myeloid cells 2 (TREM2) dependent microglial activation promotes cisplatin-induced peripheral neuropathy in mice. Brain Behav Immun 68, 132-145.
Hylden, J. L., and Wilcox, G. L. (1980). Intrathecal morphine in mice: a new technique. Eur J Pharmacol 67, 313-316.
Jaitin, D. A., et al. (2019). Lipid-Associated Macrophages Control Metabolic Homeostasis in a Trem2-Dependent Manner. Cell 178, 686-698 e614.
Jha, K. N., et al. (2008). Biochemical and structural characterization of apolipoprotein A-I binding protein, a novel phosphoprotein with a potential role in sperm capacitation. Endocrinology 149, 2108-2120.
Karasinska, J. M., et al. (2013). ABCA1 influences neuroinflammation and neuronal death. Neurobiol Dis 54, 445-455.
Kennedy, H. S., et al. (2013). Comparison of standard laminectomy with an optimized ejection method for the removal of spinal cords from rats and mice. J Histotechnol 36, 86-91.
Lees, J. G., et al. (2017). Immune-mediated processes implicated in chemotherapy-induced peripheral neuropathy. Eur J Cancer 73, 22-29.
Li, Y., et al. (2021). Chemotherapy-induced peripheral neuropathy in a dish: dorsal root ganglion cells treated in vitro with paclitaxel show biochemical and physiological responses parallel to that seen in vivo. Pain 162, 84-96.
Liu, L., and Duff, K. (2008). A technique for serial collection of cerebrospinal fluid from the cisterna magna in mouse. Journal of Visualized Experiments 21, 960.
Love, M. I., et al. (2014). Moderated estimation of fold change and dispersion for RNA-seq data with DESeq2. Genome Biol 15, 550.
Makker, P. G., et al. (2017). Characterisation of Immune and Neuroinflammatory Changes Associated with Chemotherapy-Induced Peripheral Neuropathy. PLOS One 12, e0170814.
Marschallinger, J. et al. (2020). Lipid-droplet-accumulating microglia represent a dysfunctional and proinflammatory state in the aging brain. Nat Neurosci 23, 194-208.
Masuda, T., et al. (2019). Spatial and temporal heterogeneity of mouse and human microglia at single-cell resolution. Nature 566, 388-392.
Miller, Y. I., et al. (2020). Lipid rafts in glial cells: role in neuroinflammation and pain processing. J Lipid Res 61, 655-666.
Nugent, A. A., et al. (2020). TREM2 Regulates Microglial Cholesterol Metabolism upon Chronic Phagocytic Challenge. Neuron 105, 837-854 e839.
Oliveira, A., et al. (2014). Interleukin-1beta genotype and circulating levels in cancer patients: metastatic status and pain perception. Clin Biochem 47, 1209-1213.
Papageorgiou, I. E., et al. (2016). TLR4-activated microglia require IFN-gamma to induce severe neuronal dysfunction and death in situ. Proc Natl Acad Sci USA 113, 212-217.
Pevida, M., et al. (2013). Spinal CCL2 and microglial activation are involved in paclitaxel-evoked cold hyperalgesia. Brain Res Bull 95, 21-27.
Prinz, M., Jung, S., and Priller, J. (2019). Microglia Biology: One Century of Evolving Concepts. Cell 179, 292-311.
Rosales, S. L., et al. (2018). A Sensitive and Integrated Approach to Profile Messenger RNA from Samples with Low Cell Numbers. Methods Mol Biol 1799, 275-302.
Seretny, M., et al. (2014). Incidence, prevalence, and predictors of chemotherapy-induced peripheral neuropathy: A systematic review and meta-analysis. Pain 155, 2461-2470.
Singhmar, P., et al., (2020). The fibroblast-derived protein PI16 controls neuropathic pain. Proc Natl Acad Sci USA 117, 5463-5471.
Spann, N.J., et al. (2012). Regulated accumulation of desmosterol integrates macrophage lipid metabolism and inflammatory responses. Cell 151, 138-152.
Stokes, J. A., et al. T. L. (2013). Spinal toll-like receptor signaling and nociceptive processing: regulatory balance between TIRAP and TRIF cascades mediated by TNF and IFNbeta. Pain 154, 733-742.
Tall, A. R., and Yvan-Charvet, L. (2015). Cholesterol, inflammation and innate immunity. Nat Rev Immunol 15, 104-116.
Woller, S. A., et al. (2018). Inhibition of Neuroinflammation by AIBP: Spinal Effects upon Facilitated Pain States. Cell reports 23, 2667-2677.
Woller, S. A., Ravula, S. B., Tucci, F. C., Beaton, G., Corr, M., Isseroff, R. R., Soulika, A. M., Chigbrow, M., Eddinger, K. A., and Yaksh, T. L. (2016). Brain Behav Immun 56, 271-280.
Yan, X., et al., (2019). Interleukin-1beta released by microglia initiates the enhanced glutamatergic activity in the spinal dorsal horn during paclitaxel-associated acute pain syndrome. Glia 67, 482-497.
Yvan-Charvet, L., et al., (2008). Increased inflammatory gene expression in ABC transporter-deficient macrophages: free cholesterol accumulation, increased signaling via toll-like receptors, and neutrophil infiltration of atherosclerotic lesions. Circulation 118, 1837-1847.
Zanoni, I., Tan, Y., Di Gioia, M., Broggi, A., Ruan, J., Shi, J., Donado, C. A., Shao, F., Wu, H., Springstead, J. R., et al. (2016). An endogenous caspase-11 ligand elicits interleukin-1 release from living dendritic cells. Science 352, 1232-1236.
Zhang, M., Li, L., Xie, W., Wu, J. F., Yao, F., Tan, Y. L., Xia, X. D., Liu, X. Y., Liu, D., Lan, G., et al. (2016). Apolipoprotein A-1 binding protein promotes macrophage cholesterol efflux by facilitating apolipoprotein A-1 binding to ABCA1 and preventing ABCA1 degradation. Atherosclerosis 248, 149-159.
Zhang, M., et al. (2018). Apolipoprotein A-1 Binding Protein Inhibits Inflammatory Signaling Pathways by Binding to Apolipoprotein A-1 in THP-1 Macrophages. Circ J 82, 1396-1404.
Zhou, Y., Zhou, B., Pache, L., Chang, M., Khodabakhshi, A. H., Tanaseichuk, O., Benner, C., and Chanda, S. K. (2019). Metascape provides a biologist-oriented resource for the analysis of systems-level datasets. Nat Commun 10, 1523.
Zhu, X., et al., (2010). J Lipid Res 51, 3196-3206.

### REFERENCES FOR EXAMPLE 3

1. Ritter M, et al. Genomics. 2002; 79(5):693-702.
2. Fang L, et al. Control of angiogenesis by AIBP-mediated cholesterol efflux. Nature. 2013; 498(7452): 118-22.
3. Schneider D A, et al. AIBP protects against metabolic abnormalities and atherosclerosis. J Lipid Res. 2018; 59(5):854-63.
4. Choi S H, et al. JCI Insight. 2018; 3(16):pii: 120519.
5. Park S-W, et al. Clinical & Experimental Allergy. 2013; 43(8):914-27.
6. Choi S-H, et al. Arterioscler Thromb Vasc Biol. 2021; 41(2): e82-e96.
7. Das S, et al. Proceedings of the National Academy of Sciences. 2016; 113(46): 13132-7.
8. Akdis C A. Does the epithelial barrier hypothesis explain the increase in allergy, autoimmunity and other chronic conditions? Nat Rev Immunol. 2021; 21(11):739-51.

### REFERENCES FOR EXAMPLE 4

1. John S W, et al. Invest Ophthalmol Vis Sci. 1997; 38:249-53.
2. Libby R T, et al. Vis Neurosci. 2005; 22:637-48.
3. Crish S D, et al. Proc Natl Acad Sci USA. 2010; 107:5196-201.
4. Angelucci A, et al. J Neurosci Methods. 1996; 65:101-12.
5. Cameron E G, et al. Bio Protoc. 2020; 10.

A number of embodiments of the invention have been described. Nevertheless, it will be understood that various modifications may be made without departing from the spirit and scope of the invention. Accordingly, other embodiments are within the scope of the following claims.

1: An isolated or recombinant polypeptide, wherein the polypeptide is comprised of a ApoA-I Binding Protein (AIBP) amino acid sequence and an amino acid sequence N-terminal to the AIBP amino acid sequence,
wherein the amino acid sequence N-terminal to the AIBP amino acid sequence is comprised of at least eight amino acids, or the amino acid sequence N-terminal to the AIBP amino acid sequence is 5, 6, 7, 8, 9, 10, 11, 12 13, 14, 15, or 16 or more amino acids in length,
wherein the amino acid sequence N-terminal to the AIBP amino acid sequence is capable of inducing unfolding, exposing or otherwise making accessible the cryptic domain in the AIBP amino acid sequence for binding of the polypeptide to TLR4 under physiological conditions,
with the proviso that the amino acid sequence N-terminal to the AIBP amino acid sequence is not comprised of a His-tag and a proteolytic cleavage site that when acted upon under said conditions results in loss of the His-tag.

2: The isolated or recombinant polypeptide of claim 1, wherein the amino acid sequence N-terminal to the AIBP amino acid sequence is comprised of between 8 and 40 contiguous amino acid residues acid of which between 3 and 12 amino acid residues are independently selected from the group consisting of arginine (R), histidine (H) and lysine (K).

3: The isolated or recombinant polypeptide compound of claim 1, wherein the N-terminus of the amino acid sequence N-terminal to the AIBP amino acid sequence is a secretion signal amino acid sequence.

4: The isolated or recombinant polypeptide compound of claim 3, wherein the secretion signal amino acid sequence is a fibronectin secretion signal domain, an immunoglobulin heavy chain secretion signal domain, an immunoglobulin kappa light chain secretion signal domain, or an interleukin-2 signal peptide secretion signal domain.

5: The isolated or recombinant polypeptide of claim 4, wherein the fibronectin secretion signal domain is MLRGPGPGRLLLLAVLCLGTSVRCTETGKSKR (SEQ ID: NO:24):

6: The isolated or recombinant polypeptide of claim 1, wherein the AIBP sequence is hAIBP (SEQ ID: No. 6) or d24hAIBP (SEQ ID: No. 8).

7: The isolated or recombinant polypeptide of claim 1, wherein the amino acid sequence N-terminal to the AIBP amino acid sequence is comprised of 6 consecutive histidine amino acid residues (HHHHHH; SEQ ID NO:1), N-terminal to the TLR4 binding domain of the AIBP amino acid sequence.

8: The isolated or recombinant polypeptide compound of claim 7, wherein the polypeptide has a thrombin cleavage domain intervening between the N-terminus of the TLR4 binding domain of the ApoA-I Binding Protein sequence, wherein the thrombin cleavage domain has one or more amino acid deletions and/or mutations within this domain so as to render it functionally inoperable.

9: The isolated or recombinant polypeptide compound of claim 1, wherein the amino acid sequence N-terminal to the AIBP amino acid sequence is:
(SEQ ID NO: 2)
MSPIDPMGHHHHHHGRRRASVAAGILVPRGSPGLDGICSR
or
(SEQ ID NO: 19)
MSPIDPMGHHHHHHGRRRASVAAGILVPRGSDGDDGDDDR,
each having an amino acid mutation of its thrombin cleavage domain so as to render it functionally inoperative.

10: The isolated or recombinant polypeptide of claim 1, wherein the amino acid sequence N-terminal to the AIBP amino acid sequence is selected from the group consisting of:
(SEQ ID NO: 26)
TETGKSKR,
(SEQ ID NO: 33)
MDYKDHDGDYKDHDIDYKDDDDKLAAANS,
and
(SEQ ID NO: 7)
MSPIDPMGHHHHHHGRRRASVAAGILVPAASPGLDGICSR[[,]].

11: The isolated or recombinant polypeptide compound of claim 10, wherein the AIBP amino acid sequence is that of a mammalian AIBP amino acid sequence.

12: The isolated or recombinant polypeptide compound of claim 11, wherein the mammalian AIBP amino acid sequence is that of a human AIBP amino acid sequence,
and optionally the human AIBP amino acid sequence is the full-length amino acid sequence of 288 amino acid residues with NCBI Reference Sequence: NP_658985.2,
and optionally the human AIBP amino acid sequence is the human AIBP amino acid sequence with NCBI Reference Sequence: NP 658985.2 having deletion of amino acids 1-24 from said AIBP amino acid sequence.

13-14. (canceled)

15: A pharmaceutical composition comprised of a polypeptide compound of claim 1 and at least one excipient suitable for parenteral administration.

16: The pharmaceutical composition of claim 15, wherein the pharmaceutical composition is formulated for parenteral administration by intrathecal injection or intrathecal implant.

17: A nucleic acid compound, wherein the nucleic acid compound is comprised of a nucleic acid sequence that encodes for the polypeptide compound of claim 1.

18: An expression vector comprised of a nucleic acid sequence that encodes for the polypeptide compound of claim 1,
wherein optionally the expression vector is a recombinant adenovirus.

19. (canceled)

20: A method for treating, ameliorating, preventing, reversing or decreasing the severity or duration of, or decreasing the severity of symptoms of:
neuropathic pain,
inflammation-induced neuropathic pain,
wherein optionally the inflammation-induced neuropathic pain comprises a Toll-like receptor 4 (TLR4)-mediated inflammation-induced neuropathic pain,
nerve or CNS inflammation,
wherein optionally the nerve or CNS inflammation comprises a TLR4-mediated nerve or CNS inflammation,
allodynia,
wherein optionally the allodynia comprises a TLR4-mediated allodynia,
a post nerve or tissue injury pain or neuropathic pain,
wherein optionally the post nerve or tissue injury pain or neuropathic pain is generated or caused by, or is a sequelae to, trauma, chemotherapy, arthritis, diabetes, or viral infection,
post-surgical pain or neuropathic pain,
chemotherapeutic-induced peripheral neuropathy (CIPN) (for example, a cisplatin-induced CIPN or allodynia),
a neurodegenerative disease or condition, optionally a chronic or progressive neurodegenerative disease or condition, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS),
a primary headache, optionally a migraine or a cluster headache,
hyperalgesia,
glaucoma or other inflammatory diseases of the eye,
lung inflammation and asthma,
acute respiratory distress syndrome (ARDS),
sepsis,
viral infection, and optionally the virus comprises an influenza or a coronavirus (optionally the coronavirus is COVID-19) or a human immunodeficiency virus (HIV) or a virus causing an HIV infection, (optionally an influenza A, B or C), or a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection, or its comorbidities, and/or
vascular inflammation, atherosclerosis and cardiovascular disease,
in a subject by adding or increasing levels of an ApoA-I Binding Protein (APOA1BP, AIBP, or AI-BP),
wherein the method comprises:
(a) providing a formulation or a pharmaceutical composition comprising:
(i) a recombinant or synthetic ApoA-I Binding Protein (APOA1BP, AIBP, or AI-BP) polypeptide compound or composition having a heterologous amino terminus amino acid sequence of at least about ten amino acids, or between about 5 to 20 amino acids, or between about 10 to 100 amino acids, or between about 20 to 80 amino acids, or between about 30 to 50 amino acids, or having on the AIBP amino terminus 5, 6, 7, 8, 9, 10, 11, 12, 13, 14, 15, 16, 17, 18, 19, 20, 21, 22, 23, 24, 25, 26, 27, 28, 29 or 30 or more amino acid residues that are not present in wt AIBP or are non-native (to AIBP) amino acid residues or peptides (also called AIBP variants as provided herein),
and optionally the heterologous amino terminus amino acid sequence comprises a peptide tag, and optionally the peptide tag comprise a multi-histidine (multi-his) tag, and optionally the multi-his tag comprises six histidines (HHHHHH (SEQ ID NO:1)), or 3, 4, 5, 6, 7, 8, 9, 10, 11, 12, 13, 14, 15, 16, 17, 18, 19, or 20 or more histidine residues,
and optionally the heterologous amino terminus amino acid sequence comprises an enzyme cleavage site, and optionally the enzyme cleavage site comprises a thrombin cleavage site,
and optionally the heterologous amino terminus amino acid sequence comprises a secretion signal, and optionally the secretion signal comprises a fibronectin secretion signal, an immunoglobulin heavy chain secretion signal or an immunoglobulin kappa light chain secretory peptide, or an interleukin-2 signal peptide,
and optionally the heterologous amino terminus amino acid sequence comprises the amino acid sequence (SEQ ID NO:2) MSPIDPMGHHHHHHGRRRASVAAGILVPRGSPGLDGICSR,
wherein the variant can unfold or expose or make accessible a cryptic domain in the AIBP molecule, comprising of amino acids 25-51, which mediates AIBP binding to TLR4;
(ii) a recombinant nucleic acid encoding the APOA1BP polypeptide of (i),
and optionally the nucleic acid that expresses or encodes a APOA1BP polypeptide or a polypeptide having a APOA1BP polypeptide activity is contained in an expression vehicle, vector, recombinant virus, or equivalent,
and optionally the vector or virus is or comprises an adenovirus vector or an adeno-associated virus (AAV) vector, a retrovirus, a lentiviral vector, a herpes simplex virus, a human immunodeficiency virus (HIV), or a synthetic vector,
and optionally the AAV vector comprises or is:
an adeno-associated virus (AAV), or an adenovirus vector,
an AAV serotype or variant AAV5, AAV6, AAV8 or AAV9, AAV-DJ or AAV-DJ/8™ (Cell Biolabs, Inc., San Diego, CA),
a rhesus-derived AAV, or the rhesus-derived AAV AAVrh.10hCLN2,
an AAV capsid mutant or AAV hybrid serotype,
an organ-tropic AAV, or a cardiotropic AAV, or a cardiotropic AAVM41 mutant,
wherein optionally the AAV is engineered to increase efficiency in targeting a specific cell type that is non-permissive to a wild type (wt) AAV and/or to improve efficacy in infecting only a cell type of interest,
and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;
(iii) the formulation or pharmaceutical composition of any of (i) to (ii), wherein the recombinant or synthetic ApoA-I Binding Protein (APOA1BP, AIBP, or AI-BP) polypeptide or protein is or comprises all or part of a a human or a mammalian APOA1BP, or a AIBP1 or a AIBP2 sequence;
(iv) a formulation or pharmaceutical composition of any of (i) to (iii), formulated for administration in vivo; or formulated for enteral or parenteral administration, or for oral, intravenous (IV) or intrathecal (IT) administration,
wherein optionally the formulation or pharmaceutical composition, or the recombinant, peptidomimetic or a synthetic APOA1BP, or bioisostere of APOA1BP, or nucleic acid encoding the APOA1BP, or vector having contained therein a nucleic acid encoding the APOA1BP, is carried in a nanoparticle, a particle, a micelle or a liposome or lipoplex, a polymersome, a polyplex or a dendrimer, which optionally can further comprise or express a cell or CNS penetrating moiety or peptide or a CNS targeting moiety or peptide; or
(v) the formulation or pharmaceutical composition of any of (i) to (iv), formulated for as a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant (for example, an intrathecal implant); and
(b) administering the formulation or the pharmaceutical composition of (a) to a subject in need thereof, wherein optionally the subject is a human or an animal, thereby treating, ameliorating, preventing, reversing or decreasing the severity or duration of, or decreasing the severity of symptoms of, the:
neuropathic pain,
inflammation-induced neuropathic pain,
wherein optionally the inflammation-induced neuropathic pain comprises a Toll-like receptor 4 (TLR4)-mediated inflammation-induced neuropathic pain,
nerve or CNS inflammation,
wherein optionally the nerve or CNS inflammation comprises a TLR4-mediated nerve or CNS inflammation,
allodynia,
wherein optionally the allodynia comprises a TLR4-mediated allodynia,
a post nerve or tissue injury pain or neuropathic pain,
wherein optionally the post nerve or tissue injury pain or neuropathic pain is generated or caused by, or is a sequelae to, trauma, chemotherapy, arthritis, diabetes, or viral infection,
post-surgical pain or neuropathic pain,
chemotherapeutic-induced peripheral neuropathy (CIPN) (for example, optionally a cisplatin-induced CIPN or allodynia),
a neurodegenerative disease or condition, optionally a chronic or progressive neurodegenerative disease or condition, optionally Alzheimer's disease or a Chronic Traumatic Encephalopathy (CTE) or a related tauopathy, a traumatic brain injury (TBI), a posttraumatic stress disorder, a traumatic war neurosis, or a post-traumatic stress syndrome (PTSS),
a primary headache, optionally a migraine or a cluster headache,
hyperalgesia,
glaucoma or other inflammatory diseases of the eye,
lung inflammation and asthma,
acute respiratory distress syndrome (ARDS),
sepsis,
viral infection, and optionally the virus comprises an influenza or a coronavirus (optionally the coronavirus is COVID-19) or a human immunodeficiency virus (HIV) or a virus causing an HIV infection, (optionally an influenza A, B or C), or a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection, or its comorbidities, and/or.
vascular inflammation, atherosclerosis and cardiovascular disease.

21: A kit comprising a recombinant or isolated polypeptide of claim 1.

22-24. (canceled)

25: A method for exposing the cryptic N-terminal TLR4-binding domain of an ApoA-I Binding Protein (APOA1BP, AIBP, or AI-BP) polypeptide, comprising adding to a native AIBP polypeptide a heterologous amino terminus amino acid sequence of at least about ten amino acid, or between about 5 to 50 amino acids, or between about 10 to 100 amino acids, or adding about 20 to 80 amino acids, or between about 30 to 50 amino acids, or adding on the amino terminus 5, 6, 7, 8, 9, 10, 11, 12, 13, 14, 15, 16, 17, 18, 19, 20, 21, 22, 23, 24, 25, 26, 27, 28, 29 or 30 or more amino acid residues that are not present in wt AIBP or are non-native (non-AIBP) amino acid residues or peptides,
and optionally the heterologous amino terminus amino acid sequence comprises a peptide tag, and optionally the peptide tag comprises a multi-histidine (multi-his) tag, and optionally the multi-his tag comprises at least six histidines (HHHHHH (SEQ ID NO:1)), or 3, 4, 5, 6, 7, 8, 9, 10, 11, 12, 13, 14, 15, 16, 17, 18, 19, or 20 or more histidine residues,
and optionally the heterologous amino terminus amino acid sequence comprises an enzyme cleavage site, and optionally the enzyme cleavage site comprises a thrombin cleavage site,
and optionally the heterologous amino terminus amino acid sequence comprises a secretion signal, and optionally the secretion signal comprises a fibronectin secretion signal, an immunoglobulin heavy chain secretion signal or an immunoglobulin kappa light chain secretory peptide, or an interleukin-2 signal peptide,
and optionally the heterologous amino terminus amino acid sequence comprises the amino acid sequence (SEQ ID NO:2)
MSPIDPMGHHHHHHGRRRASVAAGILVPRGSPGLDGICSR.

26: The method of claim 1, wherein the physiological conditions comprise conditions experienced by the polypeptide compound in vivo upon providing it to a subject in need thereof by administration,
